(19) United States
(12) Patent Application Publication
(10) Pub. No.: US 2024/0374590 A1
(43) Pub. Date: Nov. 14, 2024

(54) COMPOSITIONS AND METHODS FOR TREATING CANCER

(71) Applicants: A2A PHARMACEUTICALS, INC., New York, NY (US); ONCOCUBE THERAPEUTICS LLC, Wilmington, DE (US)

(72) Inventors: Chaemin Lim, New York, NY (US); Erden Banoglu, Ankara (TR); Burcu Caliskan, Ankara (TR); Ozgur Sahin, Lexington, SC (US); Sridhar Vempati, Edison, NJ (US); Deniz Lengerli, Ankara (TR); Kubra Ibis, Ankara (TR); Emmanuel Cruz, West Long Branch, NJ (US)

(21) Appl. No.: 18/286,469

(22) PCT Filed: Apr. 11, 2022
(86) PCT No.: PCT/US22/24263

§ 371 Date: Oct. 11, 2023

Related U.S. Application Data
(60) Provisional application No. 63/173,796, filed on Apr. 12, 2021.

(51) Int. Cl. A61K 31/506; A61K 31/4439; A61K 31/497; A61K 31/53; A61K 31/553; A61P 35/00; C07D 401/12; C07D 401/14; C07D 405/14; C07D 413/14

(52) U.S. Cl. CPC A61K 31/506; A61K 31/4439; A61K 31/497; A61K 31/53; A61K 31/553; A61P 35/00; C07D 401/12; C07D 401/14; C07D 405/14; C07D 413/14

(57) ABSTRACT

Disclosed herein are inhibitors of TACC and methods of treating certain diseases and disorders (e.g., diseases and disorders related to TACC).

### RELATED APPLICATIONS

The instant application claims priority to U.S. Provisional Patent Application No. 63/173,796, filed Apr. 12, 2021, the contents of which are hereby fully incorporated by reference.

### BACKGROUND

Cancer is a complex disease characterized by uncontrolled cell division. In the USA, among cancer types, breast cancer, lung cancer and colorectal cancer account for 50% of all cases in women while prostate, lung, and colorectal cancers account for 46% of all newly diagnosed cases in men (Siegel et al., 2021). Although, the Food and Drug Administration (FDA) grants approval to several novel drugs and new indications for therapeutic agents currently in clinical use for cancer treatment, there are still millions of cancer deaths worldwide each year.

With the understanding of tumor biology, targeted medical therapies have continuously been developed to increase the patient survival rate. In view of the side effects of currently available chemotherapy agents, the development of targeted therapies causing less toxicity has been a major focus in recent years. Since cancer is characterized as abnormal and uncontrollable cell growth with the potential to invade or spread to the other parts of the body or a malignant tumor, drugs or substances that target and inhibit the function of specific macromolecules responsible for the proliferation and survival of tumor cells are used in cancer-targeted therapies.

Microtubule re-organization is an important step during cell division and drugs that interfere with this process have been a major focus of cancer research. Anti-mitotic drugs disrupt the polymerization dynamics of microtubules by activating the spindle assembly check point (SAC), which prevents the transition from metaphase to anaphase. As a result, cells stop division, and these mitotically arrested cells die. A continuous investigation of the mechanism of mitotic events may lead to new target protein candidates and/or pathways for the treatment of cancer. Anti-microtubule agents, such as vinca alkaloids, maytansinoids and taxanes are examples of such drugs that are widely used as chemotherapeutic agents for a variety of tumors (Marzo & Naval, 2013). However, these compounds are toxic to non-tumorigenic cells and can result in serious side effects. Drug resistance is also another major problem causing patients' response to these drugs highly unpredictable (Gascoigne & Taylor, 2009). To overcome these problems and improve chemotherapy response, anti-mitotic cancer specific therapies targeting mitosis-specific kinases and microtubule-motor proteins have been identified (Dominguez-brauer et al., 2015). Importantly, since phosphorylation is a critical step in cell cycle regulation and spindle assembly, kinases having role in these processes have been identified as potential targets. Among these, specific inhibitors against cyclin-dependent kinases (Cdks), aurora kinases and polo-like kinases (PLKs) have been developed and clinically tested (Sanchez-martinez, Gelbert, Lallena, & De dios, 2015; Strebhardt & Ullrich, 2006; Tang et al., 2017). Compared to anti-microtubule agents, and despite their reduced toxicity, none of these anti-mitotic drugs have good clinical outcomes (Chan, Koh, & Li, 2012). The lack of efficacy of anti-mitotic drugs may be attributed, in part, to the fact that cancer cells in clinical tumors are predominantly at the interphase (Ogden et al., 2013). Therefore, therapeutic strategies that target not only mitosis but also the interphase-specific activities, such as migration or transcriptional reprogramming, as well as those that evoke anti-tumor immunity may provide superior effects in tumors resistant to microtubule targeting agents. Such therapies are expected to have high translational potential that will ultimately improve clinical outcome.

### SUMMARY OF THE INVENTION

In one aspect, the present disclosure provides compounds of formula I or a pharmaceutically acceptable salt thereof:

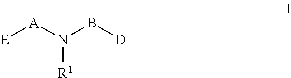

wherein,
E and B are each independently aryl, heteroaryl, or heterocyclyl;
D is heterocyclyl;
A is heteroaryl; and
R¹ is H, alkyl, or benzyl.

In another aspect, the present disclosure provides a pharmaceutical composition comprising a compound disclosed herein and a pharmaceutically acceptable excipient.

In yet another aspect, the present disclosure provides methods of treating TACC mediated diseases or disorders in a subject comprising administering a compound disclosed herein or a pharmaceutically acceptable salt thereof to the subject.

### BRIEF DESCRIPTION OF THE DRAWINGS

FIG. 1 shows in vivo activity of an exemplary compound of the disclosure. Tumor growth inhibition was observed at certain dose levels.

### DETAILED DESCRIPTION OF THE INVENTION

Transforming acidic coiled-coil proteins (TACC) family members are emerging as important proteins for microtubule and centrosome related functions. Vertebrates express 3 different isoforms of TACC: TACC1, TACC2, and TACC3. TACC has been found to play a critical role in gene regulation, cell growth and differentiation, mRNA processing, transcription, migration and so on by interacting with different molecules involved in microtubule/centrosome dynamics (Ha et al., 2013). Members share a conserved domain, called TACC domain, which is required for TACC proteins to interact with spindles and centrosome apparatus (Gergely et al., 2000). Although the members of TACC family were described as centrosomal proteins, they are also distributed throughout the cell during interphase. For instance, TACC3 and TACC2 form a complex with different histone acetyltransferases, including hGCN5L2 and pCAF showing their regulatory function in transcription (Gangisetty et al., 2004). Noticeably, TACC3 interacts with MBD2 (mCpG-binding domain 2) in the interphase nucleus where it facilitates the association of MBD2 with histone acetyltransferases to reactivate methylated promoters.

TACC proteins levels are elevated in many cancer types including prostate cancer, hepatocellular carcinoma, non-small cell lung cancer and breast cancer and so on. TACC1, first member of TACC family, was independently discovered as a breast cancer amplicon 8p11 (Still et al., 1999) and later found to be able to promote mammary tumorigenesis possibly through the activation of Ras/PI3K signaling pathways (Cully et al., 2005). TACC2 has been found to promote androgen mediated growth in the prostate cancer and is associated with poor prognosis (Takayama et al., 2012). Furthermore, the overexpression of TACC2 leads to proliferation of breast cancer cells (Cheng et al., 2010). TACC3, when disrupted, also causes a range of different cellular outcomes including multi-polar spindle formation leading to mitotic arrest (Yao et al., 2012), chromosome misalignment resulting in caspase-dependent apoptosis (Schneider et al., 2007) and, in some cases, senescence (Schmidt et al., 2010). Moreover, knockdown of TACC3 suppresses tumorigenesis and cell growth in renal cell carcinoma (RCC) (Guo & Liu, 2018). The aforementioned studies show that the TACC family of proteins are critical molecules enrolled in spindle assembly of cancer cells, which makes them important and potential targets for cancer targeted therapy.

However, to date, there is no available inhibitor for TACC1 and TACC2 and there are merely two inhibitors targeting TACC3. KHS101, a small molecule TACC3 inhibitor, was first identified to promote neuronal differentiation in rats (Wurdak et al., 2010). Although tumor growth of glioblastoma (GBM) xenografts were suppressed through KHS101 treatment (Polson et al., 2018), KHS101 has many drawbacks, such low oral systemic stability and high working doses (Wurdak et al., 2010). Another TACC3 inhibitor, SPL-B, has been shown to inhibit the centrosome microtubule nucleation in ovarian cancer cells and suppress tumor growth in ovarian cancer xenografts (Yao et al., 2014). However, like KHS101, SPL-B has not been approved for the treatment of cancer.

In view of the foregoing, there is a clear, unmet need, for new TACC inhibitors for the treatment of cancer and other TACC mediated diseases.

In one aspect, the present disclosure provides compounds of formula I or a pharmaceutically acceptable salt thereof:

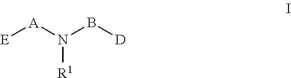

wherein,
E and B are each independently aryl, heteroaryl, or heterocyclyl;
D is heterocyclyl;
A is heteroaryl; and
R¹ is H, alkyl, or benzyl.

In certain preferred embodiments, A is not isoxazole.

In certain embodiments, A is pyrrole, furan, selenophene, thiophene, imidazole, pyrazole, oxazole, oxathiole, isoxathiole, thiazole, isothiazole, triazole, furazan, oxadiazole, thiadiazole, dioxazole, or dithiazole. In certain preferred embodiments, A is pyrazole.

In certain embodiments, the compound is represented by formula Ia or Ib:

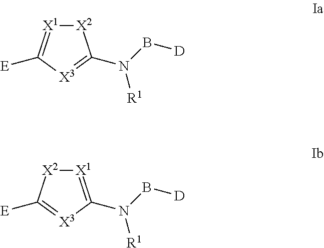

wherein,
E and B are each independently aryl, heteroaryl, or heterocyclyl;
D is heterocyclyl;
X¹ is selected from CH₂, NR², O, and S;
X² is selected from CH or N;
X³ is CR³ or N;
R¹ and R² are each independently H, alkyl, or benzyl; and
R³ is H, alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, or sulfonamide.

In certain embodiments, the compound is represented by formula Ta:

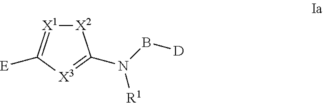

wherein,
E and B are each independently aryl, heteroaryl, or heterocyclyl;
D is heterocyclyl;
X¹ is C or N;
X² is CH₂, NR², O, or S;
X³ is CR³ or N;
R¹ and R² are each independently H, alkyl, or benzyl; and
R³ is alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, or sulfonamide.

In certain embodiments, X¹ is N. In other embodiments, X¹ is CH.

In certain embodiments, X² is NR². In certain embodiments, R² is H. In other embodiments, R² is alkyl. In certain preferred embodiments, R² is methyl.

In certain embodiments, X² is S. In other embodiments, X² is O.

In certain embodiments, X³ is CR³. In certain embodiments, R³ is H. In other embodiments, R³ is alkyl. In certain embodiments, R³ is methyl.

In certain embodiments, R¹ is H. In other embodiments, R¹ is alkyl. In certain embodiments, R¹ is methyl or ethyl.

In certain embodiments, B is heteroaryl. In certain embodiments, B is pyridinyl, pyrimidinyl, or triazinyl. In certain preferred embodiments, B is pyrimidinyl.

In certain embodiments, B is substituted with at least one R⁴ and each R⁴ is independently selected from alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide. In certain embodiments, B is substituted with at least one R⁴ and each R⁴ is independently selected from alkyl and halo. In certain preferred embodiments, R⁴ is methyl. In other preferred embodiments, R⁴ is chloro or fluoro. In certain embodiments, B is substituted with 1 or 2 R⁴.

In certain embodiments, D is N-linked heterocyclyl, such as a monocyclic or bicyclic heterocyclyl. In certain embodiments, D is aziridinyl, azetidinyl, pyrrolidinyl, piperidinyl, azepanyl, piperazinyl, pyranyl, dihydropyranyl, morpholinyl, thiomorpholinyl, dioxidethiomorpholinyl, oxaazabicycloheptanyl, azabicyclooctanyl, oxaazabicyclooctanyl, hexahydrofuropyrrolyl, or azabicyclohexanyl. In certain embodiments, D is azetidinyl, pyrrolidinyl, piperidinyl, piperazinyl, morpholinyl, thiomorpholinyl, dioxidethiomorpholinyl, azabicyclooctanyl, oxaazabicyclooctane, hexahydrofuropyrrolyl, or azabicyclohexanyl. In certain embodiments, D is oxaazabicycloheptanyl, azabicyclooctanyl, or oxaazabicyclooctanyl.

In certain embodiments, D is substituted with at least one R⁵ and each R⁵ is independently selected from H, deuterium, alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide. In certain embodiments, D is substituted with at least two R⁵s and two of the R⁵s combine to complete a bicyclic heterocyclyl. In certain embodiments, D is substituted with at least one R⁵ and each R⁵ is independently selected from alkyl, halo, cycloalkyl, or heterocyclyl. In certain embodiments, R⁵ is methyl, fluoromethyl, difluoromethyl, trifluoromethyl, fluoor, cyclopropyl, cyclobutyl, or oxetanyl. In certain embodiments, D is substituted with 1 or 2 R⁵. In certain embodiments, D is substituted with 1 R⁵. In other embodiments, D is substituted with 2 R⁵s.

In certain embodiments, E is aryl. In certain embodiments, E is phenyl, dihydrobenzofuranyl, benzodioxolyl, or dihydroindenyl. In certain embodiments, E is phenyl, dihydrobenzofuran, or benzodioxole. In certain preferred embodiments, E is phenyl. In other embodiments, E is heteroaryl. In certain embodiments, E is pyridinyl, pyrazinyl, indolyl, such as N-methyl indolyl, or benzofuranyl. In certain embodiments, E is pyridinyl or pyrazinyl. In yet other embodiments, E is heterocyclyl. In certain embodiments, E is pyrrolidinyl.

In certain embodiments, E is substituted with at least one R⁶ and each R⁶ is independently selected from alkyl with alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfomide.

In certain embodiments, E is substituted with at least one R⁶ and each R⁶ is independently selected from alkyl (e.g., deuteroalkyl), alkyloxy (e.g., deuteroalkyloxy), alkylthio, amino, halo, cyano, heterocyclyl, and hydroxyl. In certain embodiments, R⁶ is methyl, ethyl, butyl, isopropyl, difluoromethyl, trifluoromethyl, difluoroethyl, methoxy, ethoxy, difluoromethyoxy, trifluoromethyoxy, methylthio, dimethylamino, fluoro, chloro, or azetidinyl. In certain embodiment, E is substituted with 1 R⁶. In other embodiments, E is substituted with 2 R⁶. In yet other embodiments, E is substituted with 3 R⁶.

In certain embodiments, the compound is represented by formula Ic or a pharmaceutically

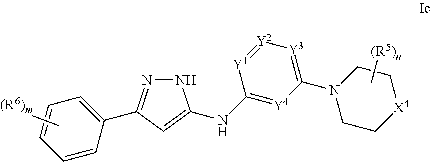

wherein,
Y¹ is N or CR^{8a};
Y² is N or CR^{8b};
Y³ is N or CR^{8c}.
Y⁴ is N or CR^{8d};
X⁴ is CR^{5c}R^{5d}, O, or NR⁷;
R^{5c} and R^{5d} are each independently selected from deuterium, alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide; or R^{5c} and R^{5d} combine to form cycloalkyl;
R^{8a}, R^{8b}, R^{8c}, and R^{8d} are each independently selected from H, deuterium, alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide;
R⁷ is H, alkyl, acyl, acetyl, hydroxyl, alkoxy, cycloalkyl;
m is 1-5; and
n is 1-8.

In certain embodiments, Y¹ is N. In other embodiments, Y¹ is CR^{8a}. In certain embodiments, R^{8a} is H, alkyl (e.g., methyl) or halo (e.g., fluoro or chloro). In certain preferred embodiments, R^{8a} is fluoro.

In certain embodiments, Y² is N. In other embodiments, Y² is CR^{8b}. In certain embodiments, R^{8b} is H, alkyl (e.g., methyl) or halo (e.g., fluoro or chloro). In certain preferred embodiments, R^{8b} is fluoro.

In certain embodiments, Y³ is N. In other embodiments, Y³ is CR^{8c}. In certain embodiments, R^{8c} is H, alkyl (e.g., methyl) or halo (e.g., fluoro or chloro). In certain preferred embodiments, R^{8c} is fluoro.

In certain embodiments, Y⁴ is N. In other embodiments, Y⁴ is CR^{8d}. In certain embodiments, R^{8d} is H, alkyl (e.g., methyl) or halo (e.g., fluoro or chloro). In certain preferred embodiments, R^{8d} is fluoro.

In certain embodiments, the compound is represented by formula II or a pharmaceutically acceptable salt thereof:

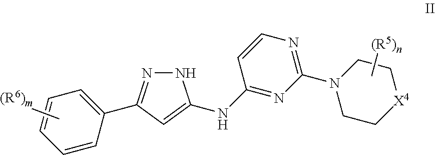

wherein,
X⁴ is CR^{5c}R^{5d}, O, or NR⁷;
R^{5c} and R^{5d} are each independently selected from deuterium, alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide; or R^{5′} and R^{5d} combine to form cycloalkyl;
R⁷ is H, alkyl, acyl, acetyl, hydroxyl, alkoxy, cycloalkyl;
m is 1-5; and
n is 1-8.

In certain embodiments, X⁴ is NR⁷. In certain preferred embodiments, X⁴ is O. In other preferred embodiments, X⁴ is CR^{5c}R^{5d}.

In certain embodiments, the compound is represented by formula IIIa or a pharmaceutically acceptable salt thereof:

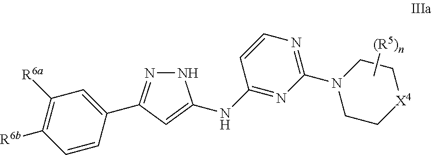

wherein,
R^{6a} and R^{6b} are each independently selected from H, deuterium, alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide.

In certain embodiments, the compound is represented by formula IIIb or a pharmaceutically acceptable salt thereof:

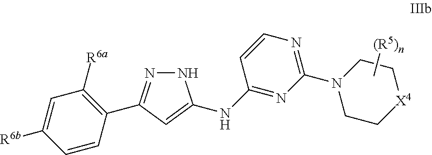

wherein,
R^{6a} and R^{6b} are each independently selected from H, deuterium, alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide.

In certain embodiments, n is at least 2 and two or more R⁵s combine to form a cycloalkyl or heterocyclyl (e.g., a oxaazabicycloheptanyl, azabicyclooctanyl, or oxaazabicyclooctanyl).

In certain embodiments, the compound is represented by formula IVa or a pharmaceutically acceptable salt thereof:

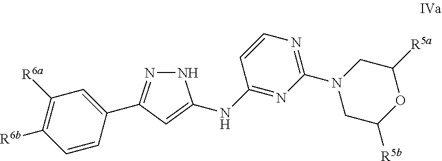

wherein,
R^{5a}, R^{5b}, R^{6a}, and R^{6b} are each independently selected from H, deuterium, alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide.

In certain embodiments, the compound is represented by formula IVb or a pharmaceutically acceptable salt thereof:

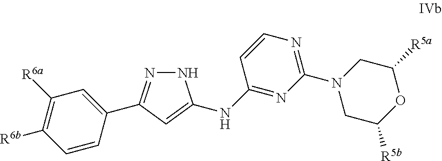

wherein,
R^{5a}, R^{5b}, R^{6a}, and R^{6b} are each independently selected from H, deuterium, alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide.

In certain embodiments, the compound is represented by formula IVc or a pharmaceutically acceptable salt thereof:

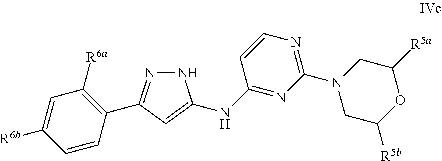

wherein,
R^{5a}, R^{5b}, R^{6a}, and R^{6b} are each independently selected from hydrogen, alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide.

In certain embodiments, the compound is represented by formula IVd or a pharmaceutically acceptable salt thereof:

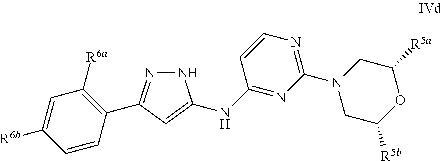

wherein,
R^{5a}, R^{5b}, R^{6a}, and R^{6b} are each independently selected from hydrogen, alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide.

In certain embodiments, the compound is represented by formula IVe or a pharmaceutically acceptable salt thereof:

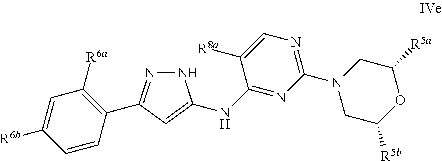

wherein,
R^{5a}, R^{5b}, R^{6a}, and R^{6b} are each independently selected from hydrogen, alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide; and
R^{8a} is selected from hydrogen, deuterium, alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide.

In certain embodiments, R^{8a} is halo (e.g., fluoro or chloro). In certain preferred embodiments, R^{8a} is fluoro. In other embodiments, R^{8a} is alkyl (e.g., methyl).

In certain embodiments, the compound is represented by formula IVf or a pharmaceutically acceptable salt thereof:

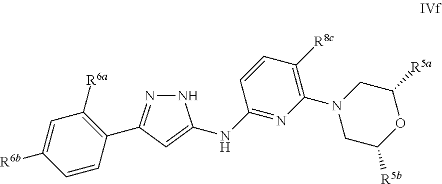

wherein,
R^{5a}, R^{5b}, R^{6a}, and R^{6b} are each independently selected from hydrogen, alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide; and
R^{8c} is selected from hydrogen, deuterium, alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide.

In certain embodiments, R^{8c} is halo (e.g., fluoro or chloro). In certain preferred embodiments, R^{8c} is fluoro. In other embodiments, R^{8c} is alkyl (e.g., methyl).

In certain embodiments, the compound is represented by formula IVg or a pharmaceutically acceptable salt thereof:

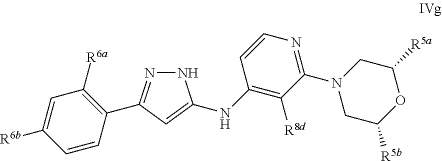

wherein,
R^{5a}, R^{5b}, R^{6a}, and R^{6b} are each independently selected from hydrogen, alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide; and
R^{8d} is selected from hydrogen, deuterium, alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide.

In certain embodiments, R^{8d} is halo (e.g., fluoro or chloro). In certain preferred embodiments, R^{8d} is fluoro. In other embodiments, R^{8d} is alkyl (e.g., methyl).

In certain embodiments, the compound is represented by formula IVh or a pharmaceutically acceptable salt thereof:

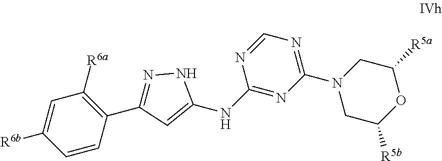

wherein,
R^{5a}, R^{5b}, R^{6a}, and R^{6b} are each independently selected from hydrogen, alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide;

In certain embodiments, R^{5a} is alkyl. In certain preferred embodiments, R^{5a} is methyl.

In certain embodiments, R^{5b} is alkyl. In certain preferred embodiments, R^{5b} is methyl.

In certain embodiments, the compound is represented by formula Va or a pharmaceutically acceptable salt thereof:

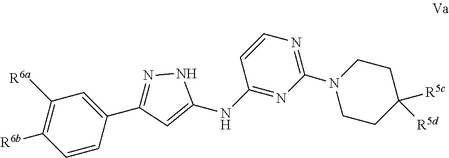

wherein,
R^{8c} and R^{5d} are each independently selected from alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide; or R^{5c} and R^{5d} combine to form cycloalkyl; and
R^{6a} and R^{6b} are each independently selected from alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide.

In certain embodiments, the compound is represented by formula Vb or a pharmaceutically acceptable salt thereof:

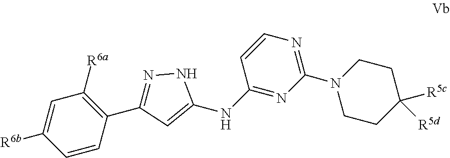

wherein,
R^{5c} and R^{5d} are each independently selected from alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide; or R^{5c} and R^{5d} combine to form cycloalkyl; and
R^{6a} and R^{6b} are each independently selected from alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide.

In certain embodiments, the compound is represented by formula VIa or a pharmaceutically acceptable salt thereof:

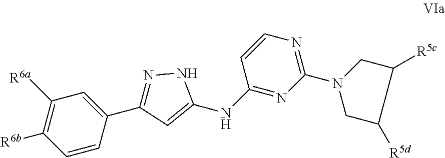

wherein,
R^{5c} and R^{5d} are each independently selected from alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide; or R^{5c} and R^{5d} combine to form cycloalkyl; and
R^{6a} and R^{6b} are each independently selected from alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide.

In certain embodiments, the compound is represented by formula VIb or a pharmaceutically acceptable salt thereof:

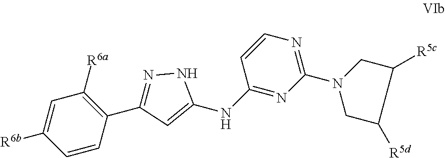

wherein,
R^{5c} and R^{5d} are each independently selected from alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide; or R^{5c} and R^{5d} combine to form cycloalkyl; and
R^{6a} and R^{6b} are each independently selected from alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide.

In certain embodiments, R^{6a} is halo. In certain embodiments R^{6a} is fluoro, chloro, or bromo. In certain preferred embodiments, R^{6a} is fluoro. In other embodiments, R^{6a} is alkyl (e.g., deuteroalkyl).

In certain embodiments, R^{6b} is alkoxy (e.g., deuteroalkyloxy). In certain embodiments, R^{6b} is deuteroalkoxy, methoxy, or fluoromethyoxy, such as monofluoromethyoxy or difluoromethyoxy. In other embodiments, R^{6b} is alkyl (e.g., deuteroalkyl). In certain embodiments, R^{6b} is methyl, ethyl, fluoroalkyl, such as monofluoromethyl, difluoromethyl, or difluoroethyl. In certain preferred embodiments, R^{6b} is fluoroalkyl. In further preferred embodiments, R^{6b} is difluoromethyl. In other embodiments R^{6b} is alkylsulfonyl. In certain embodiments, R^{6b} is methylsulfonyl.

In certain embodiments, R^{5c} is alkyl (e.g., deuteroalkyl). In certain preferred embodiments, R^{5c} is methyl or trifluoromethyl. In other embodiments, R⁵, is halo. In certain preferred embodiments, R^{5c} is fluoro.

In certain embodiments, R^{5d} is hydrogen. In other embodiments, R^{5d} is alkyl (e.g., deuteroalkyl).

In other embodiments, R^{5c} and R^{5d} combine to form cycloalkyl. In certain preferred embodiments, cyclopropyl or cyclobutyl.

In certain embodiments, the compound is selected from a compound recited in Table 1 or a pharmaceutically acceptable salt thereof:

TABLE 1
Exemplary Compounds of the Present Disclosure

| Compound | |
|---|---|
| No. | Molecular Formula |
| 5 | 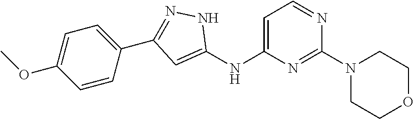 [SMILES: COc1ccc(-c2cc(Nc3ccnc(N4CCOCC4)n3)[nH]n2)cc1] |
| 6 | 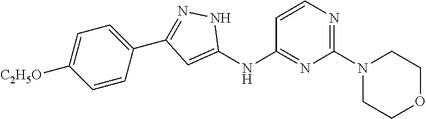 |
| 7 | 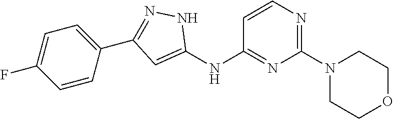 [SMILES: Fc1ccc(-c2cc(Nc3ccnc(N4CCOCC4)n3)[nH]n2)cc1] |
| 8 | 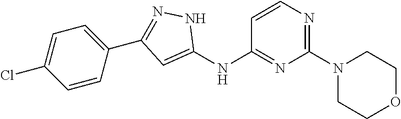 [SMILES: Clc1ccc(-c2cc(Nc3ccnc(N4CCOCC4)n3)[nH]n2)cc1] |
| 9 | 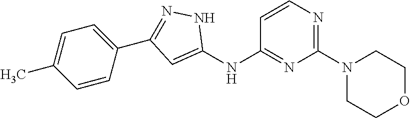 [SMILES: Cc1ccc(-c2cc(Nc3ccnc(N4CCOCC4)n3)[nH]n2)cc1] |
| 10 | 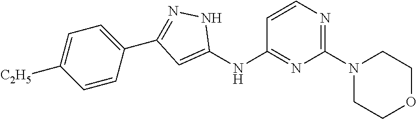 [SMILES: CCc1ccc(-c2cc(Nc3ccnc(N4CCOCC4)n3)[nH]n2)cc1] |
| 11 | 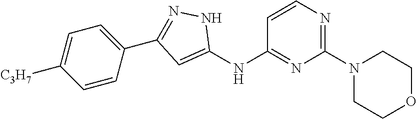 |
| 12 | 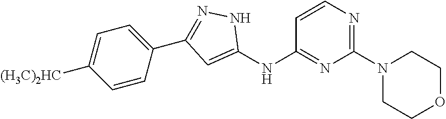 |
| 13 | 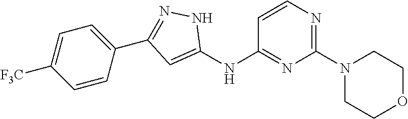 [SMILES: FC(F)(F)c1ccc(-c2cc(Nc3ccnc(N4CCOCC4)n3)[nH]n2)cc1] |
| 14 | 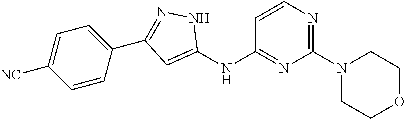 [SMILES: N#Cc1ccc(-c2cc(Nc3ccnc(N4CCOCC4)n3)[nH]n2)cc1] |
| 15 | 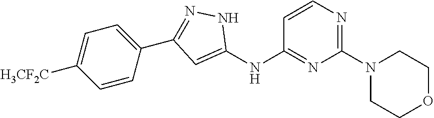 |
| 16 | 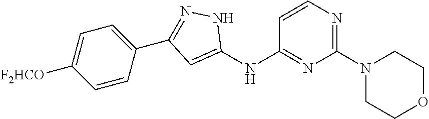 [SMILES: FC(F)Oc1ccc(-c2cc(Nc3ccnc(N4CCOCC4)n3)[nH]n2)cc1] |
| 17 | 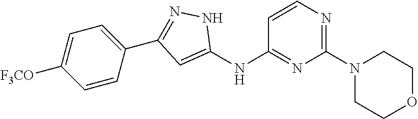 [SMILES: FC(F)(F)Oc1ccc(-c2cc(Nc3ccnc(N4CCOCC4)n3)[nH]n2)cc1] |
| 18 | 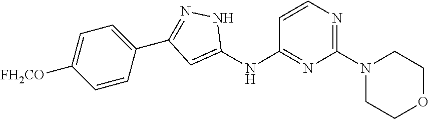 |
| 19 | 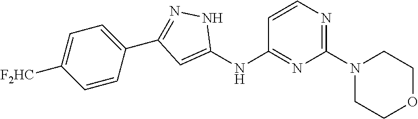 [SMILES: FC(F)c1ccc(-c2cc(Nc3ccnc(N4CCOCC4)n3)[nH]n2)cc1] |
| 20 | 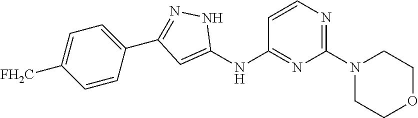 |
| 21 | 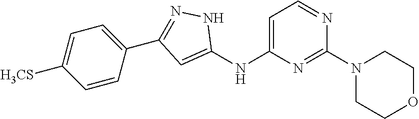 |
| 22 | 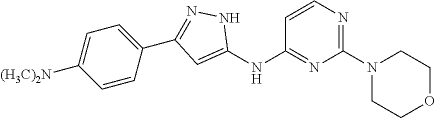 |
| 23 | 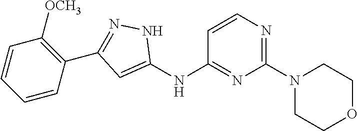 [SMILES: COc1ccccc1-c1cc(Nc2ccnc(N3CCOCC3)n2)[nH]n1] |
| 24 | 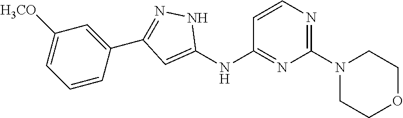 [SMILES: COc1cccc(-c2cc(Nc3ccnc(N4CCOCC4)n3)[nH]n2)c1] |
| 25 | 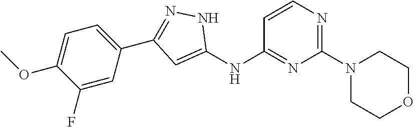 [SMILES: COc1ccc(-c2cc(Nc3ccnc(N4CCOCC4)n3)[nH]n2)cc1F] |
| 26 | 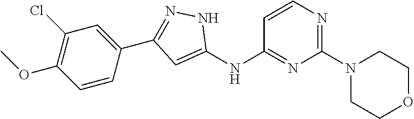 [SMILES: COc1ccc(-c2cc(Nc3ccnc(N4CCOCC4)n3)[nH]n2)cc1Cl] |
| 27 | 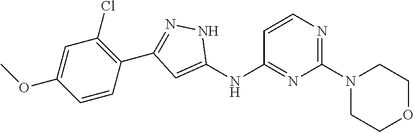 [SMILES: COc1ccc(-c2cc(Nc3ccnc(N4CCOCC4)n3)[nH]n2)c(Cl)c1] |
| 28 | 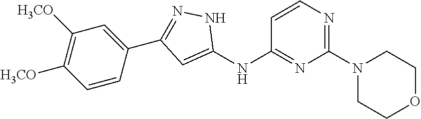 [SMILES: COc1ccc(-c2cc(Nc3ccnc(N4CCOCC4)n3)[nH]n2)cc1OC] |
| 29 | 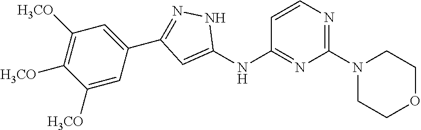 [SMILES: COc1cc(-c2cc(Nc3ccnc(N4CCOCC4)n3)[nH]n2)cc(OC)c1OC] |
| 30 | 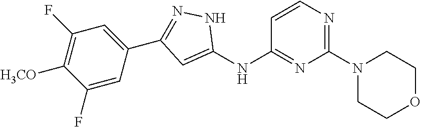 [SMILES: COc1c(F)cc(-c2cc(Nc3ccnc(N4CCOCC4)n3)[nH]n2)cc1F] |
| 31 | 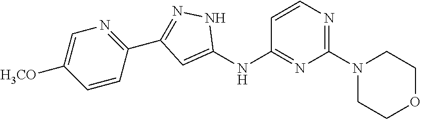 [SMILES: COc1ccc(-c2cc(Nc3ccnc(N4CCOCC4)n3)[nH]n2)nc1] |
| 32 | 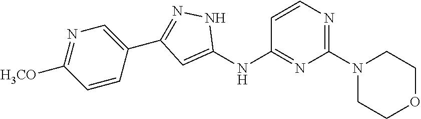 [SMILES: COc1ccc(-c2cc(Nc3ccnc(N4CCOCC4)n3)[nH]n2)cn1] |
| 33 | 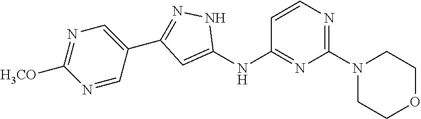 [SMILES: COc1ncc(-c2cc(Nc3ccnc(N4CCOCC4)n3)[nH]n2)cn1] |
| 34 | 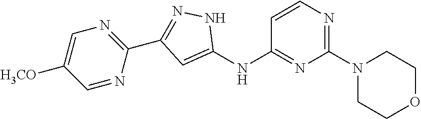 [SMILES: COc1cnc(-c2cc(Nc3ccnc(N4CCOCC4)n3)[nH]n2)nc1] |
| 35 | 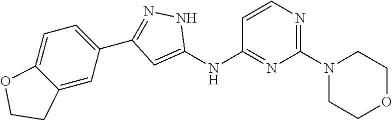 [SMILES: c1cc(Nc2cc(-c3ccc4c(c3)CCO4)n[nH]2)nc(N2CCOCC2)n1] |
| 36 | 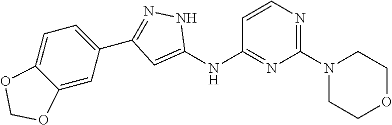 [SMILES: c1cc(Nc2cc(-c3ccc4c(c3)OCO4)n[nH]2)nc(N2CCOCC2)n1] |
| 37 | 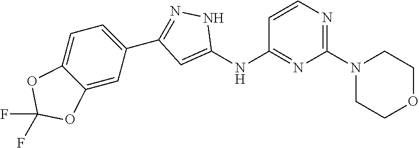 [SMILES: FC1(F)Oc2ccc(-c3cc(Nc4ccnc(N5CCOCC5)n4)[nH]n3)cc2O1] |
| 38 | 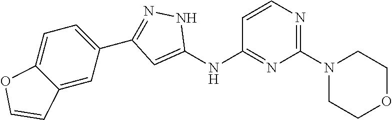 [SMILES: c1cc(Nc2cc(-c3ccc4occc4c3)n[nH]2)nc(N2CCOCC2)n1] |
| 39 | 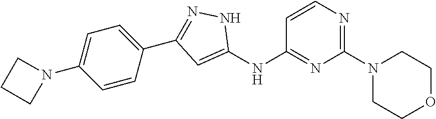 [SMILES: c1cc(Nc2cc(-c3ccc(N4CCC4)cc3)n[nH]2)nc(N2CCOCC2)n1] |
| 40 | 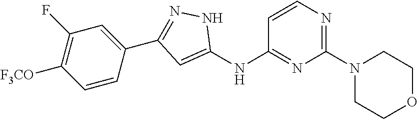 [SMILES: Fc1cc(-c2cc(Nc3ccnc(N4CCOCC4)n3)[nH]n2)ccc1OC(F)(F)F] |
| 41 | 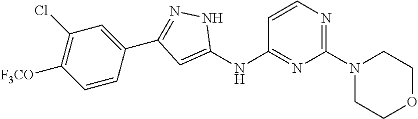 [SMILES: FC(F)(F)Oc1ccc(-c2cc(Nc3ccnc(N4CCOCC4)n3)[nH]n2)cc1Cl] |
| 43 | 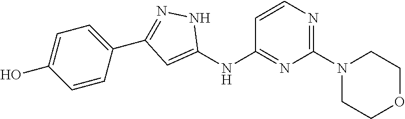 [SMILES: Oc1ccc(-c2cc(Nc3ccnc(N4CCOCC4)n3)[nH]n2)cc1] |
| 56 | 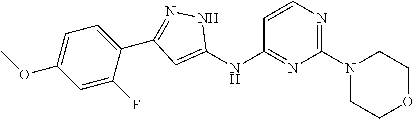 [SMILES: COc1ccc(-c2cc(Nc3ccnc(N4CCOCC4)n3)[nH]n2)c(F)c1] |
| 57 | 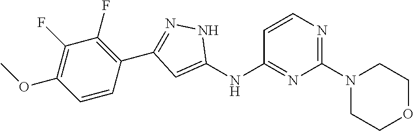 [SMILES: COc1ccc(-c2cc(Nc3ccnc(N4CCOCC4)n3)[nH]n2)c(F)c1F] |
| 58 | 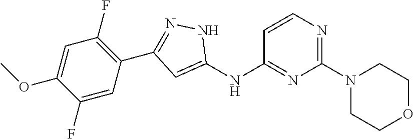 [SMILES: COc1cc(F)c(-c2cc(Nc3ccnc(N4CCOCC4)n3)[nH]n2)cc1F] |
| 59a | 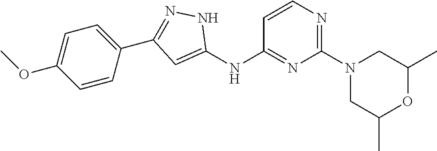 |
| 59b | 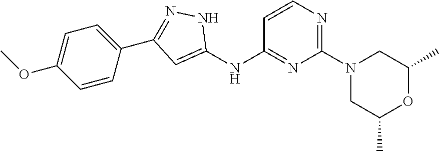 |
| 60 | 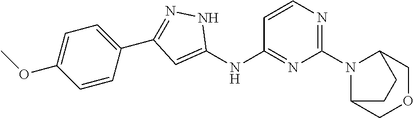 [SMILES: COc1ccc(-c2cc(Nc3ccnc(N4C5CCC4COC5)n3)[nH]n2)cc1] |
| 61 | 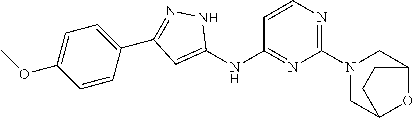 [SMILES: COc1ccc(-c2cc(Nc3ccnc(N4CC5CCC(C4)O5)n3)[nH]n2)cc1] |
| 62 | 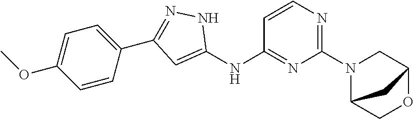 [SMILES: COc1ccc(-c2cc(Nc3ccnc(N4C[C@H]5C[C@@H]4CO5)n3)[nH]n2)cc1] |
| 63 | 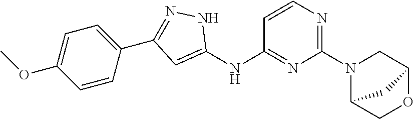 [SMILES: COc1ccc(-c2cc(Nc3ccnc(N4C[C@@H]5C[C@H]4CO5)n3)[nH]n2)cc1] |
| 64 | 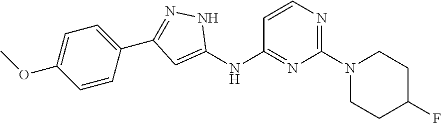 [SMILES: COc1ccc(-c2cc(Nc3ccnc(N4CCC(F)CC4)n3)[nH]n2)cc1] |
| 65 | 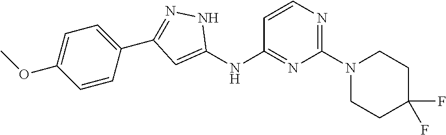 [SMILES: COc1ccc(-c2cc(Nc3ccnc(N4CCC(F)(F)CC4)n3)[nH]n2)cc1] |
| 66 | 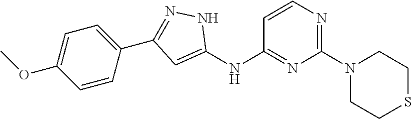 [SMILES: COc1ccc(-c2cc(Nc3ccnc(N4CCSCC4)n3)[nH]n2)cc1] |
| 67 | 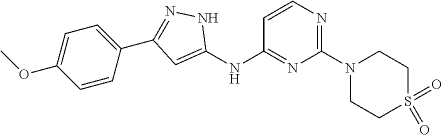 [SMILES: COc1ccc(-c2cc(Nc3ccnc(N4CCS(=O)(=O)CC4)n3)[nH]n2)cc1] |
| 68 | 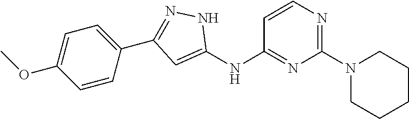 [SMILES: COc1ccc(-c2cc(Nc3ccnc(N4CCCCC4)n3)[nH]n2)cc1] |
| 69 | 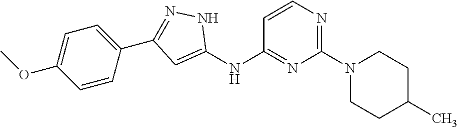 |
| 70 | 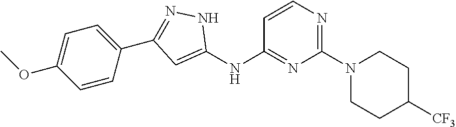 [SMILES: COc1ccc(-c2cc(Nc3ccnc(N4CCC(C(F)(F)F)CC4)n3)[nH]n2)cc1] |
| 71 | 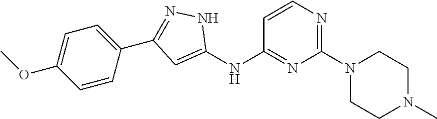 |
| 72 | 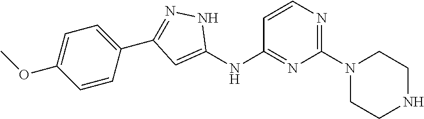 [SMILES: COc1ccc(-c2cc(Nc3ccnc(N4CCNCC4)n3)[nH]n2)cc1] |
| 73 | 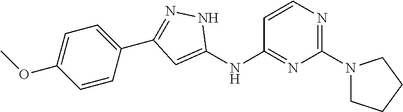 [SMILES: COc1ccc(-c2cc(Nc3ccnc(N4CCCC4)n3)[nH]n2)cc1] |
| 77 | 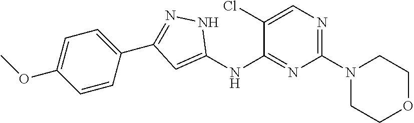 [SMILES: COc1ccc(-c2cc(Nc3nc(N4CCOCC4)ncc3Cl)[nH]n2)cc1] |
| 78 | 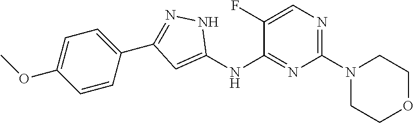 [SMILES: COc1ccc(-c2cc(Nc3nc(N4CCOCC4)ncc3F)[nH]n2)cc1] |
| 79 | 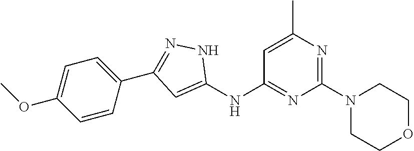 [SMILES: COc1ccc(-c2cc(Nc3cc(C)nc(N4CCOCC4)n3)[nH]n2)cc1] |
| 80 | 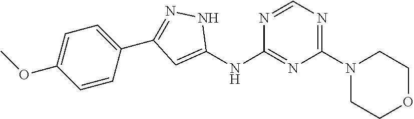 [SMILES: COc1ccc(-c2cc(Nc3ncnc(N4CCOCC4)n3)[nH]n2)cc1] |
| 81 | 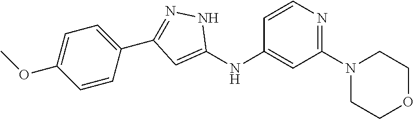 [SMILES: COc1ccc(-c2cc(Nc3ccnc(N4CCOCC4)c3)[nH]n2)cc1] |
| 82 | 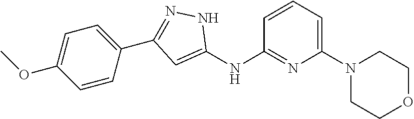 [SMILES: COc1ccc(-c2cc(Nc3cccc(N4CCOCC4)n3)[nH]n2)cc1] |
| 83 | 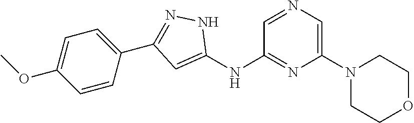 [SMILES: COc1ccc(-c2cc(Nc3cncc(N4CCOCC4)n3)[nH]n2)cc1] |
| 84 | 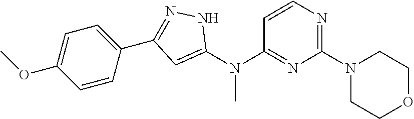 [SMILES: COc1ccc(-c2cc(N(C)c3ccnc(N4CCOCC4)n3)[nH]n2)cc1] |
| 85 | 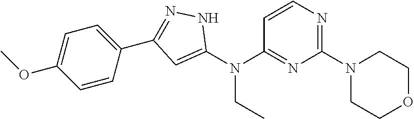 [SMILES: CCN(c1ccnc(N2CCOCC2)n1)c1cc(-c2ccc(OC)cc2)n[nH]1] |
| 86a | 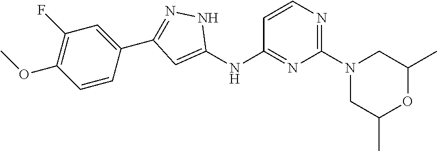 |
| 86b | 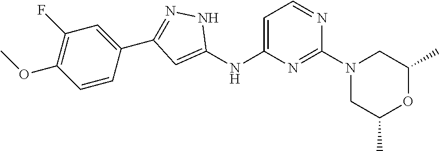 |
| 87 | 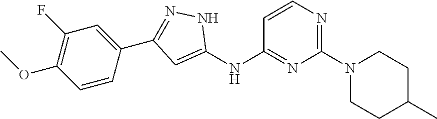 |
| 88 | 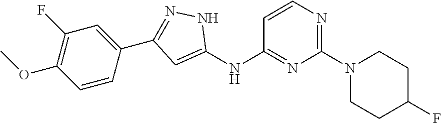 [SMILES: COc1ccc(-c2cc(Nc3ccnc(N4CCC(F)CC4)n3)[nH]n2)cc1F] |
| 89 | 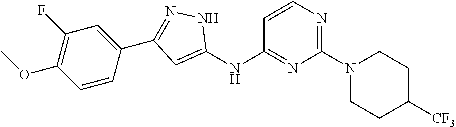 [SMILES: COc1ccc(-c2cc(Nc3ccnc(N4CCC(C(F)(F)F)CC4)n3)[nH]n2)cc1F] |
| 90 | 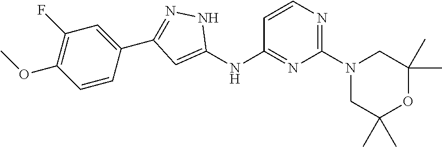 |
| 91 | 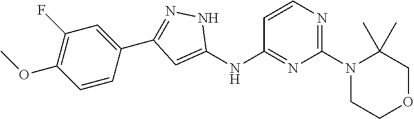 [SMILES: COc1ccc(-c2cc(Nc3ccnc(N4CCOCC4(C)C)n3)[nH]n2)cc1F] |
| 92 | 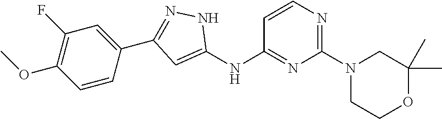 |
| 93 | 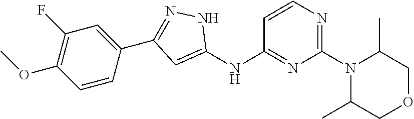 [SMILES: COc1ccc(-c2cc(Nc3ccnc(N4C(C)COCC4C)n3)[nH]n2)cc1F] |
| 94 | 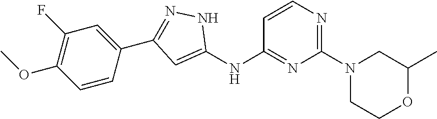 |
| 95 | 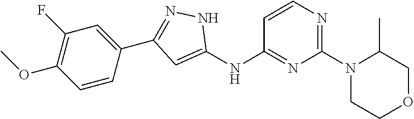 [SMILES: COc1ccc(-c2cc(Nc3ccnc(N4CCOCC4C)n3)[nH]n2)cc1F] |
| 96 | 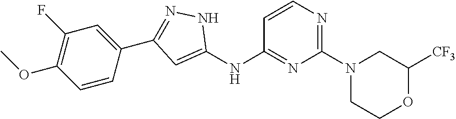 [SMILES: COc1ccc(-c2cc(Nc3ccnc(N4CCOC(C(F)(F)F)C4)n3)[nH]n2)cc1F] |
| 97 | 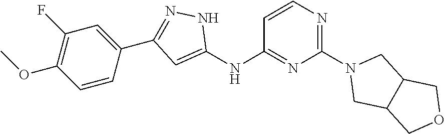 [SMILES: COc1ccc(-c2cc(Nc3ccnc(N4CC5COCC5C4)n3)[nH]n2)cc1F] |
| 98 | 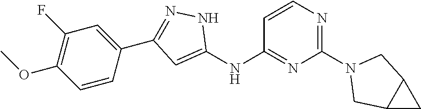 [SMILES: COc1ccc(-c2cc(Nc3ccnc(N4CC5CC5C4)n3)[nH]n2)cc1F] |
| 99 | 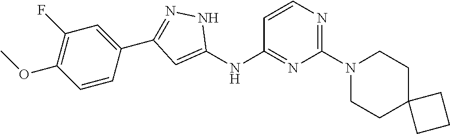 [SMILES: COc1ccc(-c2cc(Nc3ccnc(N4CCC5(CCC5)CC4)n3)[nH]n2)cc1F] |
| 100 | 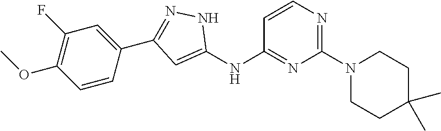 |
| 101 | 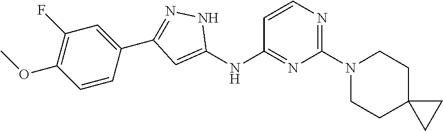 [SMILES: COc1ccc(-c2cc(Nc3ccnc(N4CCC5(CC4)CC5)n3)[nH]n2)cc1F] |
| 102 | 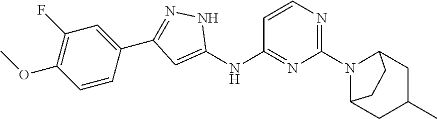 |
| 103 | 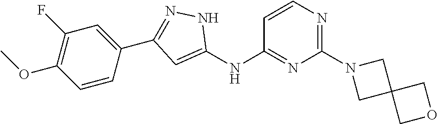 [SMILES: COc1ccc(-c2cc(Nc3ccnc(N4CC5(COC5)C4)n3)[nH]n2)cc1F] |
| 104 | 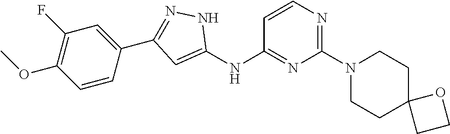 [SMILES: COc1ccc(-c2cc(Nc3ccnc(N4CCC5(CCO5)CC4)n3)[nH]n2)cc1F] |
| 105 | 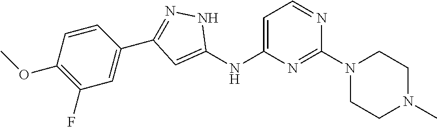 |
| 106 | 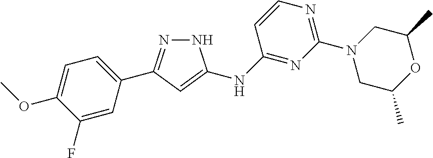 |
| 107 | 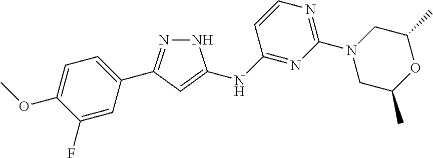 |
| 108 | 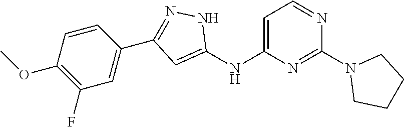 [SMILES: COc1ccc(-c2cc(Nc3ccnc(N4CCCC4)n3)[nH]n2)cc1F] |
| 109 | 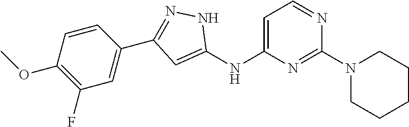 [SMILES: COc1ccc(-c2cc(Nc3ccnc(N4CCCCC4)n3)[nH]n2)cc1F] |
| 110 | 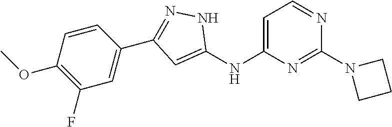 [SMILES: COc1ccc(-c2cc(Nc3ccnc(N4CCC4)n3)[nH]n2)cc1F] |
| 111 | 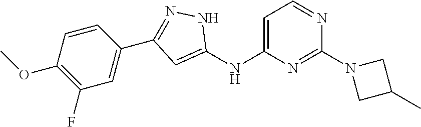 |
| 112 | 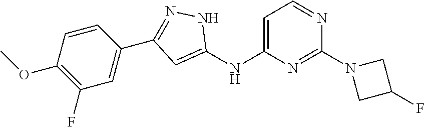 [SMILES: COc1ccc(-c2cc(Nc3ccnc(N4CC(F)C4)n3)[nH]n2)cc1F] |
| 113 | 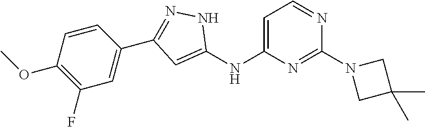 |
| 114 | 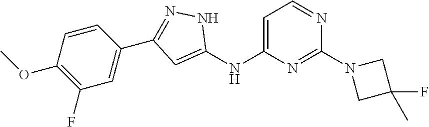 [SMILES: COc1ccc(-c2cc(Nc3ccnc(N4CC(C)(F)C4)n3)[nH]n2)cc1F] |
| 115 | 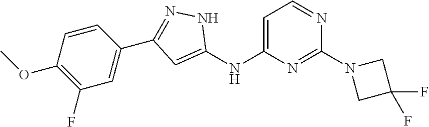 [SMILES: COc1ccc(-c2cc(Nc3ccnc(N4CC(F)(F)C4)n3)[nH]n2)cc1F] |
| 116 | 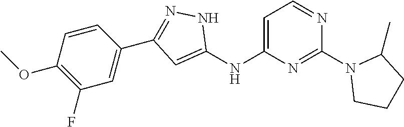 [SMILES: COc1ccc(-c2cc(Nc3ccnc(N4CCCC4C)n3)[nH]n2)cc1F] |
| 117 | 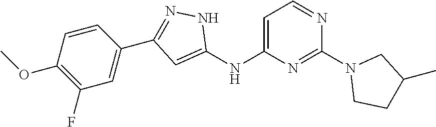 |
| 118 | 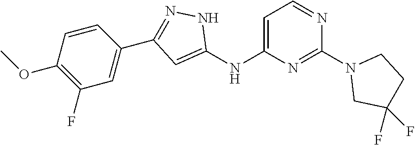 [SMILES: COc1ccc(-c2cc(Nc3ccnc(N4CCC(F)(F)C4)n3)[nH]n2)cc1F] |
| 119 | 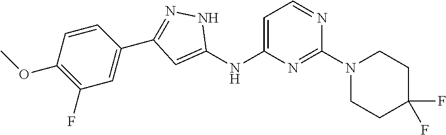 [SMILES: COc1ccc(-c2cc(Nc3ccnc(N4CCC(F)(F)CC4)n3)[nH]n2)cc1F] |
| 120 | 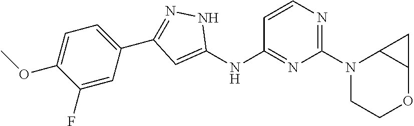 [SMILES: COc1ccc(-c2cc(Nc3ccnc(N4CCOC5CC54)n3)[nH]n2)cc1F] |
| 121 | 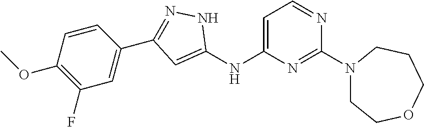 [SMILES: COc1ccc(-c2cc(Nc3ccnc(N4CCCOCC4)n3)[nH]n2)cc1F] |
| 122 | 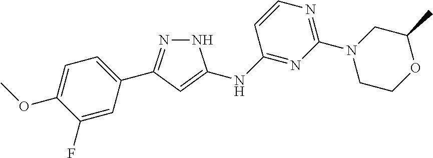 |
| 123 | 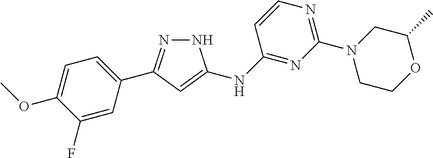 |
| 124 | 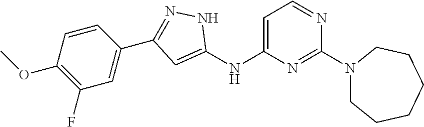 [SMILES: COc1ccc(-c2cc(Nc3ccnc(N4CCCCCC4)n3)[nH]n2)cc1F] |
| 125 | 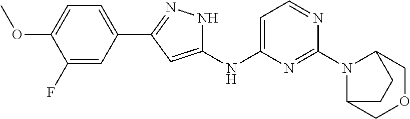 [SMILES: COc1ccc(-c2cc(Nc3ccnc(N4C5CCC4COC5)n3)[nH]n2)cc1F] |
| 126 | 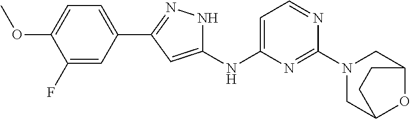 [SMILES: COc1ccc(-c2cc(Nc3ccnc(N4CC5CCC(C4)O5)n3)[nH]n2)cc1F] |
| 127 | 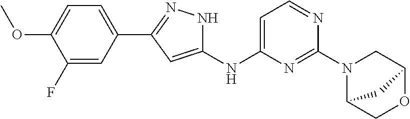 [SMILES: COc1ccc(-c2cc(Nc3ccnc(N4C[C@@H]5C[C@H]4CO5)n3)[nH]n2)cc1F] |
| 128 | 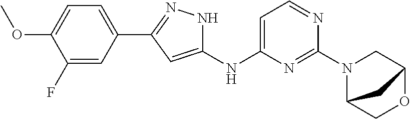 [SMILES: COc1ccc(-c2cc(Nc3ccnc(N4C[C@H]5C[C@@H]4CO5)n3)[nH]n2)cc1F] |
| 129a | 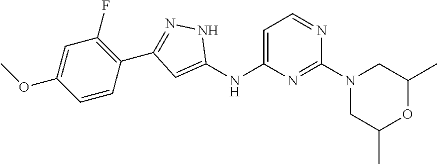 |
| 129b | 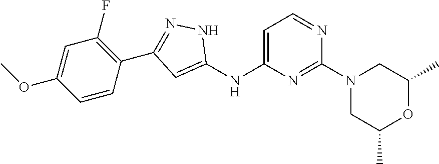 |
| 130 | 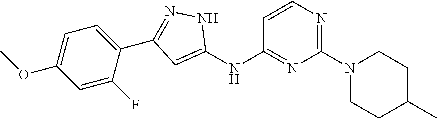 |
| 131 | 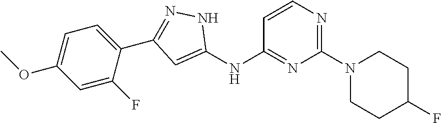 [SMILES: COc1ccc(-c2cc(Nc3ccnc(N4CCC(F)CC4)n3)[nH]n2)c(F)c1] |
| 132 | 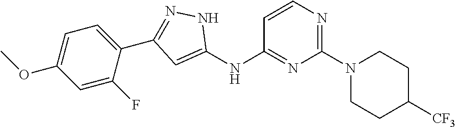 [SMILES: COc1ccc(-c2cc(Nc3ccnc(N4CCC(C(F)(F)F)CC4)n3)[nH]n2)c(F)c1] |
| 133 | 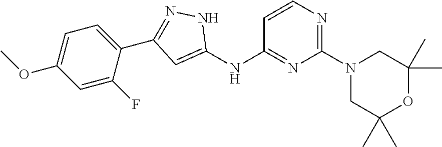 |
| 134 | 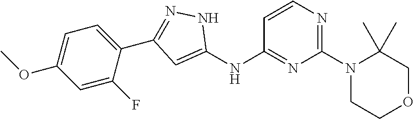 [SMILES: COc1ccc(-c2cc(Nc3ccnc(N4CCOCC4(C)C)n3)[nH]n2)c(F)c1] |
| 135 | 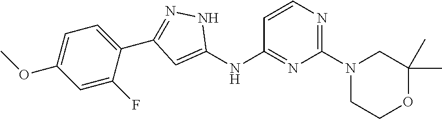 |
| 136 | 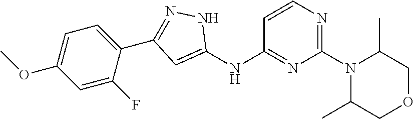 [SMILES: COc1ccc(-c2cc(Nc3ccnc(N4C(C)COCC4C)n3)[nH]n2)c(F)c1] |
| 137 | 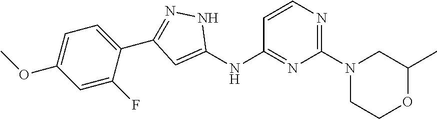 |
| 138 | 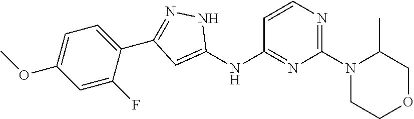 [SMILES: COc1ccc(-c2cc(Nc3ccnc(N4CCOCC4C)n3)[nH]n2)c(F)c1] |
| 139 | 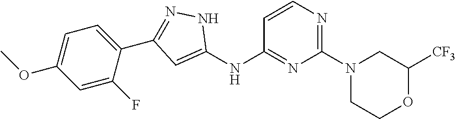 [SMILES: COc1ccc(-c2cc(Nc3ccnc(N4CCOC(C(F)(F)F)C4)n3)[nH]n2)c(F)c1] |
| 140 | 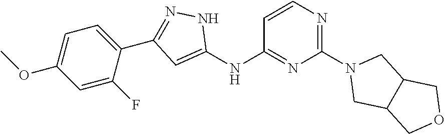 [SMILES: COc1ccc(-c2cc(Nc3ccnc(N4CC5COCC5C4)n3)[nH]n2)c(F)c1] |
| 141 | 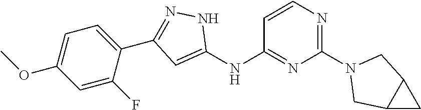 [SMILES: COc1ccc(-c2cc(Nc3ccnc(N4CC5CC5C4)n3)[nH]n2)c(F)c1] |
| 142 | 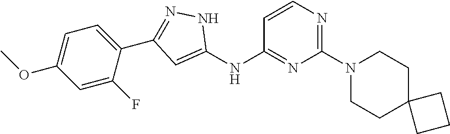 [SMILES: COc1ccc(-c2cc(Nc3ccnc(N4CCC5(CCC5)CC4)n3)[nH]n2)c(F)c1] |
| 143 | 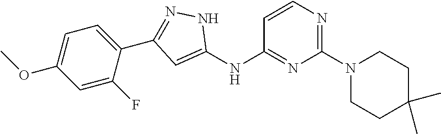 |
| 144 | 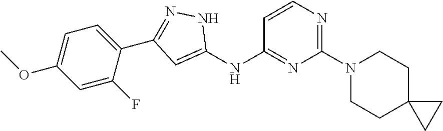 [SMILES: COc1ccc(-c2cc(Nc3ccnc(N4CCC5(CC4)CC5)n3)[nH]n2)c(F)c1] |
| 145 | 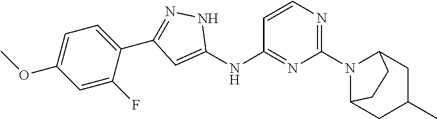 |
| 146 | 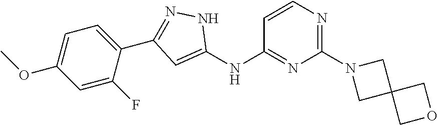 [SMILES: COc1ccc(-c2cc(Nc3ccnc(N4CC5(COC5)C4)n3)[nH]n2)c(F)c1] |
| 147 | 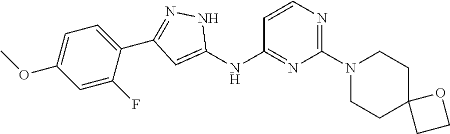 [SMILES: COc1ccc(-c2cc(Nc3ccnc(N4CCC5(CCO5)CC4)n3)[nH]n2)c(F)c1] |
| 148 | 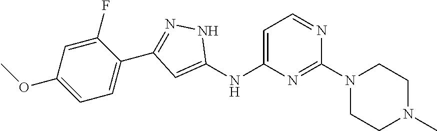 |
| 149 | 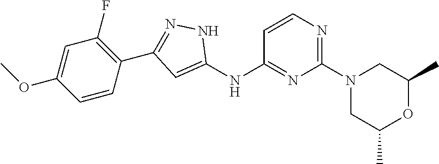 |
| 150 | 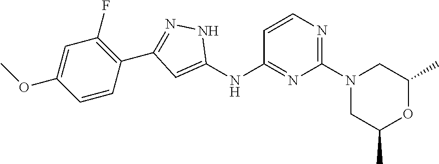 |
| 151 | 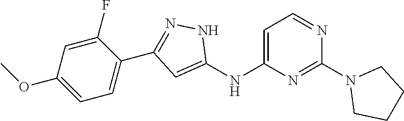 [SMILES: COc1ccc(-c2cc(Nc3ccnc(N4CCCC4)n3)[nH]n2)c(F)c1] |
| 152 | 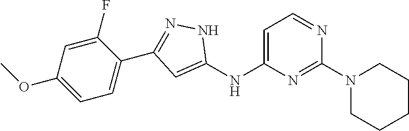 [SMILES: COc1ccc(-c2cc(Nc3ccnc(N4CCCCC4)n3)[nH]n2)c(F)c1] |
| 153 | 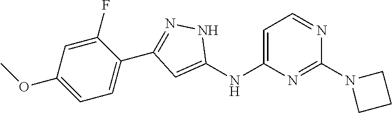 [SMILES: COc1ccc(-c2cc(Nc3ccnc(N4CCC4)n3)[nH]n2)c(F)c1] |
| 154 | 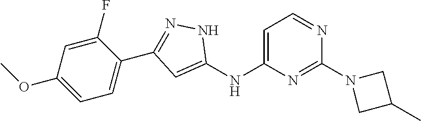 |
| 155 | 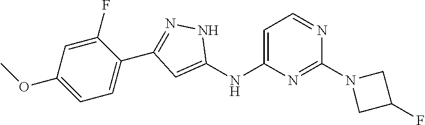 [SMILES: COc1ccc(-c2cc(Nc3ccnc(N4CC(F)C4)n3)[nH]n2)c(F)c1] |
| 156 | 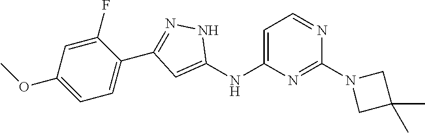 |
| 157 | 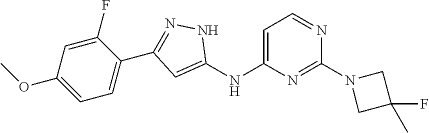 [SMILES: COc1ccc(-c2cc(Nc3ccnc(N4CC(C)(F)C4)n3)[nH]n2)c(F)c1] |
| 158 | 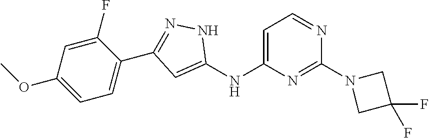 [SMILES: COc1ccc(-c2cc(Nc3ccnc(N4CC(F)(F)C4)n3)[nH]n2)c(F)c1] |
| 159 | 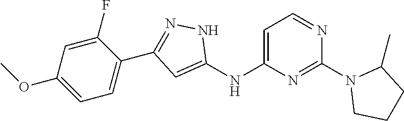 [SMILES: COc1ccc(-c2cc(Nc3ccnc(N4CCCC4C)n3)[nH]n2)c(F)c1] |
| 160 | 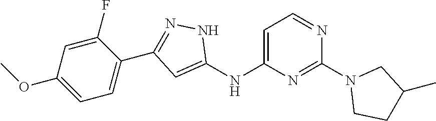 |
| 161 | 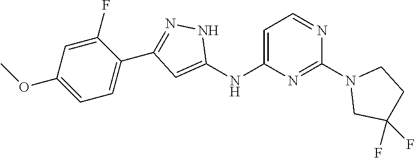 [SMILES: COc1ccc(-c2cc(Nc3ccnc(N4CCC(F)(F)C4)n3)[nH]n2)c(F)c1] |
| 162 | 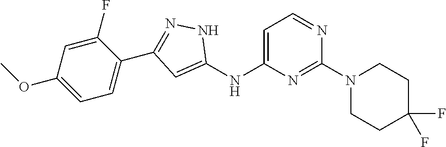 [SMILES: COc1ccc(-c2cc(Nc3ccnc(N4CCC(F)(F)CC4)n3)[nH]n2)c(F)c1] |
| 163 | 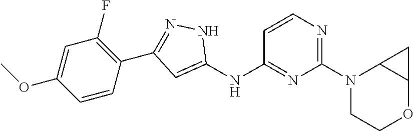 [SMILES: COc1ccc(-c2cc(Nc3ccnc(N4CCOC5CC54)n3)[nH]n2)c(F)c1] |
| 164 | 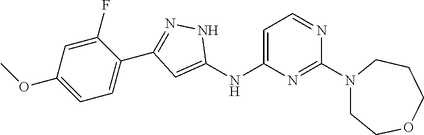 [SMILES: COc1ccc(-c2cc(Nc3ccnc(N4CCCOCC4)n3)[nH]n2)c(F)c1] |
| 165 | 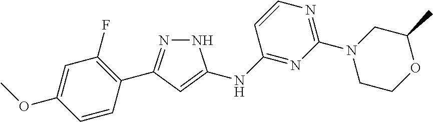 |
| 166 | 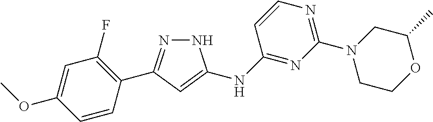 |
| 167 | 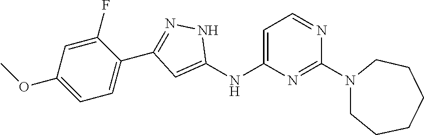 [SMILES: COc1ccc(-c2cc(Nc3ccnc(N4CCCCCC4)n3)[nH]n2)c(F)c1] |
| 168 | 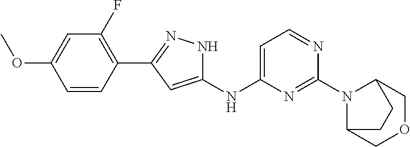 [SMILES: COc1ccc(-c2cc(Nc3ccnc(N4C5CCC4COC5)n3)[nH]n2)c(F)c1] |
| 169 | 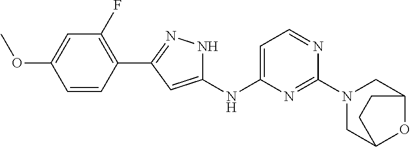 [SMILES: COc1ccc(-c2cc(Nc3ccnc(N4CC5CCC(C4)O5)n3)[nH]n2)c(F)c1] |
| 170 | 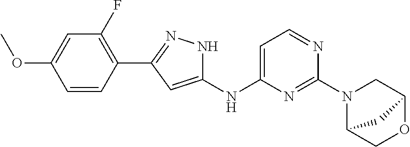 [SMILES: COc1ccc(-c2cc(Nc3ccnc(N4C[C@@H]5C[C@H]4CO5)n3)[nH]n2)c(F)c1] |
| 171 | 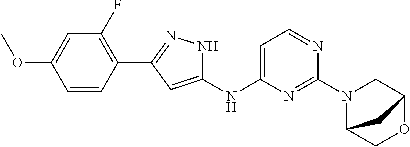 [SMILES: COc1ccc(-c2cc(Nc3ccnc(N4C[C@H]5C[C@@H]4CO5)n3)[nH]n2)c(F)c1] |
| 174 | 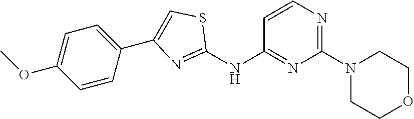 [SMILES: COc1ccc(-c2csc(Nc3ccnc(N4CCOCC4)n3)n2)cc1] |
| 177 | 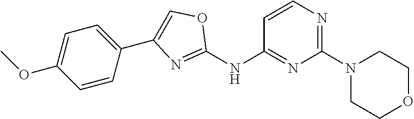 [SMILES: COc1ccc(-c2coc(Nc3ccnc(N4CCOCC4)n3)n2)cc1] |
| 181 | 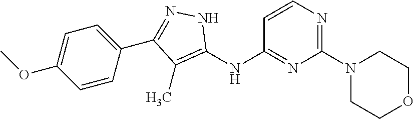 [SMILES: COc1ccc(-c2n[nH]c(Nc3ccnc(N4CCOCC4)n3)c2C)cc1] |
| 184 | 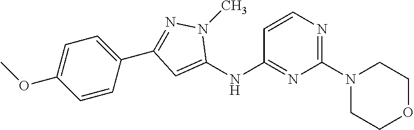 [SMILES: COc1ccc(-c2cc(Nc3ccnc(N4CCOCC4)n3)n(C)n2)cc1] |
| 187 | 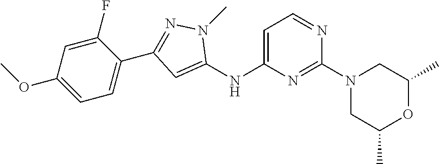 |
| 190 | 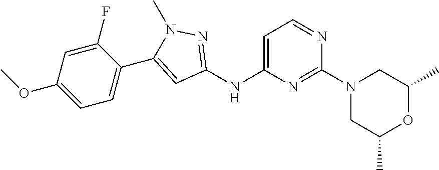 |
| 191a | 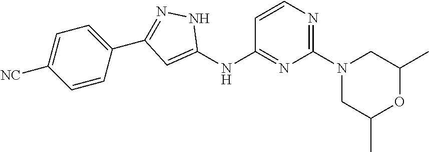 [SMILES: CC1CN(c2nccc(Nc3cc(-c4ccc(C#N)cc4)n[nH]3)n2)CC(C)O1] |
| 191b | 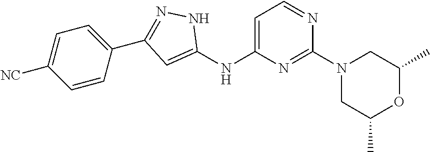 [SMILES: C[C@@H]1CN(c2nccc(Nc3cc(-c4ccc(C#N)cc4)n[nH]3)n2)C[C@H](C)O1] |
| 192a | 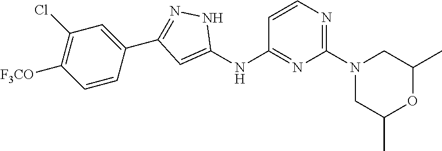 [SMILES: CC1CN(c2nccc(Nc3cc(-c4ccc(OC(F)(F)F)c(Cl)c4)n[nH]3)n2)CC(C)O1] |
| 192b | 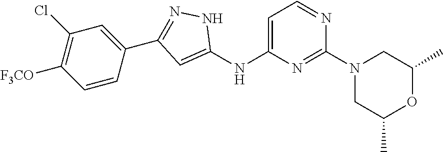 [SMILES: C[C@@H]1CN(c2nccc(Nc3cc(-c4ccc(OC(F)(F)F)c(Cl)c4)n[nH]3)n2)C[C@H](C)O1] |
| 193 | 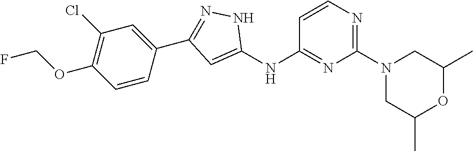 [SMILES: CC1CN(c2nccc(Nc3cc(-c4ccc(OCF)c(Cl)c4)n[nH]3)n2)CC(C)O1] |
| 194 | 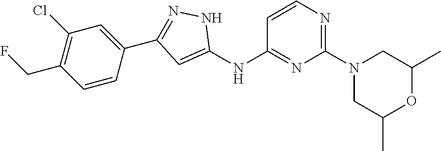 [SMILES: CC1CN(c2nccc(Nc3cc(-c4ccc(CF)c(Cl)c4)n[nH]3)n2)CC(C)O1] |
| 195 | 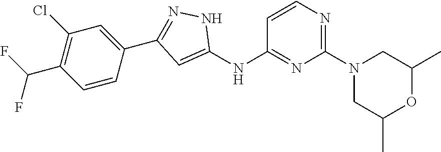 [SMILES: CC1CN(c2nccc(Nc3cc(-c4ccc(C(F)F)c(Cl)c4)n[nH]3)n2)CC(C)O1] |
| 196 | 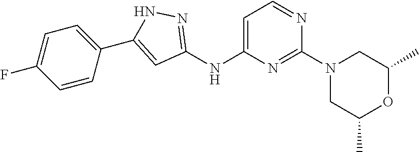 [SMILES: C[C@@H]1CN(c2nccc(Nc3cc(-c4ccc(F)cc4)[nH]n3)n2)C[C@H](C)O1] |
| 197 | 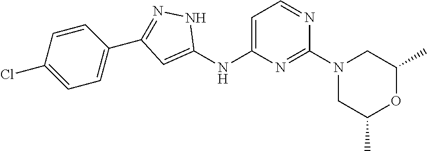 [SMILES: C[C@@H]1CN(c2nccc(Nc3cc(-c4ccc(Cl)cc4)n[nH]3)n2)C[C@H](C)O1] |
| 198 | 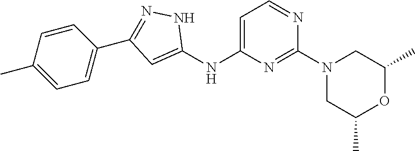 |
| 199 | 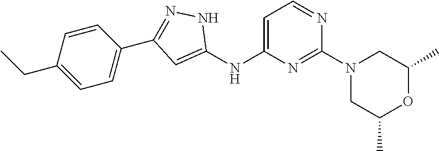 [SMILES: CCc1ccc(-c2cc(Nc3ccnc(N4C[C@@H](C)O[C@@H](C)C4)n3)[nH]n2)cc1] |
| 200 | 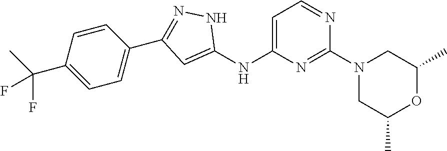 [SMILES: C[C@@H]1CN(c2nccc(Nc3cc(-c4ccc(C(C)(F)F)cc4)n[nH]3)n2)C[C@H](C)O1] |
| 201 | 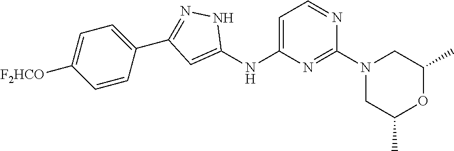 [SMILES: C[C@@H]1CN(c2nccc(Nc3cc(-c4ccc(OC(F)F)cc4)n[nH]3)n2)C[C@H](C)O1] |
| 202 | 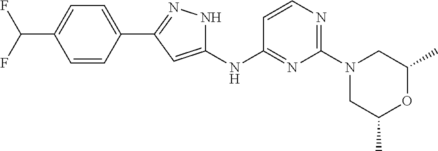 [SMILES: C[C@@H]1CN(c2nccc(Nc3cc(-c4ccc(C(F)F)cc4)n[nH]3)n2)C[C@H](C)O1] |
| 203 | 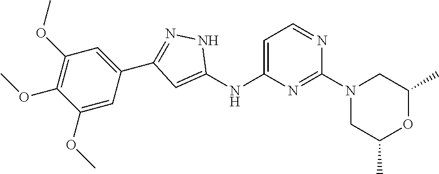 |
| 204 | 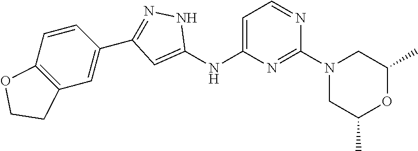 [SMILES: C[C@@H]1CN(c2nccc(Nc3cc(-c4ccc5c(c4)CCO5)n[nH]3)n2)C[C@H](C)O1] |
| 205 | 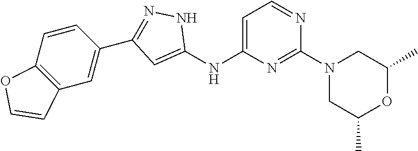 [SMILES: C[C@@H]1CN(c2nccc(Nc3cc(-c4ccc5occc5c4)n[nH]3)n2)C[C@H](C)O1] |
| 206 | 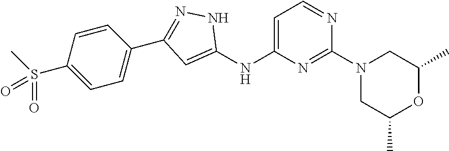 [SMILES: C[C@@H]1CN(c2nccc(Nc3cc(-c4ccc(S(C)(=O)=O)cc4)n[nH]3)n2)C[C@H](C)O1] |
| 207 | 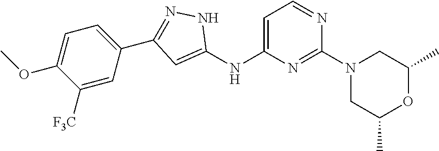 |
| 208 | 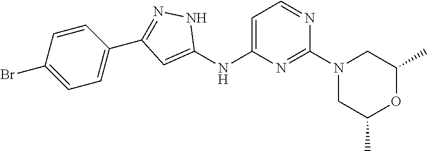 [SMILES: C[C@@H]1CN(c2nccc(Nc3cc(-c4ccc(Br)cc4)n[nH]3)n2)C[C@H](C)O1] |
| 209 | 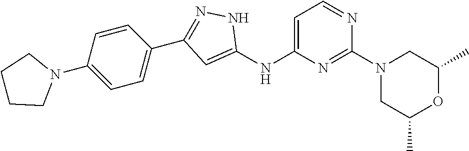 [SMILES: C[C@@H]1CN(c2nccc(Nc3cc(-c4ccc(N5CCCC5)cc4)n[nH]3)n2)C[C@H](C)O1] |
| 210 | 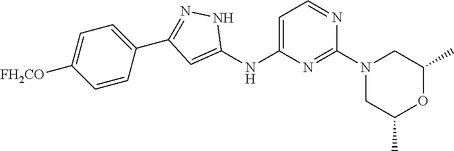 |
| 211 | 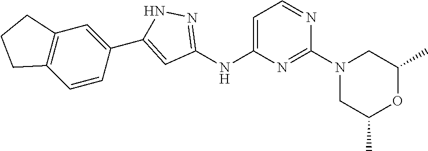 [SMILES: C[C@@H]1CN(c2nccc(Nc3cc(-c4ccc5c(c4)CCC5)[nH]n3)n2)C[C@H](C)O1] |
| 212 | 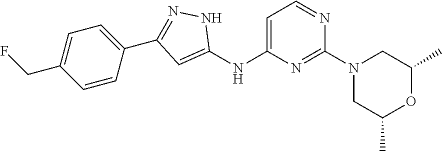 [SMILES: C[C@@H]1CN(c2nccc(Nc3cc(-c4ccc(CF)cc4)n[nH]3)n2)C[C@H](C)O1] |
| 213 | 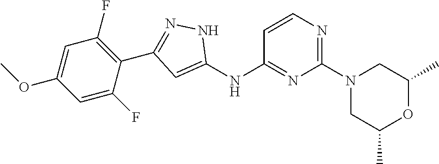 |
| 214 | 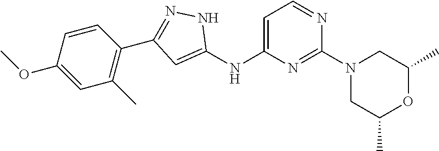 |
| 215 | 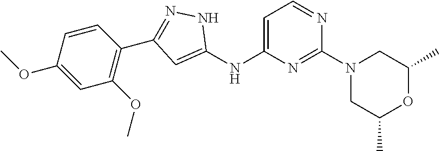 |
| 216 | 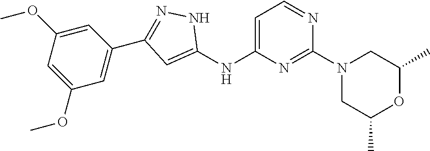 |
| 217 | 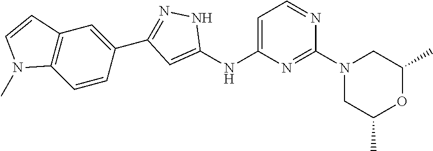 |
| 218 | 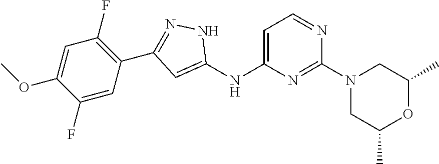 |
| 219 | 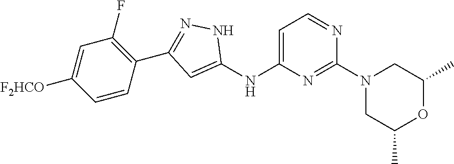 [SMILES: C[C@@H]1CN(c2nccc(Nc3cc(-c4ccc(OC(F)F)cc4F)n[nH]3)n2)C[C@H](C)O1] |
| 220a | 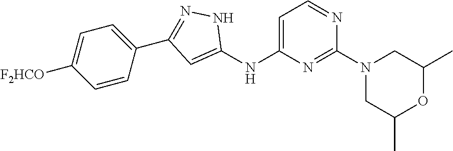 [SMILES: CC1CN(c2nccc(Nc3cc(-c4ccc(OC(F)F)cc4)n[nH]3)n2)CC(C)O1] |
| 220b | 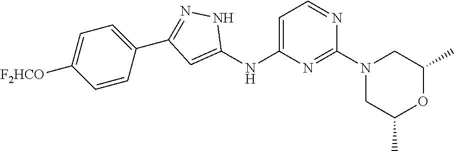 [SMILES: C[C@@H]1CN(c2nccc(Nc3cc(-c4ccc(OC(F)F)cc4)n[nH]3)n2)C[C@H](C)O1] |
| 221 | 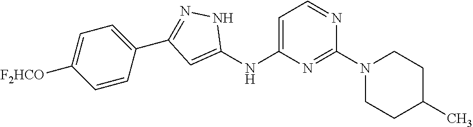 [SMILES: CC1CCN(c2nccc(Nc3cc(-c4ccc(OC(F)F)cc4)n[nH]3)n2)CC1] |
| 222 | 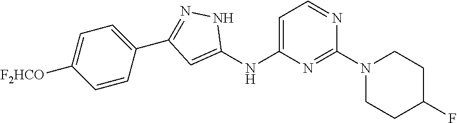 [SMILES: FC1CCN(c2nccc(Nc3cc(-c4ccc(OC(F)F)cc4)n[nH]3)n2)CC1] |
| 223 | 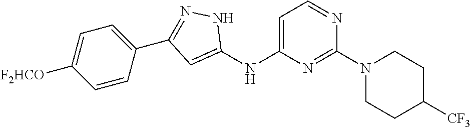 [SMILES: FC(F)Oc1ccc(-c2cc(Nc3ccnc(N4CCC(C(F)(F)F)CC4)n3)[nH]n2)cc1] |
| 224 | 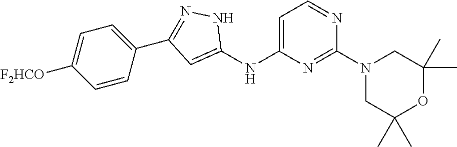 [SMILES: CC1(C)CN(c2nccc(Nc3cc(-c4ccc(OC(F)F)cc4)n[nH]3)n2)CC(C)(C)O1] |
| 225 | 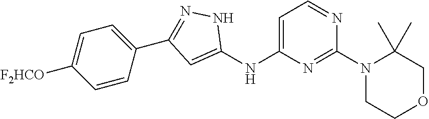 [SMILES: CC1(C)COCCN1c1nccc(Nc2cc(-c3ccc(OC(F)F)cc3)n[nH]2)n1] |
| 226 | 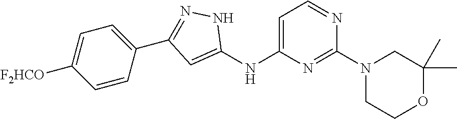 [SMILES: CC1(C)CN(c2nccc(Nc3cc(-c4ccc(OC(F)F)cc4)n[nH]3)n2)CCO1] |
| 227 | 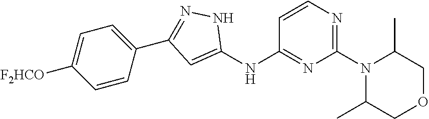 [SMILES: CC1COCC(C)N1c1nccc(Nc2cc(-c3ccc(OC(F)F)cc3)n[nH]2)n1] |
| 228 | 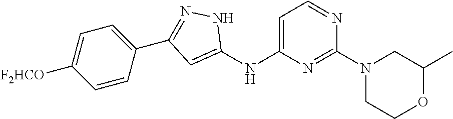 [SMILES: CC1CN(c2nccc(Nc3cc(-c4ccc(OC(F)F)cc4)n[nH]3)n2)CCO1] |
| 229 | 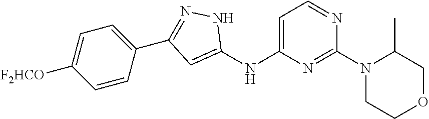 [SMILES: CC1COCCN1c1nccc(Nc2cc(-c3ccc(OC(F)F)cc3)n[nH]2)n1] |
| 230 | 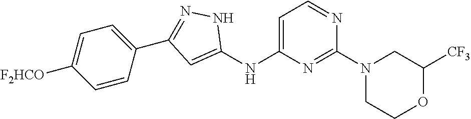 [SMILES: FC(F)Oc1ccc(-c2cc(Nc3ccnc(N4CCOC(C(F)(F)F)C4)n3)[nH]n2)cc1] |
| 231 | 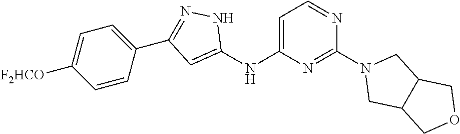 [SMILES: FC(F)Oc1ccc(-c2cc(Nc3ccnc(N4CC5COCC5C4)n3)[nH]n2)cc1] |
| 232 | 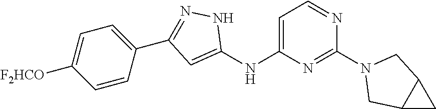 [SMILES: FC(F)Oc1ccc(-c2cc(Nc3ccnc(N4CC5CC5C4)n3)[nH]n2)cc1] |
| 233 | 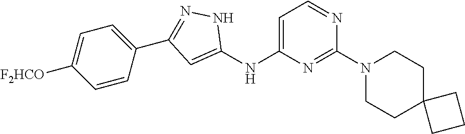 [SMILES: FC(F)Oc1ccc(-c2cc(Nc3ccnc(N4CCC5(CCC5)CC4)n3)[nH]n2)cc1] |
| 234 | 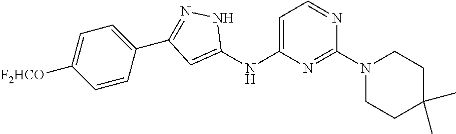 [SMILES: CC1(C)CCN(c2nccc(Nc3cc(-c4ccc(OC(F)F)cc4)n[nH]3)n2)CC1] |
| 235 | 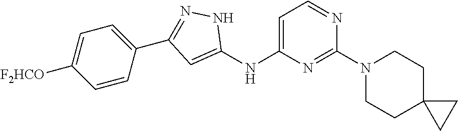 [SMILES: FC(F)Oc1ccc(-c2cc(Nc3ccnc(N4CCC5(CC4)CC5)n3)[nH]n2)cc1] |
| 236 | 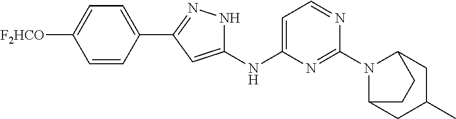 [SMILES: CC1CC2CCC(C1)N2c1nccc(Nc2cc(-c3ccc(OC(F)F)cc3)n[nH]2)n1] |
| 237 | 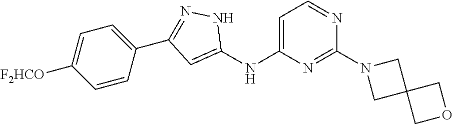 [SMILES: FC(F)Oc1ccc(-c2cc(Nc3ccnc(N4CC5(COC5)C4)n3)[nH]n2)cc1] |
| 238 | 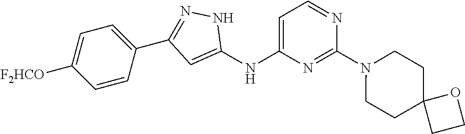 [SMILES: FC(F)Oc1ccc(-c2cc(Nc3ccnc(N4CCC5(CCO5)CC4)n3)[nH]n2)cc1] |
| 239 | 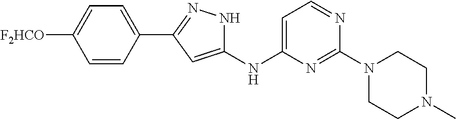 [SMILES: CN1CCN(c2nccc(Nc3cc(-c4ccc(OC(F)F)cc4)n[nH]3)n2)CC1] |
| 240 | 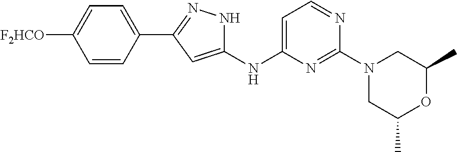 [SMILES: C[C@@H]1CN(c2nccc(Nc3cc(-c4ccc(OC(F)F)cc4)n[nH]3)n2)C[C@@H](C)O1] |
| 241 | 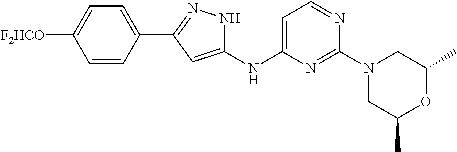 [SMILES: C[C@H]1CN(c2nccc(Nc3cc(-c4ccc(OC(F)F)cc4)n[nH]3)n2)C[C@H](C)O1] |
| 242 | 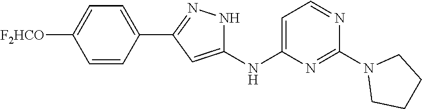 [SMILES: FC(F)Oc1ccc(-c2cc(Nc3ccnc(N4CCCC4)n3)[nH]n2)cc1] |
| 243 | 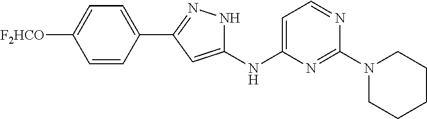 [SMILES: FC(F)Oc1ccc(-c2cc(Nc3ccnc(N4CCCCC4)n3)[nH]n2)cc1] |
| 244 | 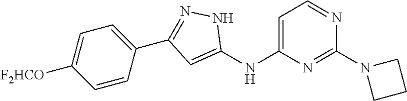 [SMILES: FC(F)Oc1ccc(-c2cc(Nc3ccnc(N4CCC4)n3)[nH]n2)cc1] |
| 245 | 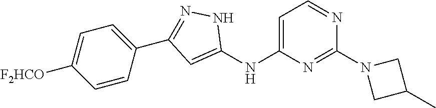 [SMILES: CC1CN(c2nccc(Nc3cc(-c4ccc(OC(F)F)cc4)n[nH]3)n2)C1] |
| 246 | 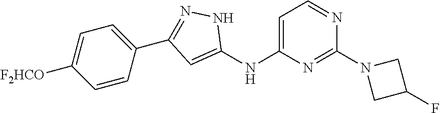 [SMILES: FC1CN(c2nccc(Nc3cc(-c4ccc(OC(F)F)cc4)n[nH]3)n2)C1] |
| 247 | 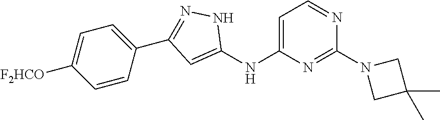 [SMILES: CC1(C)CN(c2nccc(Nc3cc(-c4ccc(OC(F)F)cc4)n[nH]3)n2)C1] |
| 248 | 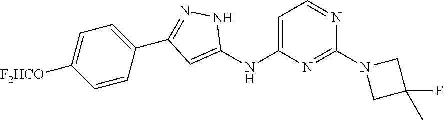 [SMILES: CC1(F)CN(c2nccc(Nc3cc(-c4ccc(OC(F)F)cc4)n[nH]3)n2)C1] |
| 249 | 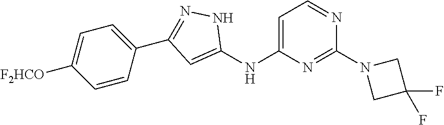 [SMILES: FC(F)Oc1ccc(-c2cc(Nc3ccnc(N4CC(F)(F)C4)n3)[nH]n2)cc1] |
| 250 | 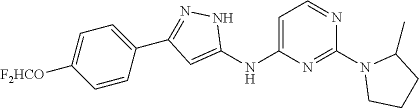 [SMILES: CC1CCCN1c1nccc(Nc2cc(-c3ccc(OC(F)F)cc3)n[nH]2)n1] |
| 251 | 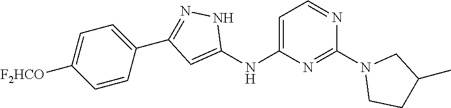 [SMILES: CC1CCN(c2nccc(Nc3cc(-c4ccc(OC(F)F)cc4)n[nH]3)n2)C1] |
| 252 | 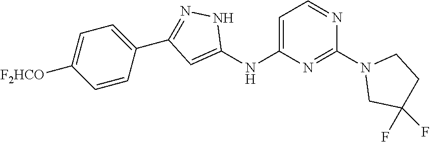 [SMILES: FC(F)Oc1ccc(-c2cc(Nc3ccnc(N4CCC(F)(F)C4)n3)[nH]n2)cc1] |
| 253 | 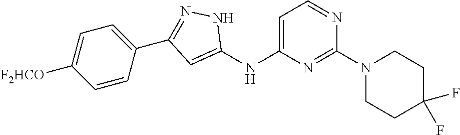 [SMILES: FC(F)Oc1ccc(-c2cc(Nc3ccnc(N4CCC(F)(F)CC4)n3)[nH]n2)cc1] |
| 254 | 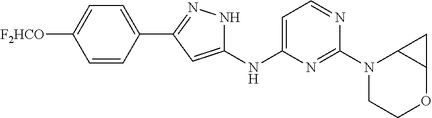 [SMILES: FC(F)Oc1ccc(-c2cc(Nc3ccnc(N4CCOC5CC54)n3)[nH]n2)cc1] |
| 255 | 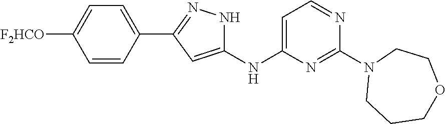 [SMILES: FC(F)Oc1ccc(-c2cc(Nc3ccnc(N4CCCOCC4)n3)[nH]n2)cc1] |
| 256 | 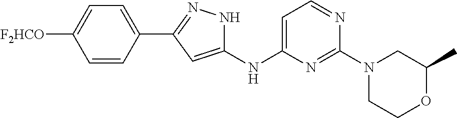 [SMILES: C[C@@H]1CN(c2nccc(Nc3cc(-c4ccc(OC(F)F)cc4)n[nH]3)n2)CCO1] |
| 257 | 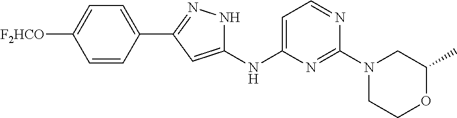 [SMILES: C[C@H]1CN(c2nccc(Nc3cc(-c4ccc(OC(F)F)cc4)n[nH]3)n2)CCO1] |
| 258 | 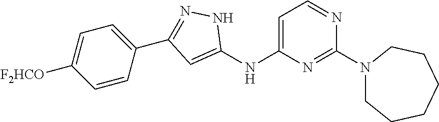 [SMILES: FC(F)Oc1ccc(-c2cc(Nc3ccnc(N4CCCCCC4)n3)[nH]n2)cc1] |
| 259 | 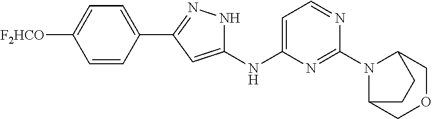 [SMILES: FC(F)Oc1ccc(-c2cc(Nc3ccnc(N4C5CCC4COC5)n3)[nH]n2)cc1] |
| 260 | 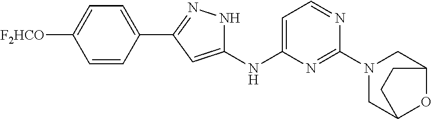 [SMILES: FC(F)Oc1ccc(-c2cc(Nc3ccnc(N4CC5CCC(C4)O5)n3)[nH]n2)cc1] |
| 261 | 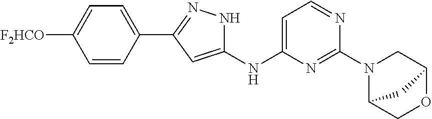 [SMILES: FC(F)Oc1ccc(-c2cc(Nc3ccnc(N4C[C@@H]5C[C@H]4CO5)n3)[nH]n2)cc1] |
| 262 | 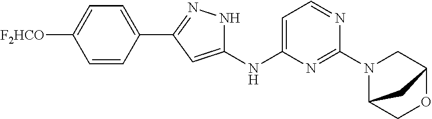 [SMILES: FC(F)Oc1ccc(-c2cc(Nc3ccnc(N4C[C@H]5C[C@@H]4CO5)n3)[nH]n2)cc1] |
| 263 | 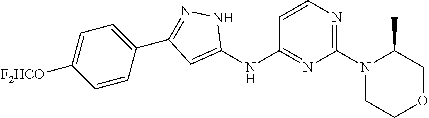 [SMILES: C[C@H]1COCCN1c1nccc(Nc2cc(-c3ccc(OC(F)F)cc3)n[nH]2)n1] |
| 264 | 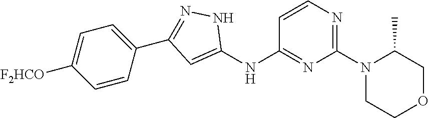 [SMILES: C[C@@H]1COCCN1c1nccc(Nc2cc(-c3ccc(OC(F)F)cc3)n[nH]2)n1] |
| 265 | 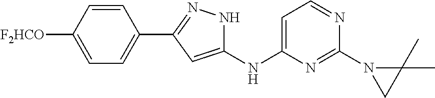 [SMILES: CC1(C)CN1c1nccc(Nc2cc(-c3ccc(OC(F)F)cc3)n[nH]2)n1] |
| 274 | 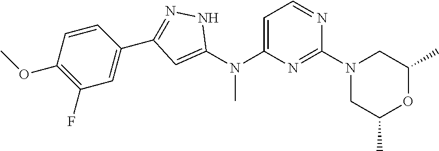 |
| 275 | 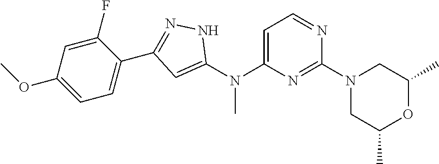 |
| 276 | 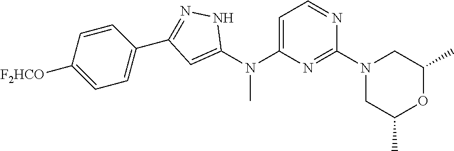 [SMILES: C[C@@H]1CN(c2nccc(N(C)c3cc(-c4ccc(OC(F)F)cc4)n[nH]3)n2)C[C@H](C)O1] |
| 277 | 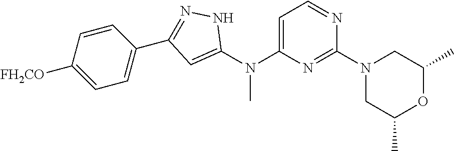 |
| 281 | 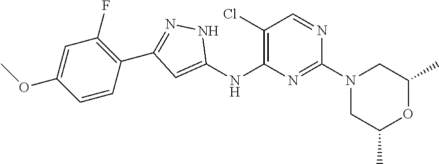 |
| 282 | 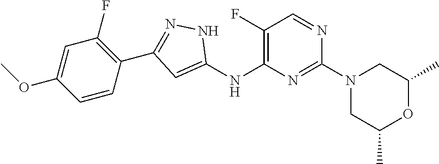 |
| 283 | 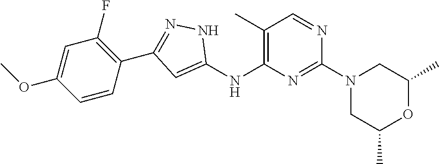 |
| 286 | 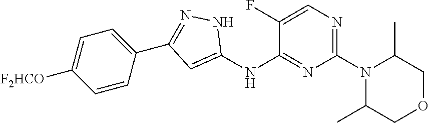 [SMILES: CC1COCC(C)N1c1ncc(F)c(Nc2cc(-c3ccc(OC(F)F)cc3)n[nH]2)n1] |
| 287 | 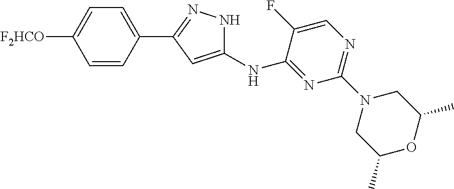 [SMILES: C[C@@H]1CN(c2ncc(F)c(Nc3cc(-c4ccc(OC(F)F)cc4)n[nH]3)n2)C[C@H](C)O1] |
| 288 | 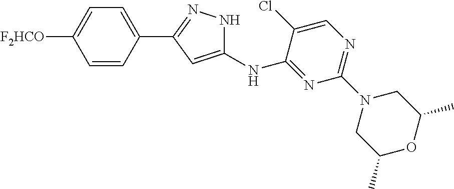 [SMILES: C[C@@H]1CN(c2ncc(Cl)c(Nc3cc(-c4ccc(OC(F)F)cc4)n[nH]3)n2)C[C@H](C)O1] |
| 289 | 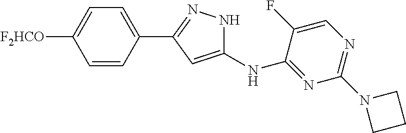 [SMILES: Fc1cnc(N2CCC2)nc1Nc1cc(-c2ccc(OC(F)F)cc2)n[nH]1] |
| 290 | 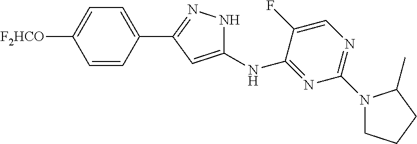 [SMILES: CC1CCCN1c1ncc(F)c(Nc2cc(-c3ccc(OC(F)F)cc3)n[nH]2)n1] |
| 291 | 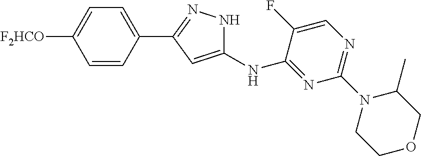 [SMILES: CC1COCCN1c1ncc(F)c(Nc2cc(-c3ccc(OC(F)F)cc3)n[nH]2)n1] |
| 292 | 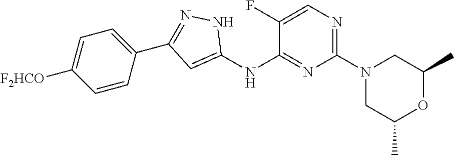 [SMILES: C[C@@H]1CN(c2ncc(F)c(Nc3cc(-c4ccc(OC(F)F)cc4)n[nH]3)n2)C[C@@H](C)O1] |
| 293 | 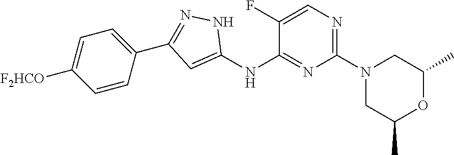 [SMILES: C[C@H]1CN(c2ncc(F)c(Nc3cc(-c4ccc(OC(F)F)cc4)n[nH]3)n2)C[C@H](C)O1] |
| 294 | 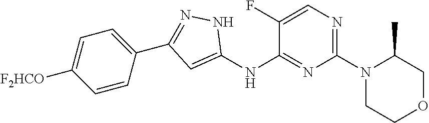 [SMILES: C[C@H]1COCCN1c1ncc(F)c(Nc2cc(-c3ccc(OC(F)F)cc3)n[nH]2)n1] |
| 295 | 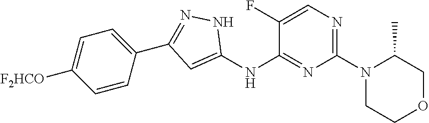 [SMILES: C[C@@H]1COCCN1c1ncc(F)c(Nc2cc(-c3ccc(OC(F)F)cc3)n[nH]2)n1] |
| 298 | 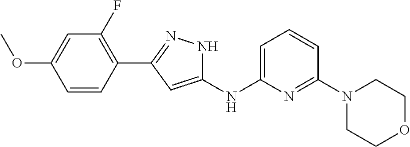 [SMILES: COc1ccc(-c2cc(Nc3cccc(N4CCOCC4)n3)[nH]n2)c(F)c1] |
| 299 | 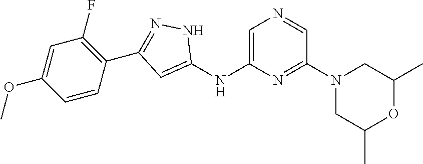 |
| 300 | 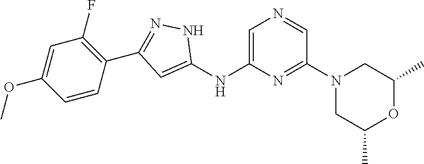 |
| 301 | 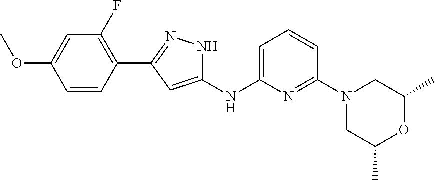 |
| 302 | 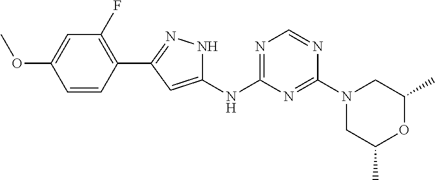 |
| 303 | 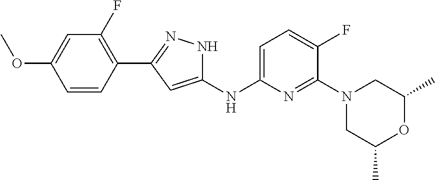 |
| 304 | 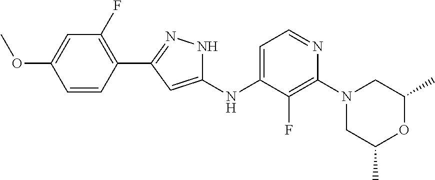 |
| 308 | 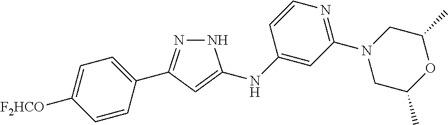 [SMILES: C[C@@H]1CN(c2cc(Nc3cc(-c4ccc(OC(F)F)cc4)n[nH]3)ccn2)C[C@H](C)O1] |
| 309 | 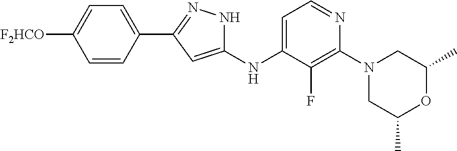 [SMILES: C[C@@H]1CN(c2nccc(Nc3cc(-c4ccc(OC(F)F)cc4)n[nH]3)c2F)C[C@H](C)O1] |
| 310 | 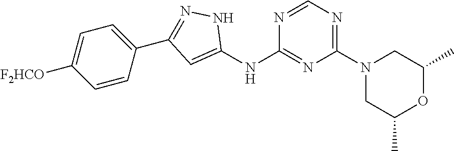 [SMILES: C[C@@H]1CN(c2ncnc(Nc3cc(-c4ccc(OC(F)F)cc4)n[nH]3)n2)C[C@H](C)O1] |
| 311 | 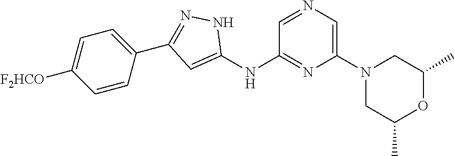 [SMILES: C[C@@H]1CN(c2cncc(Nc3cc(-c4ccc(OC(F)F)cc4)n[nH]3)n2)C[C@H](C)O1] |
| 315 | 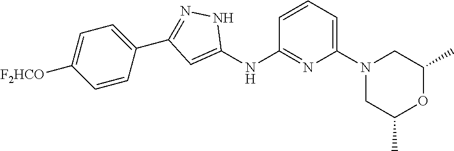 [SMILES: C[C@@H]1CN(c2cccc(Nc3cc(-c4ccc(OC(F)F)cc4)n[nH]3)n2)C[C@H](C)O1] |
| 316 | 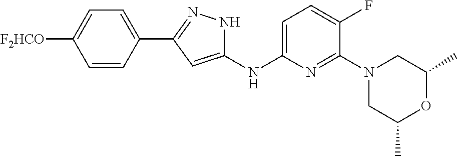 [SMILES: C[C@@H]1CN(c2nc(Nc3cc(-c4ccc(OC(F)F)cc4)n[nH]3)ccc2F)C[C@H](C)O1] |
| 317 | 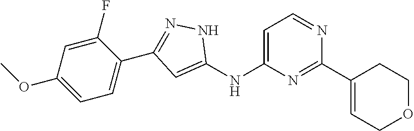 [SMILES: COc1ccc(-c2cc(Nc3ccnc(C4=CCOCC4)n3)[nH]n2)c(F)c1] |
| 318 | 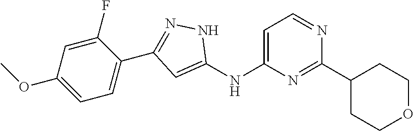 [SMILES: COc1ccc(-c2cc(Nc3ccnc(C4CCOCC4)n3)[nH]n2)c(F)c1] |

This disclosure includes all suitable isotopic variations of a compound of the disclosure. An isotopic variation of a compound of the invention is defined as one in which at least one atom is replaced by an atom having the same atomic number but an atomic mass different from the atomic mass usually or predominantly found in nature. Examples of isotopes that can be incorporated into a compound of the invention include isotopes of hydrogen, carbon, nitrogen, oxygen, phosphorus, sulfur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I, respectively. Accordingly, recitation of “hydrogen” or “H” should be understood to encompass ¹H (protium), ²H (deuterium), and ³H (tritium) unless otherwise specified. Certain isotopic variations of a compound of the invention, for example, those in which one or more radioactive isotopes such as ³H or ¹⁴C are incorporated, are useful in drug and/or substrate tissue distribution studies. Tritiated and carbon-14, i.e., ¹⁴C, isotopes are particularly preferred for their ease of preparation and detectability. Further, substitution with isotopes such as deuterium may afford certain therapeutic advantages resulting from greater metabolic stability, for example, increased in vivo half-life or reduced dosage requirements and hence may be preferred in some circumstances. Such variants may also have advantageous optical properties arising, for example, from changes to vibrational modes due to the heavier isotope. Isotopic variations of a compound of the invention can generally be prepared by conventional procedures known by a person skilled in the art such as by the illustrative methods or by the preparations described in the examples hereafter using appropriate isotopic variations of suitable reagents.

In another aspect, the present disclosure provides a pharmaceutical composition comprising a compound disclosed herein and a pharmaceutically acceptable excipient.

In yet another aspect, the present disclosure provides methods of treating TACC mediated diseases or disorders in a subject comprising administering a compound disclosed herein or a pharmaceutically acceptable salt thereof to the subject.

In certain embodiments, the TACC is TACC1. In other embodiments, the TACC is TACC2. In other preferred embodiments, the TACC is TACC3.

In certain embodiments, the TACC mediated disease or disorder is cancer. In certain embodiments, the cancer is breast cancer, colon cancer, melanoma cancer, lung cancer, central nervous system cancer, ovarian cancer, leukemia cancer, renal cancer or prostate cancer.

In yet another aspect, the present disclosure provides methods of treating diseases or disorders characterized by TACC dysregulation in a subject comprising administering a compound disclosed herein or a pharmaceutically acceptable salt thereof to the subject.

In certain embodiments, the TACC is TACC1. In other embodiments, the TACC is TACC2. In other preferred embodiments, the TACC is TACC3.

In certain embodiments, the disease or disorder is cancer. In certain embodiments, the cancer is breast cancer, colon cancer, melanoma cancer, lung cancer, central nervous system cancer, ovarian cancer, leukemia cancer, renal cancer or prostate cancer.

In yet another aspect, the present disclosure provides methods of treating cancer in a subject comprising administering a compound disclosed herein or a pharmaceutically acceptable salt thereof to the subject. In certain embodiments, the cancer is breast cancer, colon cancer, melanoma cancer, lung cancer, central nervous system cancer, ovarian cancer, leukemia cancer, renal cancer or prostate cancer.

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a selfemulsifying drug delivery system or a selfmicroemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit AR or promote AR degradation may render them suitable as “therapeutic agents” in the methods and compositions of this disclosure.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. Coalkyl indicates a hydrogen where the group is in a termil position, a bond if interl. A C₁₋₆alkyl group, for example, contains from one to six carbon atoms in the chain. The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

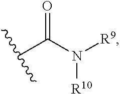

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

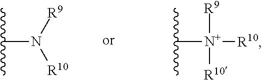

wherein R⁹, R¹⁰, and R¹⁰, each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

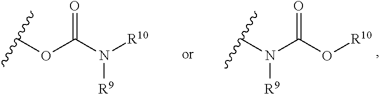

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “cycloalkyl” includes substituted or unsubstituted non-aromatic single ring structures, preferably 4- to 8-membered rings, more preferably 4- to 6-membered rings. The term “cycloalkyl” also includes polycyclic ring systems having two or more cyclic rings in which two or more carbons are common to two adjoining rings wherein at least one of the rings is cycloalkyl and the substituent (e.g., R¹⁰⁰) is attached to the cycloalkyl ring, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, pyrimidine, denzodioxane, tetrahydroquinoline, and the like. The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

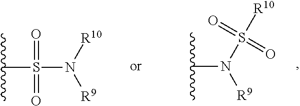

wherein R⁹ and R¹⁰ independently represents hydrogen or hydrocarbyl.

The term “sulfoxide” is art-recognized and refers to the group —S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR9 or —SC(O)R9
wherein R⁹ represents a hydrocarbyl.

The term “thioether”, as used herein, is equivalent to an ether, wherein the oxygen is replaced with a sulfur.

The term “urea” is art-recognized and may be represented by the general formula

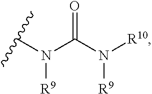

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinmic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as entiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “Log S” or “log S” as used herein is used in the art to quantify the aqueous solubility of a compound. The aqueous solubility of a compound significantly affects its absorption and distribution characteristics. A low solubility often goes along with a poor absorption. Log S value is a unit stripped logarithm (base 10) of the solubility measured in mol/liter.

### EXAMPLES

The invention now being generally described, it will be more readily understood by reference to the following examples which are included merely for purposes of illustration of certain aspects and embodiments of the present invention and are not intended to limit the invention.

### Example 1: Synthesis of Exemplary Compounds of the Disclosure

### General Procedure for the Preparation of Compounds 5-41

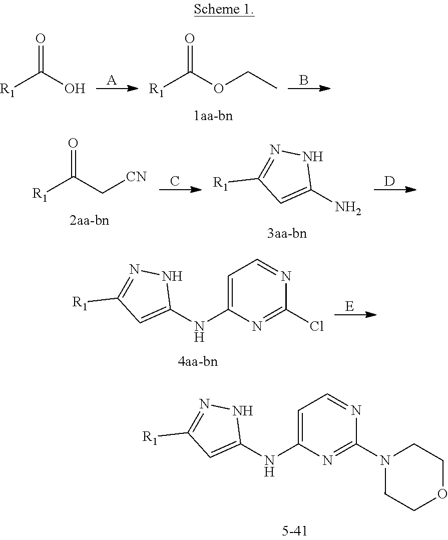

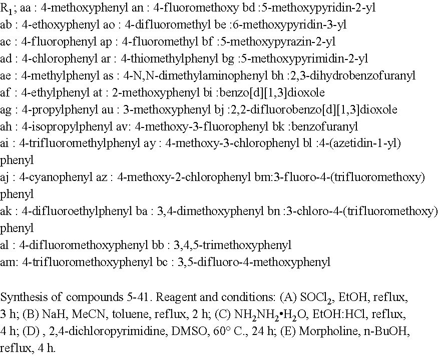

The synthetic methodology for compounds 5-41 is shown in Scheme 1, comprising the following process steps:
i) The appropriate acid derivative was esterified by refluxing in EtOH (ethanol) in the presence of SOCl₂ (thionyl chloride) to produce compounds 1aa-bn,
ii) The solution of compounds 1aa-bn in toluene was refluxed with acetonitrile in the presence of NaH (sodium hydride; 60% dispersion in mineral oil) to obtain the β-keto nitrile derivatives 2aa-bn,
iii) Compounds 2aa-bn were reacted with NH₂NH₂·H₂O (hydrazine monohydrate) in EtOH:conc·HCl (8:1) solution at 95° C. to obtain 1H-pyrazol-5-amine analogues 3aa-bn,
iv) Compounds 3aa-bn was reacted with 2,4-dichloropyrimidine in DMSO (dimethyl sulfoxide) in the presence of DIEA (N,N-diisopropylethylamine) at 60° C. to obtain compounds 4aa-bn,

Compounds 4aa-bn in n-BuOH (butanol) were refluxed with morpholine to produce compounds 5-41. For compound 137 and 138, intermediate 4aj and 4bn in n-BuOH (butanol) were refluxed with 2,6-dimethylmorpholine instead of morpholine to produce final compounds. Preparation of compound 43.

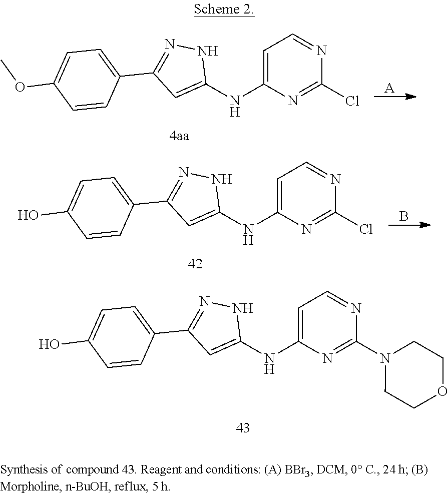

The synthetic methodology for compound 43 is shown in Scheme 2, comprising the following process steps:
i) Demethylation of compound 4aa in DCM (dichloromethane) in the presence of BBr₃ (boron tribromide) at 0° C. was conducted to obtain compound 42,
ii) Compound 42 in n-BuOH was refluxed with morpholine to obtain compound 43.

### General Procedure for Preparation of Compounds 56-58.

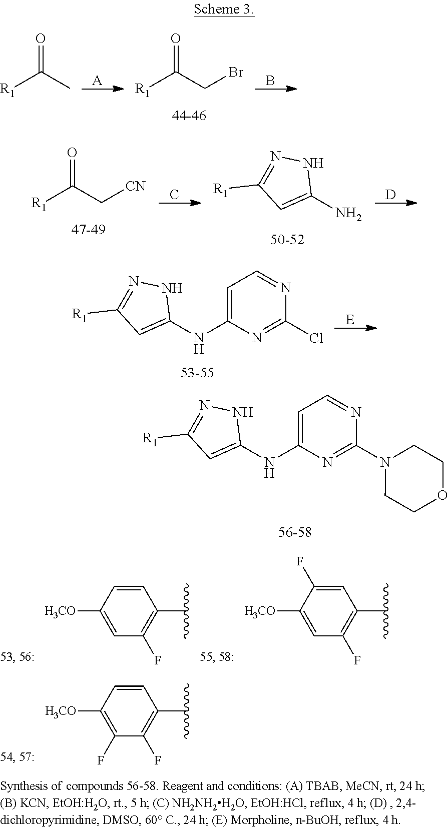

The synthetic methodology for compounds 56-58 is shown in Scheme 1, comprising the following process steps:
i) The solution of appropriate acetophenone derivative in acetonitrile was brominated with TBAB (tetrabutylammonium bromide) to produce compounds 44-46,
ii) The solution of compounds 44-46 (0,2 M) in EtOH:H₂O was reacted with KCN (potassium cyanide) to obtain the β-keto nitrile derivatives 47-49,
iii) Compounds 47-49 were reacted with NH₂NH₂·H₂O in EtOH:conc·HCl (8:1) solution at 95° C. to obtain 1H-pyrazol-5-amine analogues 50-52,
iv) Compounds 50-52 was reacted with 2,4-dichloropyrimidine in DMSO in the presence of DIEA at 60° C. to obtain compounds 53-55,
v) Compounds 53-55 in n-BuOH were refluxed with morpholine to produce compounds 56-58.

### General Procedure for Preparation of Compounds 59-73.

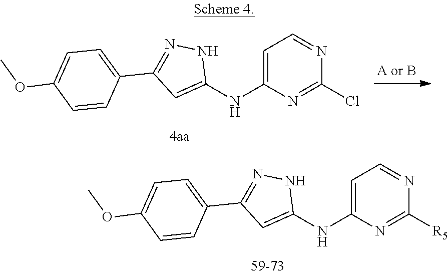

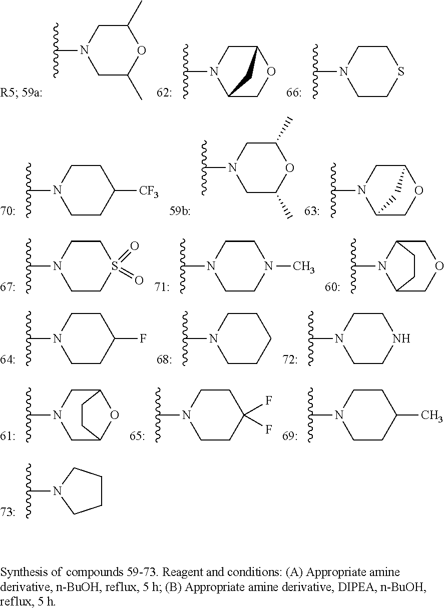

For the synthesis of derivatives with R₅ modifications, synthetic procedures shown in Scheme 4 were utilized. Compound 4aa was used as starting intermediates and then reacted with various amines to obtain the final compounds with Method A or B (59-73). If the amine derivatives were in the form of the HCl salt, Method B was used (for 60-65). The synthetic methodology for compounds 59-73 is shown in Scheme 4, comprising the following process steps:
i) Compound 4aa and the appropriate amine derivative, which is not in HCl (hydrogen chloride) salt form, was refluxed in BuOH to obtain compounds 59, 66-73 (Method A). For compound 72, the N-Boc protected piperazine was used and then the protecting group was hydrolyzed with TFA (Trifluoroacetic acid) in DCM to obtain final compound.
ii) If the amine derivatives are in salt form, appropriate HCl salt of the amine was dissolved in BuOH in the presence of DIPEA to obtain the free amine, which was then reacted with Compound 4aa under reflux to obtain final compounds 60-65 (Method B).

### General Procedure for Preparation of Compounds 77-83

For derivatives with B ring modifications, synthetic procedure outlined in Scheme 5 and 6 were utilized. Compound 3aa was used as the starting material, which underwent a nucleophilic aromatic substitution reaction with various pyrimidine derivatives such as 2,4,5-trichloropyrimidine, 2,4-dichloro-5-fluoropyrimidine, 2,4-dichloro-6-methylpyrimidine (77-79). Also, final compounds 81-83 were obtained by coupling reaction with Compound 3aa and morpholino pyridine or morpholino pyrazine derivatives such as 4-(4-bromopyridin-2-yl)morpholine, 4-(6-bromopyridin-2-yl)morpholine, 4-(6-bromopyrazin-2-yl)morpholine (81-83).

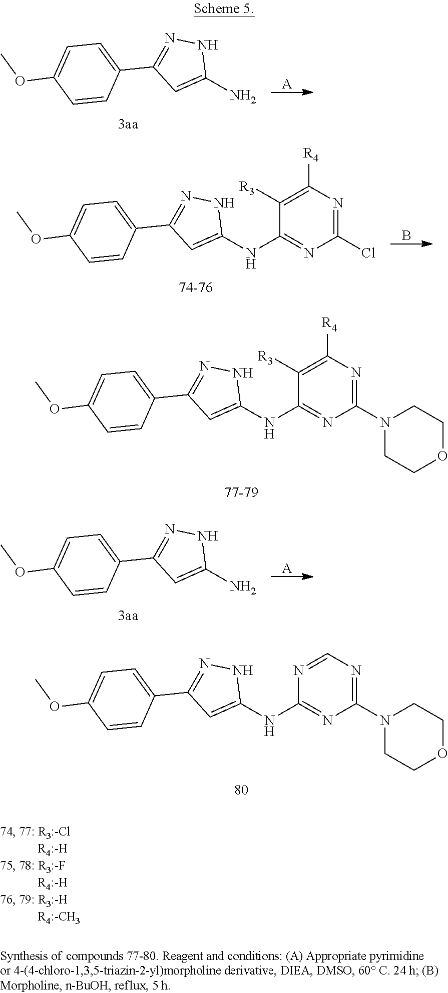

The synthetic methodology for compounds 77-80 is shown in Scheme 5, comprising the following process steps:
i) Compound 3aa was reacted with an appropriate pyrimidine derivative (for Compound 80, 4-(4-chloro-1,3,5-triazin-2-yl)morpholine derivative was used instead of pyrimidine derivative) in DMSO in the presence of DIEA 60° C. to obtain compounds 74-76, 80.
ii) Compounds 74-76 were refluxed with morpholine to produce compounds 77-79.

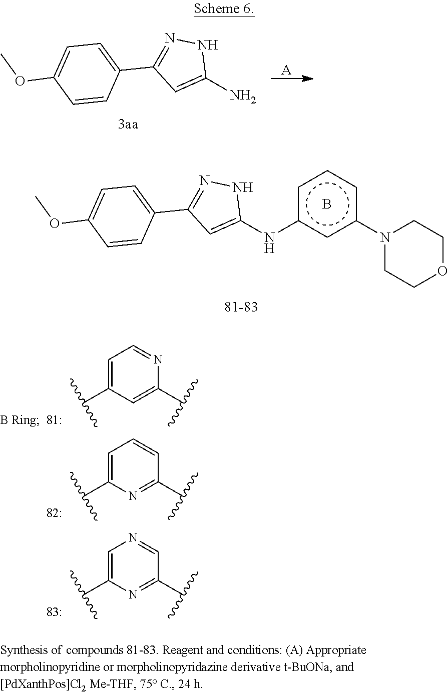

The synthetic methodology for compounds 81-83 is shown in Scheme 6, comprising the following process steps:
i) Compound 3aa was reacted with an appropriate morpholinopyridine or morpholinopyrazine derivative in Me-THF (methyl-tetrahydrofuran) in the presence of t-BuONa (sodium tert-butoxide) and [PdXanthPos]Cl₂ at 75° C. to obtain compounds 81-83.

### General Procedure for Preparation of Compounds 84-85.

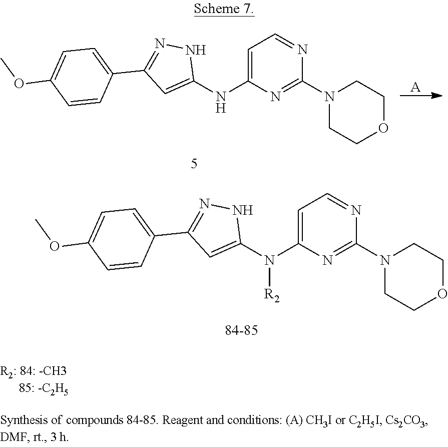

For the synthesis of compounds 84-85, synthetic procedure outlined in Scheme 7 was utilized. Compound 5 was used as the starting material, which was alkylated to obtain the N-methylated and N-ethylated derivatives 84-85.

The synthetic methodology of compounds 84-85 is shown in Scheme 7, comprising the following process step:
i) Compound 5 was dissolved in DMF (N,N-dimethyl formamide) and reacted with CH₃I (iodomethane) or C₂H₅I (iodoethane) in the presence of Cs₂CO₃ (cesium carbonate) at room temperature to obtain the compound 84-85.

### General Procedure for Preparation of Compounds 86-123, 165-212.

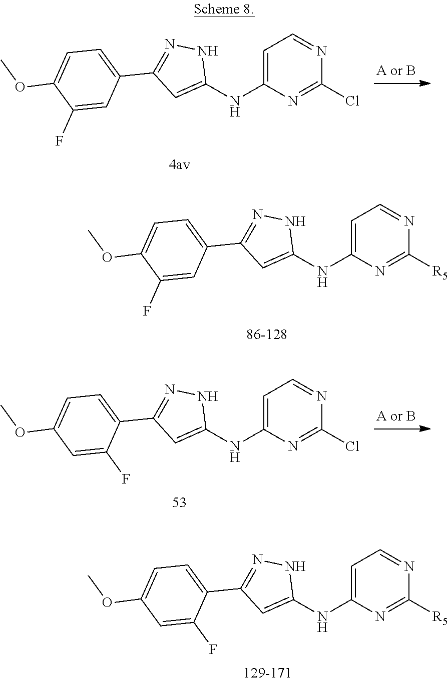

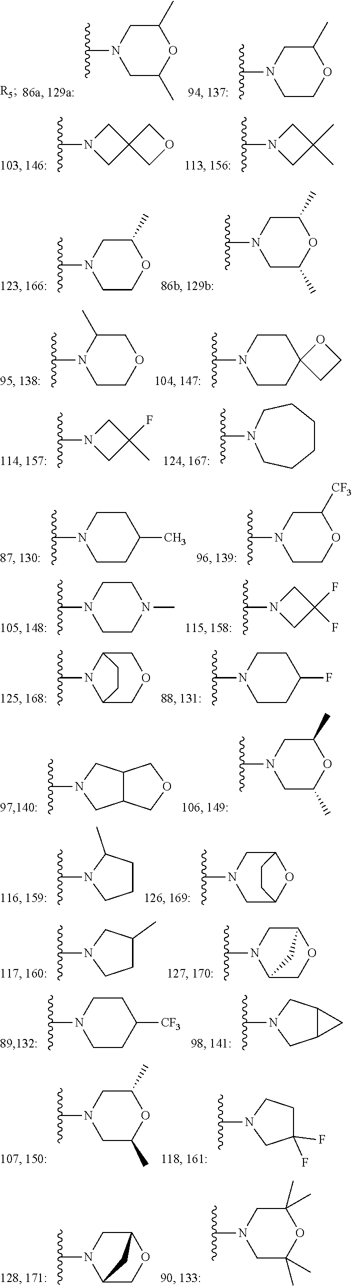

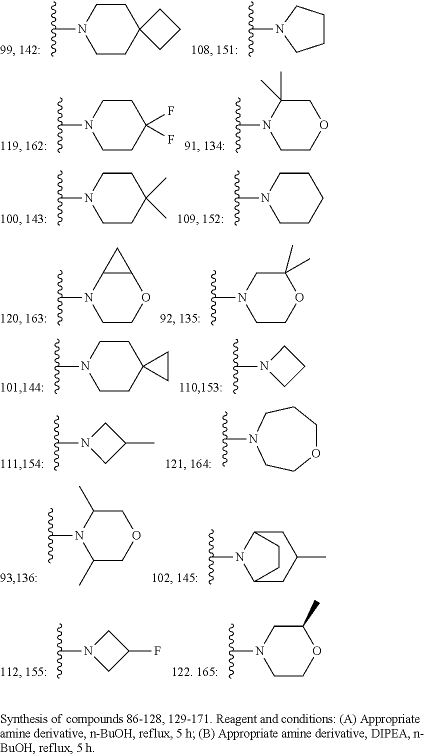

For the synthesis of derivatives with R₅ modifications, synthetic procedures outlined in Scheme 8 were utilized. Hence, compound 4av or compound 53 were used as starting intermediates and then treated with various amine derivatives to afford the final compounds with Method A or B. If the amine derivatives were in the form of the salt form, Method B was used.

The synthetic methodology for compounds 86-171 is shown in Scheme 8, comprising the following process steps:
i) Compound 4av or compound 53 and the appropriate amine derivative, which is not in salt form, was refluxed in BuOH to obtain final compounds (Method A).
ii) If the amine derivatives are in salt form, appropriate salt of the amine was dissolved in BuOH in the presence of DIPEA to obtain the free amine, which was then reacted with Compound 4av or compound 53 reacted with morpholine under reflux to obtain final compounds (Method B).

### Preparation of Compounds 174

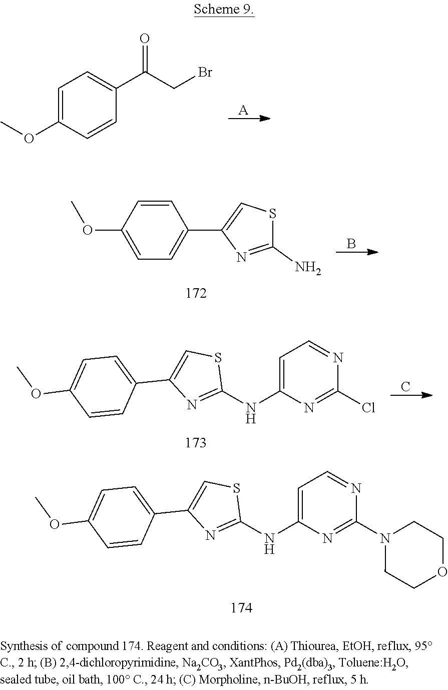

For the synthesis of derivative with thiazole ring modification instead of 1H-pyrazol, synthetic procedures shown in Scheme 9 was utilized. 2-bromo-1-(4-methoxyphenyl)ethan-1-one was used as starting material and then reacted with thiourea to obtain thiazol-2-amine ring. The amine derivative treated with 2,4-dichloropyrimidin by coupling reaction to obtain starting intermediate. Lastly, the intermediates reacted with morpholine under reflux to obtain final compound 174.

The synthetic methodology for compound 174 is shown in Scheme 9, comprising the following process steps:
i) The solution of 2-bromo-1-(4-methoxyphenyl)ethan-1-one in EtOH was reacted with thiourea to produce compound 172,
ii) The solution of compound 172 in Toluene:H₂O (4:1) was degassed under nitrogen atmosphere and then reacted with 2,4-dichloropyrimidine in the presence of Na₂CO₃, XantPhos, Pd₂(dba)₃ to obtain the compound 173,
iii) Compound 173 was treated with morpholine under reflux to afford final compound 174.

### Preparation of Compounds 177

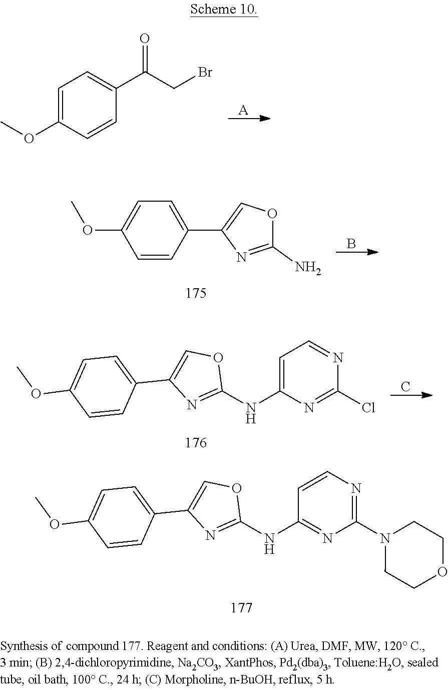

For the synthesis of derivative with oxazole ring modification instead of 1H-pyrazol, synthetic procedures shown in Scheme 10 was utilized. 2-bromo-1-(4-methoxyphenyl)ethan-1-one was used as starting material and then reacted with urea under MW (microwave) condition to afford oxazole-2-amine ring. The amine derivative reacted with 2,4-dichloropyrimidin by coupling reaction to obtain starting intermediates. Lastly, the intermediate reacted with morpholine under reflux to obtain final compound 177.

The synthetic methodology for compound 177 is shown in Scheme 10, comprising the following process steps:
i) The solution of 2-bromo-1-(4-methoxyphenyl)ethan-1-one in DMF was reacted with urea under MW condition to produce compound 175,
ii) The solution of oxazole-2-amine derivative (compound 175) in Toluene:H₂O (4:1) was degassed under nitrogen atmosphere and then reacted with 2,4-dichloropyrimidine in the presence of Na₂CO₃, XantPhos, Pd₂(dba)₃ to obtain the compound 176,
iii) Compound 176 was treated with morpholine under reflux to afford final compound 177.

### Preparation of Compounds 181

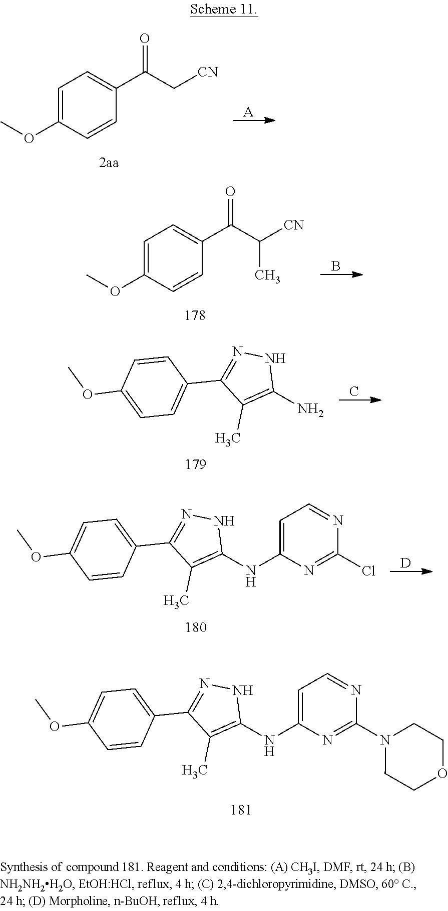

For the synthesis of derivative with 4-methyl-1H-pyrazol ring modification instead of 1H-pyrazol, synthetic procedures shown in Scheme 11 was utilized. Compound 2aa was used as starting material treated with CH₃I to afford methylated β-keto nitrile derivative and then reacted with hydrazine monohydrate to obtain 4-methyl-1H-pyrazol-5-amine derivative (Compound 179). Compound 179 was used as the starting material, which underwent a nucleophilic aromatic substitution reaction with 2,4-dichloropyrimidine to afford intermediate compound 180. Finally, the intermediate reacted with morpholine under reflux to obtain compound 181.

The synthetic methodology for compound 181 is shown in Scheme 11, comprising the following process steps:
i) The solution of compound 2aa in DMF was reacted with CH₃I in the presence of NaH to produce compound 178,
ii) The solution of compound 178 in EtOH:HCl (8:1) treated with NH₂NH₂·H₂O to obtain the amin derivative compound 179,
iii) Compound 179 was dissolved in DMSO and then reacted with 2,4-dichloropyrimidine in the presence of DIEA to afford compound 180,
iv) The solution of compound 180 in n-BuOH reacted with morpholine under reflux to afford final compound 181.

### Preparation of Compounds 184, 187 and 190

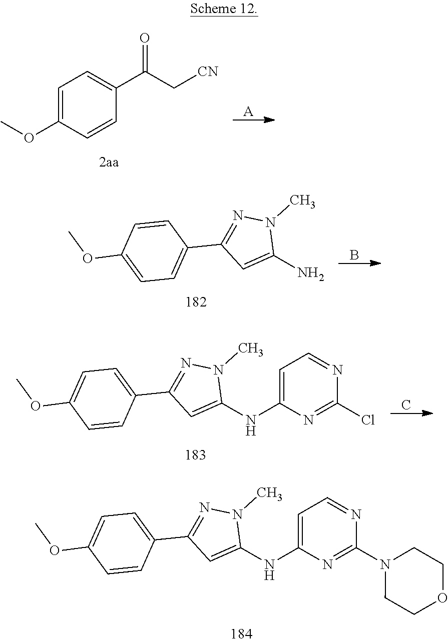

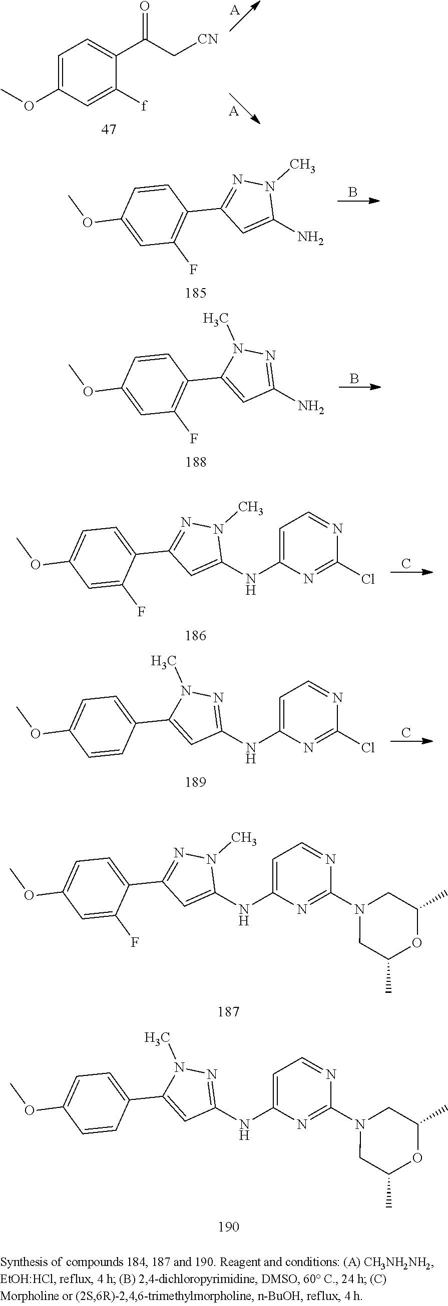

For the synthesis of derivative with 1-methyl-1H-pyrazol ring modification instead of 1H-pyrazol, synthetic procedures shown in Scheme 12 was utilized. Compound 2aa was used as starting material reacted with methylhydrazine to obtain 1-methyl-1H-pyrazol-5-amine derivative (Compound 134). Compound 134 was used as the starting material, which underwent a nucleophilic aromatic substitution reaction with 2,4-dichloropyrimidine to afford intermediate compound 135. Finally, the intermediate reacted with morpholine under reflux to obtain compound 136.

The synthetic methodology for compounds 184, 187 and 190 is shown in Scheme 12, comprising the following process steps:
i) The solution of compound 2aa or 47 in EtOH:HCl (8:1) treated with methylhydrazine to obtain the amin derivative compounds 182, 185, 188 (minor isomer),
ii) Compounds 182, 185, 188 (minor isomer) were dissolved in DMSO and then reacted with 2,4-dichloropyrimidine in the presence of DIEA to afford compound compounds 183, 186, 189,
iii) The solution of compounds 183, 186, 189 in n-BuOH reacted with morpholine or (2S,6R)-2,4,6-trimethylmorpholine under reflux to afford final compounds 184, 187 and 190.

The embodiments of present invention include the chemical structures of the original intermediate compounds, which are reacted with amine derivatives described herein, but not limited to, for the synthesis of compounds of the general Formula (I), and can be selected from the Compounds Listed in Table 1.

### General Procedure for the Preparation of Compounds 191-218.

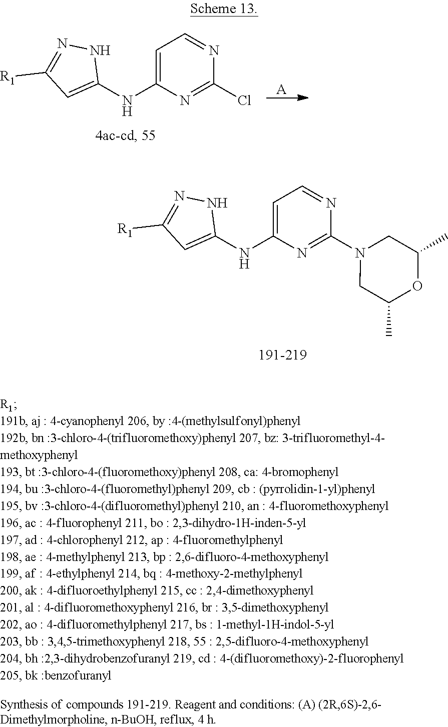

The synthetic methodology for compounds 191-219 is shown in Scheme 13. The compounds 4ac-bs obtained following procedure shown in Scheme 1. The compounds 4ac-cd, 55 in n-BuOH (butanol) were refluxed with (2R,6S)-2,6-dimethylmorpholine to produce compounds 191-219.

### General Procedure for the Preparation of Compounds 220-264 and Preparation of Compound 265.

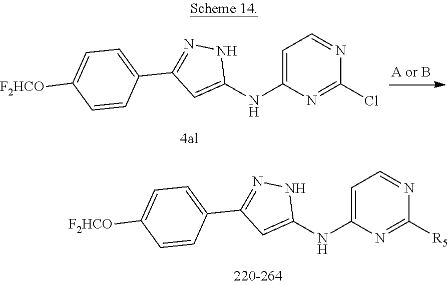

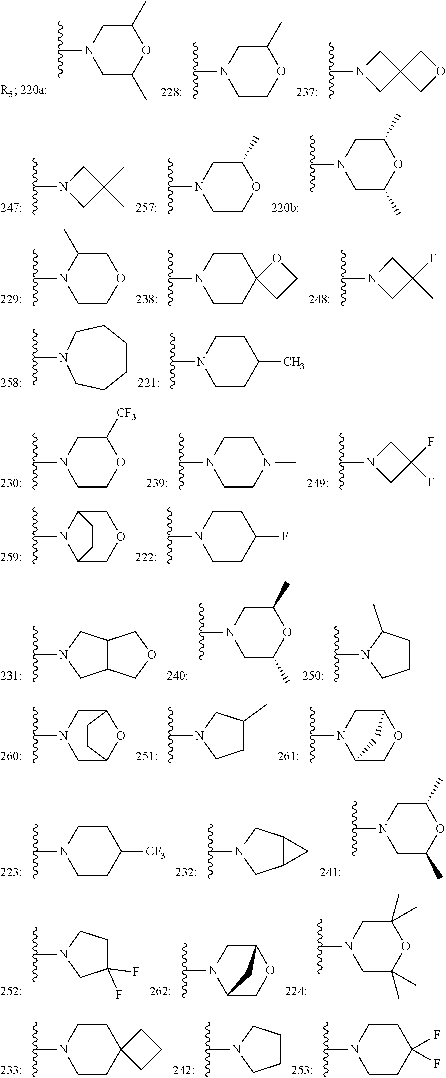

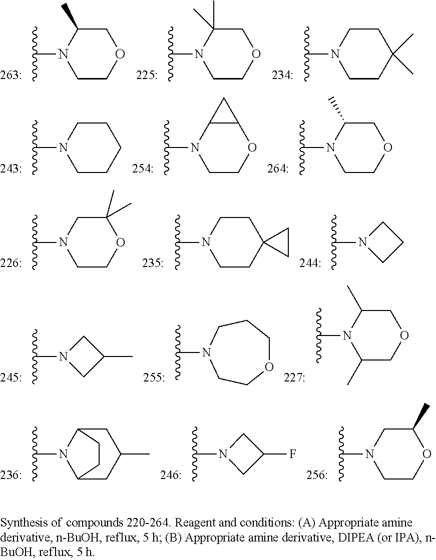

The synthetic methodology for compounds 220-264 is shown in Scheme 14, comprising the following process steps:
iii) Compound 4al and the appropriate amine derivative, which is not in HCl (hydrogen chloride) salt form, was refluxed in BuOH to obtain final compounds (Method A).
iv) If the amine derivatives are in salt form, appropriate HCl salt of the amine was dissolved in BuOH in the presence of DIPEA or IPA to obtain the free amine, which was then reacted with Compound 4al under reflux to obtain final compounds (Method B).

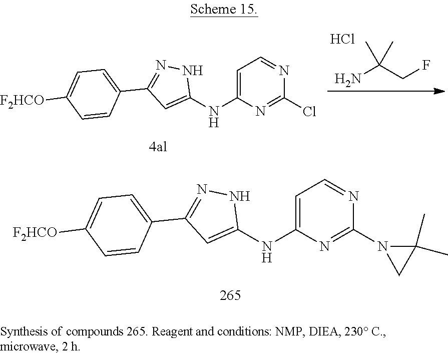

The synthetic methodology for compound 265 is Scheme 15, comprising the following process steps:
i) Compound 4al was reacted with 1-Fluoro-2-methyl-2-propanamine hydrochloride in DMSO in the presence of DIEA, reacted under microwave 230° C. to obtain compounds 265.

### General Procedure for the Preparation of Compounds 274-277

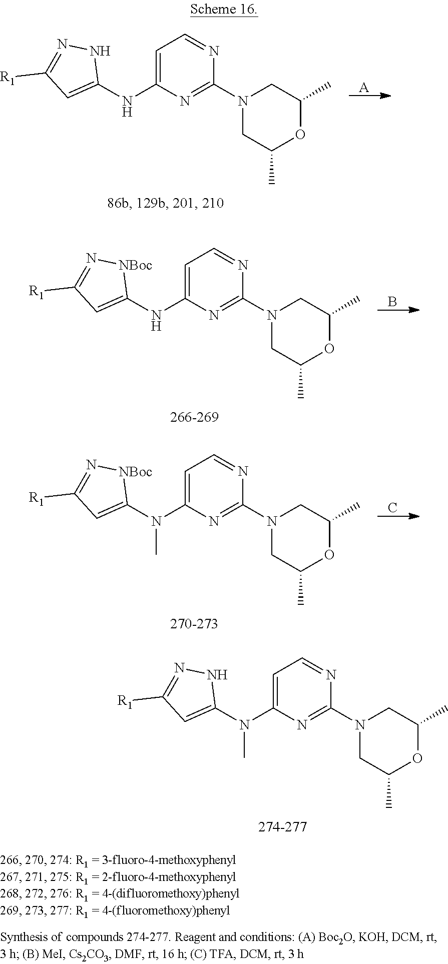

The synthetic methodology for compound 274-277 is Scheme 16, comprising the following process steps:
i) The solution of compound 86b, 129b, 201, or 210 in DCM treated with Boc₂O and KOH to obtain the Boc protected derivatives,
ii) The Boc protected compound was dissolved in DMF and reacted with Cs₂CO₃ and Mel under rt for 16h to afford methylated products,
iii) The obtained compound was treated with TFA in DCM and stirred for 3 h under rt to afford final compounds 274-277.

### General Procedure for the Preparation of Compounds 281-283 and 286-295

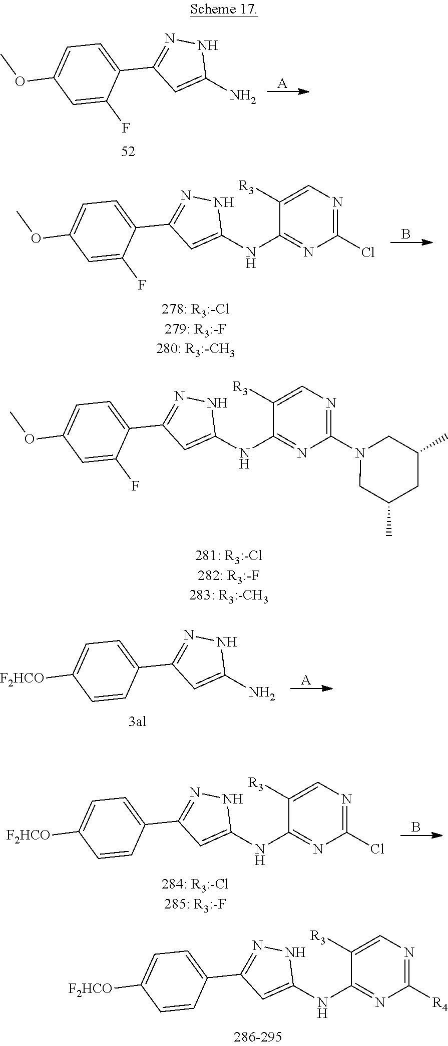

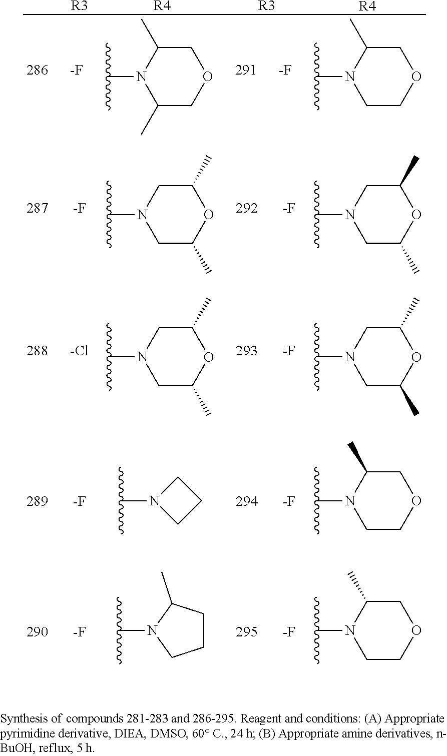

The synthetic methodology for compounds 281-283 and 286-295 is shown in Scheme 17, comprising the following process steps:
i) Compounds 3al or 52 was reacted with an appropriate pyrimidine derivative in DMSO in the presence of DIEA 60° C. to obtain compounds 278-280, 284-285.
ii) Compounds 278-280, 284-285 were refluxed with appropriate amine derivatives to produce compounds 281-283, 286-295.

### General Procedure for the Preparation of Compound 298

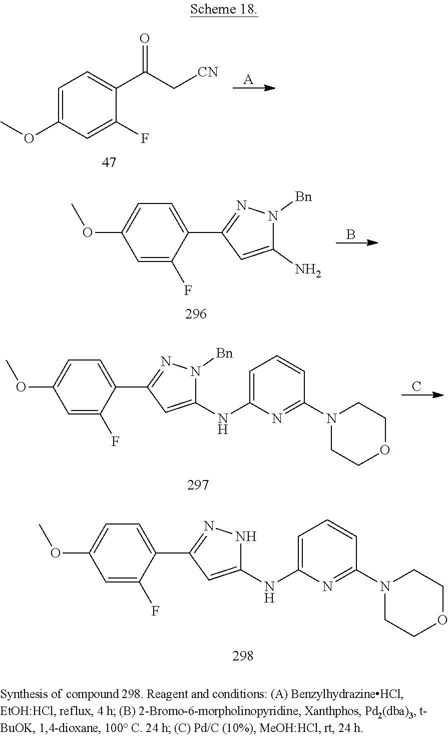

The synthetic methodology for compounds 298 is shown in Scheme 18, comprising the following process steps:
i) Compound 47 was reacted with benzylhydrazine·HCl in EtOH:conc·HCl (8:1) solution at 95° C. to obtain compound 296,
ii) Solution of Compound 296 in dioxane was reacted with 2-bromo-6-morpholinopyridine in the presence of Xanthphos, Pd₂(dba)₃, t-BuOK to produce compounds 297,
iii) Compound 298 was obtained by acid-catalyzed debenzylation of a solution of compound 297 in methanol.

Compounds 299-304 follow the general procedure in Scheme 18.

### General Procedure for the Preparation of Compounds 308-311, 315-316

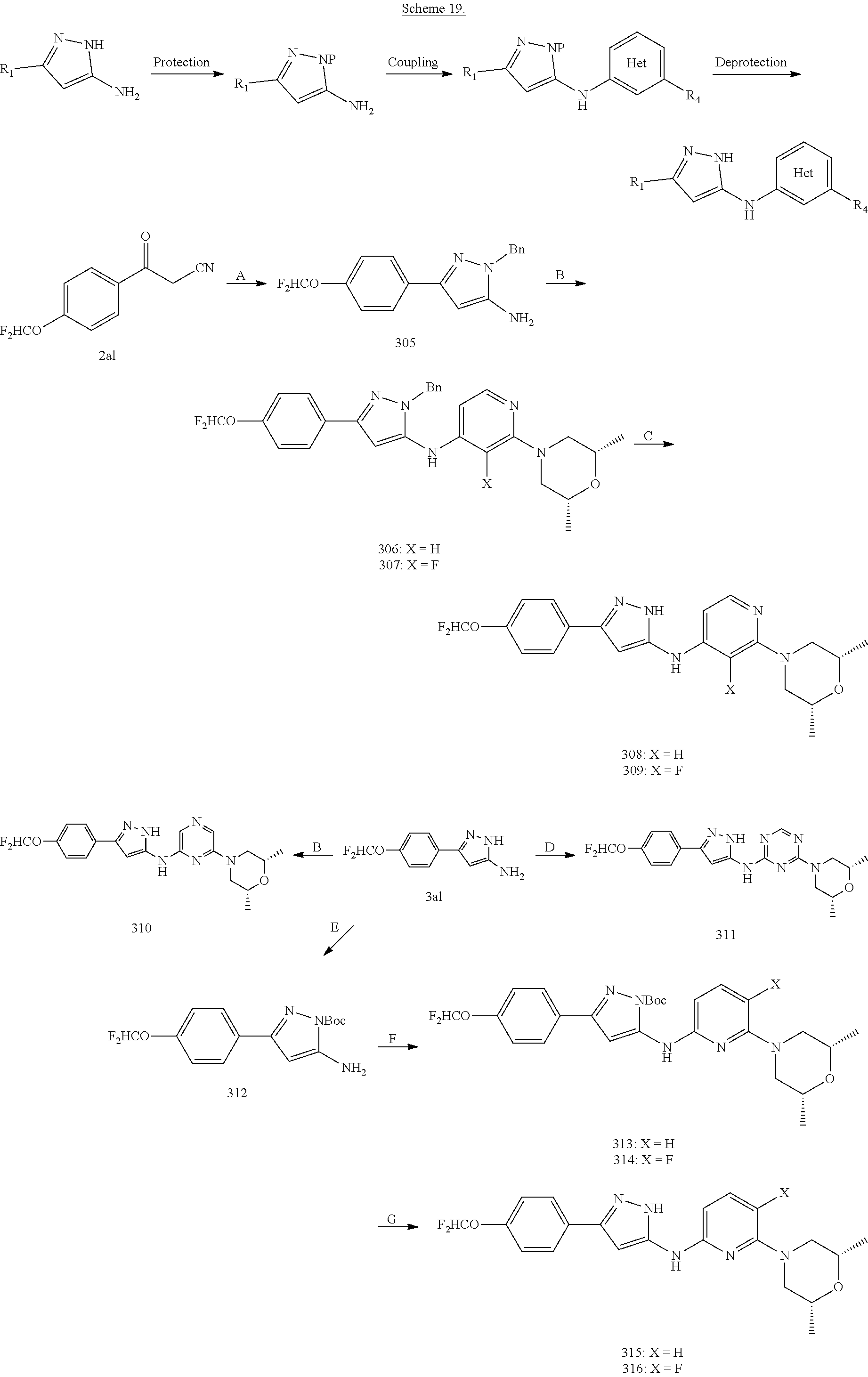

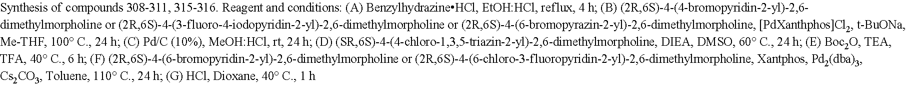

The synthetic methodology for compounds 308-311, 315-316 is shown in Scheme 19, comprising the following process steps:
i) Compound 2al was reacted with benzylhydrazine·HCl in EtOH:conc·HCl (8:1) solution at 95° C. to obtain compound 305,
ii) Solution of Compound 305 in Me-THF was reacted with (2R,6S)-4-(6-bromopyridin-2-yl)-2,6-dimethylmorpholine or (2R,6S)-4-(3-fluoro-4-iodopyridin-2-yl)-2,6-dimethylmorpholine or (2R,6S)-4-(6-bromopyrazin-2-yl)-2,6-dimethylmorpholine in the presence of [PdXanthphos]Cl₂, t-BuONa to produce compounds 306, 307 and 311,
iii) Compound 308 and 309 were obtained by acid-catalyzed debenzylation of a solution of compound 306 and 307 in methanol.
iv) Compound 3al was reacted with (2R,6S)-4-(4-chloro-1,3,5-triazin-2-yl)-2,6-dimethylmorpholine in DMSO in the presence of DIEA 60° C. to obtain compound 310.
v) Compound 3al was reacted with Boc₂O, in the presence of TEA and TFA 40° C. to obtain compound 312.
vi) Compound 312 in toluene was reacted with (2R,6S)-4-(6-bromopyridin-2-yl)-2,6-dimethylmorpholine or (2R,6S)-4-(6-bromo-3-fluoropyridin-2-yl)-2,6-dimethylmorpholine in the presence of Xanthphos, Cs₂CO₃ to produce compounds 313 and 314,
vii) Compounds 313 and 314, deprotected with HCl in dioxane to obtain compounds 315 and 316.

### General Procedure for the Preparation of Compounds 317-318

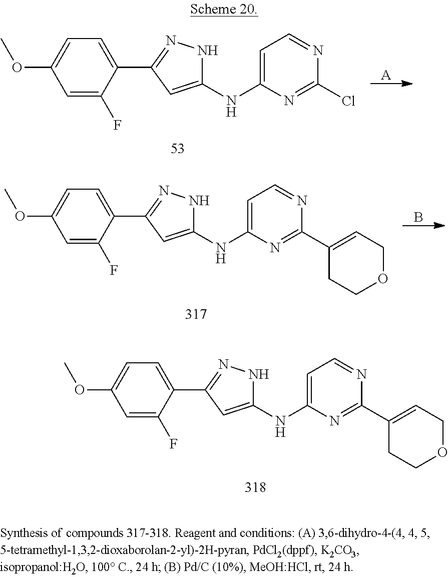

The synthetic methodology for compounds 317-318 is shown in Scheme 20, comprising the following process steps:
i) Compound 53 was reacted with 3,6-dihydro-4-(4, 4, 5, 5-tetramethyl-1,3,2-dioxaborolan-2-yl)-2H-pyran in isopropanol:water (5:1) solution in the presence of PdCl₂(dppf) and K₂CO₃ at 100° C. in sealed tube to obtain compound 317,
ii) Compound 318 was obtained by acid-catalyzed debenzylation of a solution of compound 317.

TABLE 1
Intermediate Compounds of the Disclosure

| Intermediate | | Chemical | Mass |
|---|---|---|---|
| Compound No | | Formula | (found) |
| 4aa | 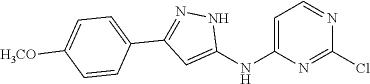 [SMILES: COc1ccc(-c2cc(Nc3ccnc(Cl)n3)[nH]n2)cc1] | C₁₄H₁₂ClN₅O | 302.0716 |
| 4ab | 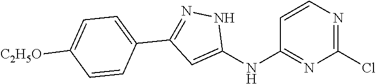 | C₁₅H₁₄ClN₅O | — |
| 4ac | 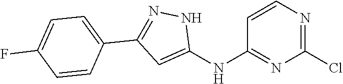 [SMILES: Fc1ccc(-c2cc(Nc3ccnc(Cl)n3)[nH]n2)cc1] | C₁₃H₉ClFN₅ | 289.0531 |
| 4ad | 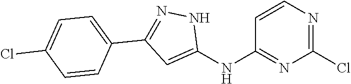 [SMILES: Clc1ccc(-c2cc(Nc3ccnc(Cl)n3)[nH]n2)cc1] | C₁₃H₉Cl₂N₅ | 305.0235 |
| 4ae | 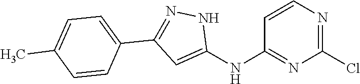 [SMILES: Cc1ccc(-c2cc(Nc3ccnc(Cl)n3)[nH]n2)cc1] | C₁₄H₁₂ClN₅ | 285.0781 |
| 4af | 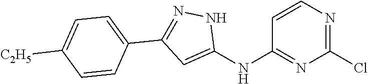 [SMILES: CCc1ccc(-c2cc(Nc3ccnc(Cl)n3)[nH]n2)cc1] | C₁₅H₁₄ClN₅ | 299.0938 |
| 4ag | 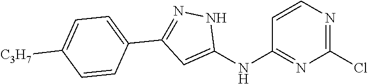 | C₁₆H₁₆ClN₅ | — |
| 4ah | 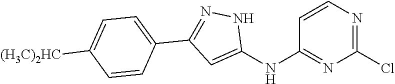 | C₁₆H₁₆ClN₅ | — |
| 4ai | 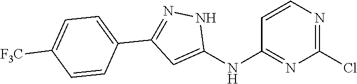 [SMILES: FC(F)(F)c1ccc(-c2cc(Nc3ccnc(Cl)n3)[nH]n2)cc1] | C₁₄H₉ClF₃N₅ | |
| 4aj | 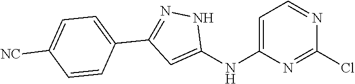 [SMILES: N#Cc1ccc(-c2cc(Nc3ccnc(Cl)n3)[nH]n2)cc1] | C₁₄H₉ClN₆ | 297.0695 |
| 4ak | 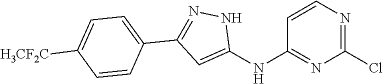 | C₁₅H₁₂ClF₂N₅ | 335.0749 |
| 4al | 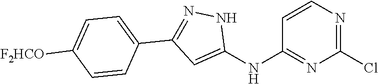 [SMILES: FC(F)Oc1ccc(-c2cc(Nc3ccnc(Cl)n3)[nH]n2)cc1] | C₁₄H₁₀ClF₂N₅O | 337.0542 |
| 4am | 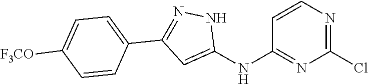 [SMILES: FC(F)(F)Oc1ccc(-c2cc(Nc3ccnc(Cl)n3)[nH]n2)cc1] | C₁₄H₉ClF₃N₅O | — |
| 4an | 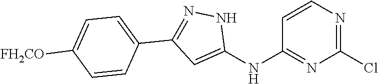 | C₁₄H₁₁ClFN₅O | — |
| 4ao | 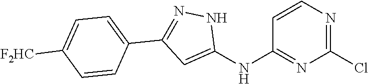 [SMILES: FC(F)c1ccc(-c2cc(Nc3ccnc(Cl)n3)[nH]n2)cc1] | C₁₄H₁₀ClF₂N₅ | 321.0593 |
| 4ap | 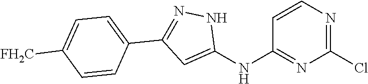 | C₁₄H₁₁ClFN₅ | — |
| 4ar | 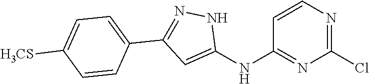 | C₁₄H₁₂ClN₅S | — |
| 4as | 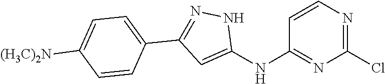 | C₁₅H₁₅ClN₆ | — |
| 4at | 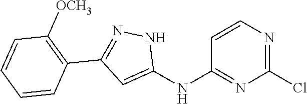 [SMILES: COc1ccccc1-c1cc(Nc2ccnc(Cl)n2)[nH]n1] | C₁₄H₁₂ClN₅O | — |
| 4au | 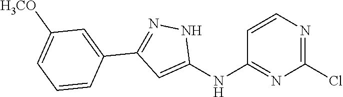 [SMILES: COc1cccc(-c2cc(Nc3ccnc(Cl)n3)[nH]n2)c1] | C₁₄H₁₂ClN₅O | — |
| 4av | 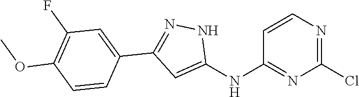 [SMILES: COc1ccc(-c2cc(Nc3ccnc(Cl)n3)[nH]n2)cc1F] | C₁₄H₁₁ClFN₅O | 319.0636 |
| 4ay | 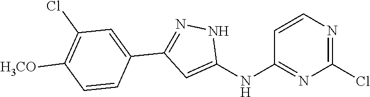 [SMILES: COc1ccc(-c2cc(Nc3ccnc(Cl)n3)[nH]n2)cc1Cl] | C₁₄H₁₁Cl₂N₅O | — |
| 4az | 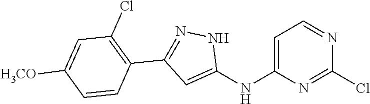 [SMILES: COc1ccc(-c2cc(Nc3ccnc(Cl)n3)[nH]n2)c(Cl)c1] | C₁₄H₁₁Cl₂N₅O | 336.0386 |
| 4ab | 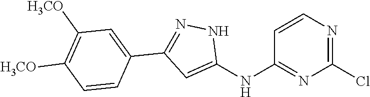 [SMILES: COc1ccc(-c2cc(Nc3ccnc(Cl)n3)[nH]n2)cc1OC] | C₁₅H₁₄ClN₅O₂ | — |
| 4bb | 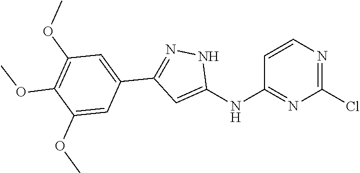 [SMILES: COc1cc(-c2cc(Nc3ccnc(Cl)n3)[nH]n2)cc(OC)c1OC] | C₁₆H₁₆ClN₅O₃ | 361.0942 |
| 4bc | 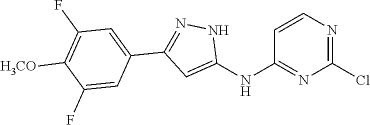 [SMILES: COc1c(F)cc(-c2cc(Nc3ccnc(Cl)n3)[nH]n2)cc1F] | C₁₄H₁₀ClF₂N₅O | — |
| 4bd | 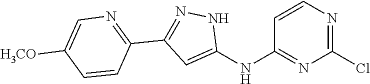 [SMILES: COc1ccc(-c2cc(Nc3ccnc(Cl)n3)[nH]n2)nc1] | C₁₃H₁₁ClN₆O | — |
| 4be | 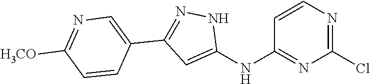 [SMILES: COc1ccc(-c2cc(Nc3ccnc(Cl)n3)[nH]n2)cn1] | C₁₃H₁₁ClN₆O | — |
| 4bf | 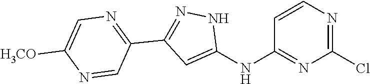 [SMILES: COc1cnc(-c2cc(Nc3ccnc(Cl)n3)[nH]n2)cn1] | C₁₂H₁₀ClN₇O | — |
| 4bg | 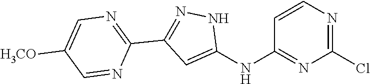 [SMILES: COc1cnc(-c2cc(Nc3ccnc(Cl)n3)[nH]n2)nc1] | C₁₂H₁₀ClN₇O | — |
| 4bh | 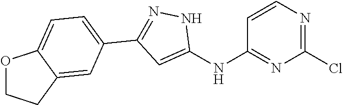 [SMILES: Clc1nccc(Nc2cc(-c3ccc4c(c3)CCO4)n[nH]2)n1] | C₁₅H₁₂ClN₅O | 313.0730 |
| 4bi | 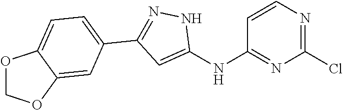 [SMILES: Clc1nccc(Nc2cc(-c3ccc4c(c3)OCO4)n[nH]2)n1] | C₁₄H₁₀ClN₅O₂ | — |
| 4bj | 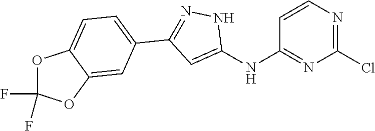 [SMILES: FC1(F)Oc2ccc(-c3cc(Nc4ccnc(Cl)n4)[nH]n3)cc2O1] | C₁₄H₈ClF₂N₅O₂ | — |
| 4bk | 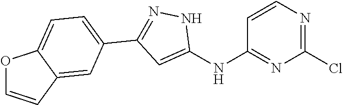 [SMILES: Clc1nccc(Nc2cc(-c3ccc4occc4c3)n[nH]2)n1] | C₁₅H₁₀ClN₅O | 311.0574 |
| 4bl | 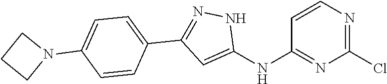 [SMILES: Clc1nccc(Nc2cc(-c3ccc(N4CCC4)cc3)n[nH]2)n1] | C₁₆H₁₅ClN₆ | — |
| 4bm | 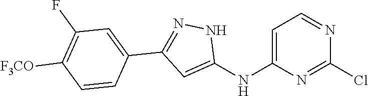 [SMILES: Fc1cc(-c2cc(Nc3ccnc(Cl)n3)[nH]n2)ccc1OC(F)(F)F] | C₁₄H₈ClF₄N₅O | 374.0440 |
| 4bn | 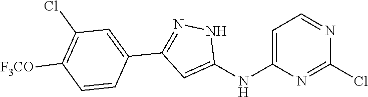 [SMILES: FC(F)(F)Oc1ccc(-c2cc(Nc3ccnc(Cl)n3)[nH]n2)cc1Cl] | C₁₄H₈Cl₂F₃N₅O | 390.0196 |
| 4bo | 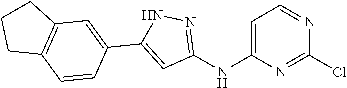 [SMILES: Clc1nccc(Nc2cc(-c3ccc4c(c3)CCC4)[nH]n2)n1] | C₁₆H₁₄ClN₅ | — |
| 4bp | 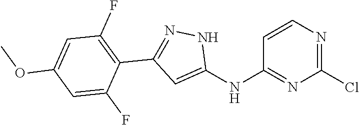 [SMILES: COc1cc(F)c(-c2cc(Nc3ccnc(Cl)n3)[nH]n2)c(F)c1] | C₁₄H₁₀ClF₂N₅O | — |
| 4bq | 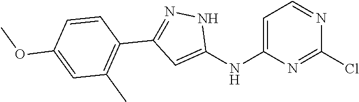 [SMILES: COc1ccc(-c2cc(Nc3ccnc(Cl)n3)[nH]n2)c(C)c1] | C₁₅H₁₄ClN₅O | — |
| 4br | 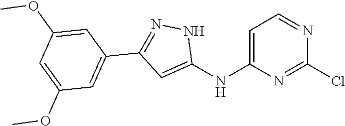 [SMILES: COc1cc(OC)cc(-c2cc(Nc3ccnc(Cl)n3)[nH]n2)c1] | C₁₅H₁₄ClN₅O₂ | — |
| 4bs | 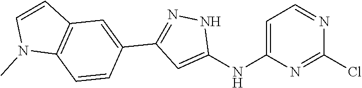 [SMILES: Cn1ccc2cc(-c3cc(Nc4ccnc(Cl)n4)[nH]n3)ccc21] | C₁₆H₁₃ClN₆ | — |
| 4bt | 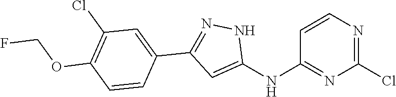 [SMILES: FCOc1ccc(-c2cc(Nc3ccnc(Cl)n3)[nH]n2)cc1Cl] | C₁₄H₁₀Cl₂FN₅O | — |
| 4bu | 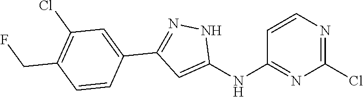 [SMILES: FCc1ccc(-c2cc(Nc3ccnc(Cl)n3)[nH]n2)cc1Cl] | C₁₄H₁₀Cl₂FN₅ | — |
| 4bv | 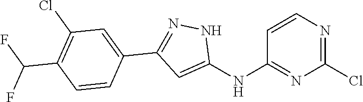 [SMILES: FC(F)c1ccc(-c2cc(Nc3ccnc(Cl)n3)[nH]n2)cc1Cl] | C₁₄H₉Cl₂F₂N₅ | — |
| 4by | 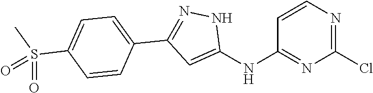 [SMILES: CS(=O)(=O)c1ccc(-c2cc(Nc3ccnc(Cl)n3)[nH]n2)cc1] | C₁₄H₁₂ClN₅O₂S | 349.0400 |
| 4bz | 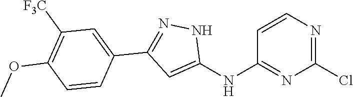 [SMILES: COc1ccc(-c2cc(Nc3ccnc(Cl)n3)[nH]n2)cc1C(F)(F)F] | C₁₅H₁₁ClF₃N₅O | 369.0604 |
| 4ca | 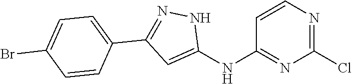 [SMILES: Clc1nccc(Nc2cc(-c3ccc(Br)cc3)n[nH]2)n1] | C₁₃H₉BrClN₅ | 348.9730 |
| 4cb | 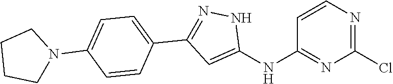 [SMILES: Clc1nccc(Nc2cc(-c3ccc(N4CCCC4)cc3)n[nH]2)n1] | C₁₇H₁₇ClN₆ | 340.1203 |
| 4cc | 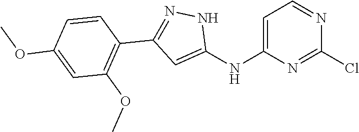 [SMILES: COc1ccc(-c2cc(Nc3ccnc(Cl)n3)[nH]n2)c(OC)c1] | C₁₅H₁₄ClN₅O₂ | — |
| 4cd | 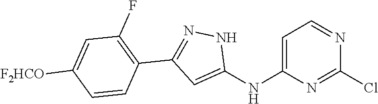 [SMILES: Fc1cc(OC(F)F)ccc1-c1cc(Nc2ccnc(Cl)n2)[nH]n1] | C₁₄H₉ClF₃N₅O | LCMS (ESI⁺): m/z 356.0 [M + 1]⁺, |
| 42 | 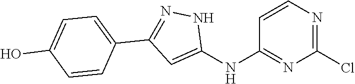 [SMILES: Oc1ccc(-c2cc(Nc3ccnc(Cl)n3)[nH]n2)cc1] | C₁₃H₁₀ClN₅O | — |
| 53 | 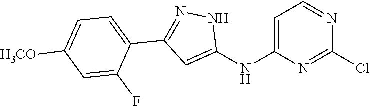 [SMILES: COc1ccc(-c2cc(Nc3ccnc(Cl)n3)[nH]n2)c(F)c1] | C₁₄H₁₁ClFN₅O | 320.0660 |
| 54 | 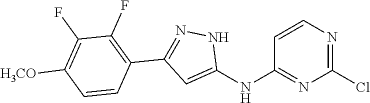 [SMILES: COc1ccc(-c2cc(Nc3ccnc(Cl)n3)[nH]n2)c(F)c1F] | C₁₄H₁₀ClF₂N₅O | — |
| 55 | 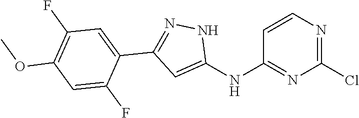 [SMILES: COc1cc(F)c(-c2cc(Nc3ccnc(Cl)n3)[nH]n2)cc1F] | C₁₄H₁₀ClF₂N₅O | 337.0542 |
| 74 | 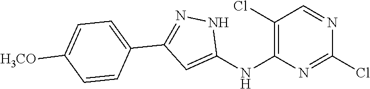 [SMILES: COc1ccc(-c2cc(Nc3nc(Cl)ncc3Cl)[nH]n2)cc1] | C₁₄H₁₁Cl₂N₅O | — |
| 75 | 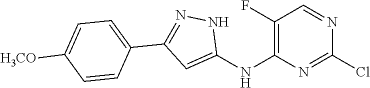 [SMILES: COc1ccc(-c2cc(Nc3nc(Cl)ncc3F)[nH]n2)cc1] | C₁₄H₁₁ClFN₅O | — |
| 76 | 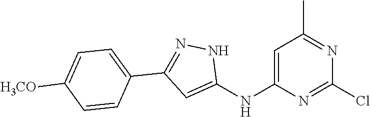 [SMILES: COc1ccc(-c2cc(Nc3cc(C)nc(Cl)n3)[nH]n2)cc1] | C₁₅H₁₄ClN₅O | — |
| 173 | 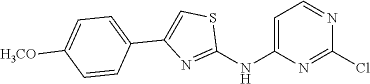 [SMILES: COc1ccc(-c2csc(Nc3ccnc(Cl)n3)n2)cc1] | C₁₄H₁₁ClN₄OS | 319.0338 |
| 176 | 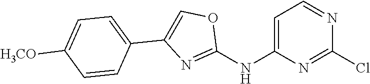 [SMILES: COc1ccc(-c2coc(Nc3ccnc(Cl)n3)n2)cc1] | C₁₄H₁₁ClN₄O₂ | 303.0665 |
| 180 | 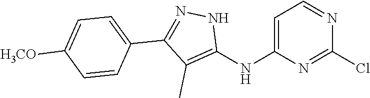 [SMILES: COc1ccc(-c2n[nH]c(Nc3ccnc(Cl)n3)c2C)cc1] | C₁₅H₁₄ClN₅O | 316.0977 |
| 183 | 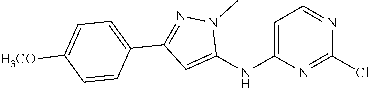 [SMILES: COc1ccc(-c2cc(Nc3ccnc(Cl)n3)n(C)n2)cc1] | C₁₅H₁₄ClN₅O | 316.0906 |
| 278 | 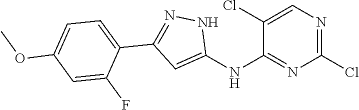 [SMILES: COc1ccc(-c2cc(Nc3nc(Cl)ncc3Cl)[nH]n2)c(F)c1] | C₁₄H₁₀Cl₂FN₅O | 353.0246 |
| 279 | 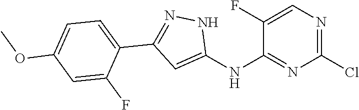 [SMILES: COc1ccc(-c2cc(Nc3nc(Cl)ncc3F)[nH]n2)c(F)c1] | C₁₄H₁₀ClF₂N₅O | 337.0542 |
| 280 | 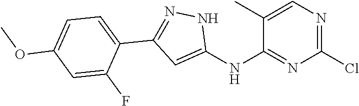 [SMILES: COc1ccc(-c2cc(Nc3nc(Cl)ncc3C)[nH]n2)c(F)c1] | C₁₅H₁₃ClFN₅O | 333.0793 |
| 297 | 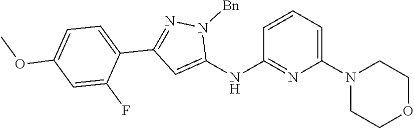 [SMILES: COc1ccc(-c2cc(Nc3cccc(N4CCOCC4)n3)n(Cc3ccccc3)n2)c(F)c1] | C₂₆H₂₆FN₅O₂ | 459.2071 |
| 306 | 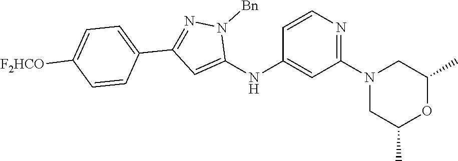 [SMILES: C[C@@H]1CN(c2cc(Nc3cc(-c4ccc(OC(F)F)cc4)nn3Cc3ccccc3)ccn2)C[C@H](C)O1] | C₂₈H2₉F₂N₅O₂ | 505.2289 |

The present disclosure includes compounds, but is not limited to, the compounds disclosed in Table 2

TABLE 2
Summary of Compounds of the Disclosure

| Compound No. | Name | Mass & ¹H/¹³C NMR |
|---|---|---|
| 5 | N-(3-(4-methoxyphenyl)-1H- | [M + H]⁺ 353.1719/¹H NMR (400 MHz, |
| | pyrazol-5-yl)-2- | DMSO-d6): δ 3.67 (8H, bs); 3.79 (3H, s); |
| | morpholinopyrimidin-4-amine | 6.38 (1H, s); 6.68 (1H, bs); 7.02 (2H, d, J = |
| | | 8.0 Hz); 7.63 (2H, d, J = 8.0 Hz); 7.94 (1H, |
| | | d, J = 5.6 Hz); 9.57 (1H, s); 12.60 (1H, |
| | | s). ¹³C NMR (100 MHz, DMSO-d6): δ |
| | | 44.10; 55.21; 66.09; 92.85; 96.52; 114.45; |
| | | 122.14; 126.37; 141.81; 149.08; 156.16; |
| | | 159.13; 159.79; 161.42. |
| 6 | N-(3-(4-ethoxyphenyl)-1H- | — |
| | pyrazol-5-yl)-2- | |
| | morpholinopyrimidin-4-amine | |
| 7 | N-(3-(4-fluorophenyl)-1H- | — |
| | pyrazol-5-yl)-2- | |
| | morpholinopyrimidin-4-amine | |
| 8 | N-(3-(4-chlorophenyl)-1H- | — |
| | pyrazol-5-yl)-2- | |
| | morpholinopyrimidin-4-amine | |
| 9 | 2-morpholino-N-(3-(p-tolyl)- | — |
| | 1H-pyrazol-5-yl)pyrimidin-4- | |
| | amine | |
| 10 | N-(3-(4-ethylphenyl)-1H- | — |
| | pyrazol-5-yl)-2- | |
| | morpholinopyrimidin-4-amine | |
| 11 | 2-morpholino-N-(3-(4- | — |
| | propylphenyl)-1H-pyrazol-5- | |
| | yl)pyrimidin-4-amine | |
| 12 | N-(3-(4-isopropylphenyl)-1H- | — |
| | pyrazol-5-yl)-2- | |
| | morpholinopyrimidin-4-amine | |
| 13 | 2-morpholino-N-(3-(4- | — |
| | (trifluoromethyl)phenyl)-1H- | |
| | pyrazol-5-yl)pyrimidin-4-amine | — |
| 14 | 4-(5-((2-morpholinopyrimidin- | |
| | 4-yl)amino)-1H-pyrazol-3- | |
| | yl)benzonitrile | |
| 15 | N-(3-(4-(1,1- | — |
| | difluoroethyl)phenyl)-1H- | |
| | pyrazol-5-yl)-2- | |
| | morpholinopyrimidin-4-amine | |
| 16 | N-(3-(4- | — |
| | (difluoromethoxy)phenyl)-1H- | |
| | pyrazol-5-yl)-2- | |
| | morpholinopyrimidin-4-amine | |
| 17 | 2-morpholino-N-(3-(4- | — |
| | (trifluoromethoxy)phenyl)-1H- | |
| | pyrazol-5-yl)pyrimidin-4-amine | |
| 18 | N-(3-(4- | — |
| | (fluoromethoxy)phenyl)-1H- | |
| | pyrazol-5-yl)-2- | |
| | morpholinopyrimidin-4-amine | |
| 19 | N-(3-(4- | — |
| | (difluoromethyl)phenyl)-1H- | |
| | pyrazol-5-yl)-2- | |
| | morpholinopyrimidin-4-amine | |
| 20 | N-(3-(4-(fluoromethyl)phenyl)- | — |
| | 1H-pyrazol-5-yl)-2- | |
| | morpholinopyrimidin-4-amine | |
| 21 | N-(3-(4-(methylthio)phenyl)- | — |
| | 1H-pyrazol-5-yl)-2- | |
| | morpholinopyrimidin-4-amine | |
| 22 | N-(3-(4- | — |
| | (dimethylamino)phenyl)-1H- | |
| | pyrazol-5-yl)-2- | |
| | morpholinopyrimidin-4-amine | |
| 23 | N-(3-(2-methoxyphenyl)-1H- | — |
| | pyrazol-5-yl)-2- | |
| | morpholinopyrimidin-4-amine | |
| 24 | N-(3-(3-methoxyphenyl)-1H- | — |
| | pyrazol-5-yl)-2- | |
| | morpholinopyrimidin-4-amine | |
| 25 | N-(3-(3-fluoro-4- | — |
| | methoxyphenyl)-1H-pyrazol-5- | |
| | yl)-2-morpholinopyrimidin-4- | |
| | amine | |
| 26 | N-(3-(3-chloro-4- | — |
| | methoxyphenyl)-1H-pyrazol-5- | |
| | yl)-2-morpholinopyrimidin-4- | |
| | amine | |
| 27 | N-(3-(2-chloro-4- | [M + H]⁺ 387.1309/¹H NMR (400 MHz, |
| | methoxyphenyl)-1H-pyrazol-5- | DMSO-d6): δ 3.64-3.68 (8H, m); 3.83 (3H, |
| | yl)-2-morpholinopyrimidin-4- | s); 6.30 (1H, bs); 6.95 (1H, bs); 7.06 (1H, |
| | amine | dd, J = 8.7; 2.3 Hz); 7.18 (1H, d, J = 2.3 |
| | | Hz); 7.59 (1H, d, J = 8.7 Hz); 7.93 (1H, d, |
| | | J = 5.6 Hz); 9.67 (1H, s); 12.57 (1H, s). ¹³C |
| | | NMR (100 MHz, DMSO-d6): δ 44.56; |
| | | 56.24; 66.49; 97.12; 97.70; 114.35; 116.10; |
| | | 121.43; 131.04; 131.95; 138.82; 149.01; |
| | | 156.43; 160.09; 160.16; 161.86. |
| 28 | N-(3-(3,4-dimethoxyphenyl)- | — |
| | 1H-pyrazol-5-yl)-2- | |
| | morpholinopyrimidin-4-amine | |
| 29 | 2-morpholino-N-(3-(3,4,5- | — |
| | trimethoxyphenyl)-1H-pyrazol- | |
| | 5-yl)pyrimidin-4-amine | |
| 30 | N-(3-(3,5-difluoro-4- | — |
| | methoxyphenyl)-1H-pyrazol-5- | |
| | yl)-2-morpholinopyrimidin-4- | |
| | amine | |
| 31 | N-(3-(5-methoxypyridin-2-yl)- | — |
| | 1H-pyrazol-5-yl)-2- | |
| | morpholinopyrimidin-4-amine | |
| 32 | N-(3-(6-methoxypyridin-3-yl)- | — |
| | 1H-pyrazol-5-yl)-2- | |
| | morpholinopyrimidin-4-amine | |
| 33 | N-(3-(5-methoxypyrazin-2-yl)- | — |
| | 1H-pyrazol-5-yl)-2- | |
| | morpholinopyrimidin-4-amine | |
| 34 | N-(3-(5-methoxypyrimidin-2- | — |
| | yl)-1H-pyrazol-5-yl)-2- | |
| | morpholinopyrimidin-4-amine | |
| 35 | N-(3-(2,3-dihydrobenzofuran-5- | — |
| | yl)-1H-pyrazol-5-yl)-2- | |
| | morpholinopyrimidin-4-amine | |
| 36 | N-(3-(benzo[d][1,3]dioxol-5- | — |
| | yl)-1H-pyrazol-5-yl)-2- | |
| | morpholinopyrimidin-4-amine | |
| 37 | N-(3-(2,2- | — |
| | difluorobenzo[d][1,3]dioxol-5- | |
| | yl)-1H-pyrazol-5-yl)-2- | |
| | morpholinopyrimidin-4-amine | |
| 38 | N-(3-(benzofuran-5-yl)-1H- | — |
| | pyrazol-5-yl)-2- | |
| | morpholinopyrimidin-4-amine | |
| 39 | N-(3-(4-(azetidin-1-yl)phenyl)- | — |
| | 1H-pyrazol-5-yl)-2- | |
| | morpholinopyrimidin-4-amine | |
| 40 | N-(3-(3-fluoro-4- | — |
| | (trifluoromethoxy)phenyl)-1H- | |
| | pyrazol-5-yl)-2- | |
| | morpholinopyrimidin-4-amine | |
| 41 | N-(3-(3-chloro-4- | — |
| | (trifluoromethoxy)phenyl)-1H- | |
| | pyrazol-5-yl)-2- | |
| | morpholinopyrimidin-4-amine | |
| 43 | 4-(5-((2-morpholinopyrimidin- | — |
| | 4-yl)amino)-1H-pyrazol-3- | |
| | yl)phenol | |
| 56 | N-(3-(2-fluoro-4- | [M + H]⁺ 371.1526/¹H-NMR (400 MHz, |
| | methoxyphenyl)-1H-pyrazol-5- | DMSO-d6): δ 3.65-3.66 (8H, m); 3.81 (3H, |
| | yl)-2-morpholinopyrimidin-4- | s); 6.32 (1H, s); 6.84-6.93 (3H, m); 7.11 |
| | amine | (1H, t, J = 8.8 Hz); 7.93 (1H, d, J = 5.2 Hz); |
| | | 9.65 (1H, s); 12.62 (1H, s). ¹³C-NMR (100 |
| | | MHz, DMSO-d6): δ 44.56; 56.25; 66.50; |
| | | 96.30; 97.09; 102.78 (d, ²J_{C-F} = 25.0 Hz); |
| | | 110.32 (d, ²J_{C-F} = 14.0 Hz); 111.59; 128.28; |
| | | 136.14; 149.57; 156.50; 159.58 (d, ¹J_{C-F} = |
| | | 233.0 Hz); 160.11; 160.87; 161.84. |
| 57 | N-(3-(2,3-difluoro-4- | — |
| | methoxyphenyl)-1H-pyrazol-5- | |
| | yl)-2-morpholinopyrimidin-4- | |
| | amine | |
| 58 | N-(3-(2,5-difluoro-4- | — |
| | methoxyphenyl)-1H-pyrazol-5- | |
| | yl)-2-morpholinopyrimidin-4- | |
| | amine | |
| 59a | 2-(2,6-dimethylmorpholino)-N- | — |
| | (3-(4-methoxyphenyl)-1H- | |
| | pyrazol-5-yl)pyrimidin-4-amine | |
| 59b | 2-((2R,6S)-2,6- | [M + H]⁺ 381.1860/¹H-NMR (400 MHz, |
| | dimethylmorpholino)-N-(3-(4- | DMSO-d6): δ 1.15 (6H, d, J = 6.4 Hz); |
| | methoxyphenyl)-1H-pyrazol-5- | 2.46-2.48 (2H, m); 3.54-3.58 (2H, m); 3.78 |
| | yl)pyrimidin-4-amine | (3H, s); 4.49 (2H, m); 6.33 (1H, s); 6.72 |
| | | (1H, bs); 7.01 (2H, d, J = 8.8 Hz); 7.61 (2H, |
| | | d, J = 8.8 Hz); 7.91 (1H, d, J = 5.6 Hz); 9.58 |
| | | (1H, s); 12.59 (1H, s). ¹³C-NMR (100 |
| | | MHz, DMSO-d6): δ 19.26; 49.67; 55.70; |
| | | 71.46; 93.46; 96.81; 114.87; 112.60; |
| | | 126.66; 142.16; 149.55; 156.62; 159.61; |
| | | 160.17; 161.47. |
| 60 | 2-(3-oxa-8- | — |
| | azabicyclo[3.2.1]octan-8-yl)-N- | |
| | (3-(4-methoxyphenyl)-1H- | |
| | pyrazol-5-yl)pyrimidin-4-amine | |
| 61 | 2-(8-oxa-3- | — |
| | azabicyclo[3.2.1]octan-3-yl)-N- | |
| | (3-(4-methoxyphenyl)-1H- | |
| | pyrazol-5-yl)pyrimidin-4-amine | |
| 62 | 2-((1R,4R)-2-oxa-5- | — |
| | azabicyclo[2.2.1]heptan-5-yl)- | |
| | N-(3-(4-methoxyphenyl)-1H- | |
| | pyrazol-5-yl)pyrimidin-4-amine | |
| 63 | 2-((1S,4S)-2-oxa-5- | — |
| | azabicyclo[2.2.1]heptan-5-yl)- | |
| | N-(3-(4-methoxyphenyl)-1H- | |
| | pyrazol-5-yl)pyrimidin-4-amine | |
| 64 | 2-(4-fluoropiperidin-1-yl)-N-(3- | — |
| | (4-methoxyphenyl)-1H-pyrazol- | |
| | 5-yl)pyrimidin-4-amine | |
| 65 | 2-(4,4-difluoropiperidin-1-yl)- | — |
| | N-(3-(4-methoxyphenyl)-1H- | |
| | pyrazol-5-yl)pyrimidin-4-amine | |
| 66 | N-(3-(4-methoxyphenyl)-1H- | — |
| | pyrazol-5-yl)-2- | |
| | thiomorpholinopyrimidin-4- | |
| | amine | |
| 67 | 4-(4-((3-(4-methoxyphenyl)- | — |
| | 1H-pyrazol-5- | |
| | yl)amino)pyrimidin-2- | |
| | yl)thiomorpholine 1,1-dioxide | |
| 68 | N-(3-(4-methoxyphenyl)-1H- | — |
| | pyrazol-5-yl)-2-(piperidin-1- | |
| | yl)pyrimidin-4-amine | |
| 69 | N-(3-(4-methoxyphenyl)-1H- | [M + H]⁺ 365.1944/¹H NMR (400 MHz, |
| | pyrazol-5-yl)-2-(4- | DMSO-d₆): δ 0.91 (3H, d, J = 6.0 Hz); |
| | methylpiperidin-1-yl)pyrimidin- | 1.01-1.10 (2H, m); 1.58-1.66 (3H, m); 2.82 |
| | 4-amine | (2H, d, J = 11.8 Hz); 3.78 (3H, s); 4.63 |
| | | (2H, d, J = 13.2 Hz); 6.27 (1H, s); 6.70 |
| | | (1H, s); 7.02 (2H, d, J = 8.4 Hz); 7.60 (2H, |
| | | d, J = 8.4 Hz); 7.89 (1H, d, J = 5.2 Hz); |
| | | 9.47 (1H, s); 12.56 (1H, s). ¹³C NMR (100 |
| | | MHz, DMSO-d₆): δ 22.32; 31.25; 34.06; |
| | | 44.25; 55.71; 93.31; 96.03; 114.95; 122.77; |
| | | 126.74; 142.38; 149.80; 156.54; 159.51; |
| | | 160.32; 161.72. |
| 70 | N-(3-(4-methoxyphenyl)-1H- | [M + H]⁺ 419.1658/¹H NMR (400 MHz, |
| | pyrazol-5-yl)-2-(4- | DMSO-d⁶): δ 1.32-1.43 (2H, m), 1.87 (2H, |
| | (trifluoromethyl)piperidin-1- | d, J = 10.8 Hz); 2.58-2.63 (1H, m); 2.87 |
| | yl)pyrimidin-4-amine | (2H, t, J = 12.0 Hz); 3.78 (3H, s); 4.77 |
| | | (2H, d, J = 12.8 Hz); 6.35 (1H, s); 6.68 |
| | | (1H, s); 7.02 (2H, d, J = 8.8 Hz); 7.62 (2H, |
| | | d, J = 8.8 Hz); 7.92 (1H, d, J = 5.2 Hz); |
| | | 9.55 (1H, s); 12.59 (1H, s). ¹³C NMR (100 |
| | | MHz, DMSO-d₆): δ 24.24; 42.68; 55.67; |
| | | 93.18; 96.68; 114.93; 122.58; 126.76; |
| | | 129.60; 142.24; 149.57; 156.61; 159.59; |
| | | 160.29; 161.51. |
| 71 | N-(3-(4-methoxyphenyl)-1H- | — |
| | pyrazol-5-yl)-2-(4- | |
| | methylpiperazin-1- | |
| | yl)pyrimidin-4-amine | |
| 72 | N-(3-(4-methoxyphenyl)-1H- | — |
| | pyrazol-5-yl)-2-(piperazin-1- | |
| | yl)pyrimidin-4-amine | |
| 73 | N-(3-(4-methoxyphenyl)-1H- | — |
| | pyrazol-5-yl)-2-(pyrrolidin-1- | |
| | yl)pyrimidin-4-amine | |
| 77 | 5-chloro-N-(3-(4- | — |
| | methoxyphenyl)-1H-pyrazol-5- | |
| | yl)-2-morpholinopyrimidin-4- | |
| | amine | |
| 78 | 5-fluoro-N-(3-(4- | — |
| | methoxyphenyl)-1H-pyrazol-5- | |
| | yl)-2-morpholinopyrimidin-4- | |
| | amine | |
| 79 | N-(3-(4-methoxyphenyl)-1H- | — |
| | pyrazol-5-yl)-6-methyl-2- | |
| | morpholinopyrimidin-4-amine | |
| 80 | N-(3-(4-methoxyphenyl)-1H- | — |
| | pyrazol-5-yl)-4-morpholino- | |
| | 1,3,5-triazin-2-amine | |
| 81 | N-(3-(4-methoxyphenyl)-1H- | — |
| | pyrazol-5-yl)-2- | |
| | morpholinopyridin-4-amine | |
| 82 | N-(3-(4-methoxyphenyl)-1H- | — |
| | pyrazol-5-yl)-6- | |
| | morpholinopyridin-2-amine | |
| 83 | N-(3-(4-methoxyphenyl)-1H- | — |
| | pyrazol-5-yl)-6- | |
| | morpholinopyrazin-2-amine | |
| 84 | N-(3-(4-methoxyphenyl)-1H- | — |
| | pyrazol-5-yl)-N-methyl-2- | |
| | morpholinopyrimidin-4-amine | |
| 85 | N-ethyl-N-(3-(4- | — |
| | methoxyphenyl)-1H-pyrazol-5- | |
| | yl)-2-morpholinopyrimidin-4- | |
| | amine | |
| 86a | 2-(2,6-dimethylmorpholino)-N- | — |
| | (3-(3-fluoro-4-methoxyphenyl)- | |
| | 1H-pyrazol-5-yl)pyrimidin-4- | |
| | amine | |
| 86b | 2-((2R,6S)-2,6- | [M + H]⁺ 399.1791/¹H NMR (400 MHz, |
| | dimethylmorpholino)-N-(3-(3- | DMSO-d₆): δ 1.14 (6H, d, J = 6.4 Hz); |
| | fluoro-4-methoxyphenyl)-1H- | 2.45-2.51 (2H, m); 3.54-3.58 (2H, m); 3.86 |
| | pyrazol-5-yl)pyrimidin-4-amine | (3H, s); 4.47 (2H, d, J = 11.6 Hz); 6.31 |
| | | (1H, s); 6.79 (1H, s); 7.24 (1H, t, J = 8.4 |
| | | Hz); 7.46-7.55 (2H, m); 7.91 (1H, d, J = |
| | | 5.6 Hz); 9.60 (1H, s); 12.66 (1H, s). ¹³C |
| | | NMR (100 MHz, DMSO-d₆): δ 19.26; |
| | | 49.65; 56.57; 71.46; 94.11; 96.82; 112.91 |
| | | (d, ²J_{C-F} = 20.0 Hz); 114.73; 121.67; |
| | | 122.98; (d, ³J_{C-F} = 14.0 Hz); 141.08; |
| | | 147.41; 149.62; 152.05 (d, ¹J_{C-F} = 242.0 |
| | | Hz); 156.62; 160.15; 161.48. |
| 87 | N-(3-(3-fluoro-4- | [M + H]⁺ 383.1895/¹H NMR (400 MHz, |
| | methoxyphenyl)-1H-pyrazol-5- | DMSO-d₆): δ 0.90 (3H, d, J = 6.0 Hz); |
| | yl)-2-(4-methylpiperidin-1- | 1.01-1.09 (2H, m); 1.59-1.65 (3H, m); 2.81 |
| | yl)pyrimidin-4-amine | (2H, t, J = 11.6 Hz); 3.86 (3H, s); 4.62 |
| | | (2H, d, J = 12.8 Hz); 6.26 (1H, s); 6.74 |
| | | (1H, s); 7.25 (1H, t, J = 8.8 Hz); 7.44-7.55 |
| | | (2H, m); 7.89 (1H, d, J = 5.6 Hz); 9.50 |
| | | (1H, s); 12.64 (1H, s). ¹³C NMR (100 |
| | | MHz, DMSO-d₆): δ 22.32; 31.23; 34.06; |
| | | 44.24; 56.66; 95.94; 112.91 (d, ²J_{C-F} = 20.0 |
| | | Hz); 115.04; 121.83; 152.15 (d, ¹J_{C-F} = |
| | | 242.0 Hz); 156.80; 160.32; 161.70. |
| 88 | N-(3-(3-fluoro-4- | — |
| | methoxyphenyl)-1H-pyrazol-5- | |
| | yl)-2-(4-fluoropiperidin-1- | |
| | yl)pyrimidin-4-amine | |
| 89 | N-(3-(3-fluoro-4- | [M + H]⁺ 437.1614/¹H NMR (400 MHz, |
| | methoxyphenyl)-1H-pyrazol-5- | DMSO-d₆): δ 1.33-1.43 (2H, m); 1.86 (2H, |
| | yl)-2-(4- | d, J = 11.2 Hz); 2.56-2.61 (1H, m); 2.87 |
| | (trifluoromethyl)piperidin-1- | (2H, t, J = 12.0 Hz); 3.87 (3H, s); 4.76 |
| | yl)pyrimidin-4-amine | (2H, d, J = 13.6 Hz), 6.35 (1H, s); 6.71 |
| | | (1H, s); 7.25 (1H, t, J = 8.6 Hz); 7.47 (1H, |
| | | d, J = 7.6 Hz); 7.55 (1H, d, J = 12.8 Hz); |
| | | 7.92 (1H, d, J = 5.2 Hz); 9.49 (1H, s); |
| | | 12.62 (1H, s). ¹³C NMR (100 MHz, |
| | | DMSO-d₆): δ 23.80; 42.20; 56.07; 93.40; |
| | | 96.15; 112.46 (d, ²J_{C-F} = 19.9 Hz); 114.44; |
| | | 121.44; 122.52; 127.70 (q, ¹J_{C-F} = 276.5 |
| | | Hz); 140.75; 146.92 (d, ²J_{C-F} = 12.8 Hz); |
| | | 149.18; 150.36; 152.78; 156.29; 159.85; |
| | | 161.04. |
| 90 | N-(3-(3-fluoro-4- | [M + H]⁺ 427.2160/¹H NMR (400 MHz, |
| | methoxyphenyl)-1H-pyrazol-5- | DMSO-d₆): δ 1.17 (12H, s); 3.61 (4H, s); |
| | yl)-2-(2,2,6,6- | 3.86 (3H, s); 6.27 (1H, bs); 6.81 (1H, bs); |
| | tetramethylmorpholino)pyrimidin- | 7.26 (1H, t, J = 8.8 Hz); 7.44 (1H, d, J = |
| | 4-amine | 8.4 Hz); 7.51 (1H, d, J = 12.0 Hz); 7.90 |
| | | (1H, d, J = 5.6 Hz); 9.61 (1H, s); 12.66 |
| | | (1H, s). |
| 91 | 2-(3,3-dimethylmorpholino)-N- | — |
| | (3-(3-fluoro-4-methoxyphenyl)- | |
| | 1H-pyrazol-5-yl)pyrimidin-4- | |
| | amine | |
| 92 | 2-(2,2-dimethylmorpholino)-N- | [M + H]⁺ 399.1919/¹H NMR (400 MHz, |
| | (3-(3-fluoro-4-methoxyphenyl)- | DMSO-d₆): δ 1.16 (6H, s); 3.56 (2H, s); |
| | 1H-pyrazol-5-yl)pyrimidin-4- | 3.66 (4H, s); 3.86 (3H, s); 6.28 (1H, bs); |
| | amine | 6.70 (1H, bs); 7.25 (1H, t, J = 8.8 Hz); 7.46 |
| | | (1H, d, J = 8.4 Hz); 7.53 (1H, d, J = 12.8 |
| | | Hz); 7.90 (1H, d, J = 5.6 Hz); 9.57 (1H, |
| | | bs); 12.65 (1H, s). |
| 93 | 2-(3,5-dimethylmorpholino)-N- | — |
| | (3-(3-fluoro-4-methoxyphenyl)- | |
| | 1H-pyrazol-5-yl)pyrimidin-4- | |
| | amine | |
| 94 | N-(3-(3-fluoro-4- | [M + H]⁺ 385.1769/¹H-NMR (400 MHz, |
| | methoxyphenyl)-1H-pyrazol-5- | DMSO-d6): δ 1.14 (3H, d, J = 6.4 Hz); |
| | yl)-2-(2- | 2.49-2.60 (1H, m); 2.85-2.93 (1H, m); |
| | methylmorpholino)pyrimidin-4- | 3.45-3.53 (2H, m); 3.86-3.89 (4H, m); |
| | amine | 4.35-4.48 (2H, m); 6.34 (1H, s); 6.76 (1H, |
| | | s); 7.24 (1H, t, J = 8.6 Hz); 7.47 (1H, d, J = |
| | | 8.0 Hz); 7.56 (1H, dd, J = 12.4 Hz, J = 2.0 |
| | | Hz); 7.92 (1H, d, J = 5.2 Hz); 9.58 (1H, s); |
| | | 12.66 (1H, s). ¹H-NMR (400 MHz, |
| | | DMSO-d6): δ 19.26; 43.80; 50.40; 56.59; |
| | | 66.31; 71.50; 94.06; 96.87; 112.97 (d, |
| | | ²J_{C-F} = 20.0 Hz); 114.88; 121.85; 123.04 (d, |
| | | 3J_{C-F} = 8.0 Hz); 141.17; 147.41 (d, ²J_{C-F} = 10.0 |
| | | Hz); 149.63; 152.06 (d, ¹J_{C-F} = 242.0 Hz); |
| | | 156.66; 160.23; 161.70. |
| 95 | N-(3-(3-fluoro-4- | — |
| | methoxyphenyl)-1H-pyrazol-5- | |
| | yl)-2-(3- | |
| | methylmorpholino)pyrimidin-4- | |
| | amine | |
| 96 | N-(3-(3-fluoro-4- | [M + H]⁺ 439.1515/¹H-NMR (400 MHz, |
| | methoxyphenyl)-1H-pyrazol-5- | DMSO-d6): δ 2.99-3.08 (2H, m); 3.61-3.67 |
| | yl)-2-(2- | (1H, m); 3.87 (3H, s); 4.03-4.06 (1H, m); |
| | (trifluoro- | 4.26-4.28 (1H, m); 4.40-4.44 (1H, m); |
| | methyl)morpholino)pyrimidin- | 4.68-4.71 (1H, m); 6.36 (1H, s); 6.76 (1H, |
| | 4-amine | bs); 7.23 (1H, t, J = 8.8 Hz); 7.46 (1H, d, J = |
| | | 8.4 Hz); 7.52 (1H, d, J = 12.4 Hz); 7.96 |
| | | (1H, d, J = 6.0 Hz); 9.72 (1H, s); 12.69 (1H, |
| | | s). ). ¹H-NMR (400 MHz, DMSO-d6): δ |
| | | 42.34; 43.50; 56.59; 66.35; 72.24 (q, |
| | | ²J_{C-F} = 30.0 Hz); 93.95; 97.61; 112.97 (d, ¹J_{C-F} = |
| | | 19.0 Hz); 114.78; 121.76; 122.97 (d, |
| | | ³J_{C-F} = 7.0 Hz); 124.24 (q, ¹J_{C-F} = 279.0 Hz); |
| | | 141.22; 147.45 (d, ²J_{C-F} = 11.0 Hz); |
| | | 149.45; 152.06 (d, ¹J_{C-F} = 242.0 Hz); |
| | | 156.58; 160.25; 161.52. |
| 97 | N-(3-(3-fluoro-4- | — |
| | methoxyphenyl)-1H-pyrazol-5- | |
| | yl)-2-(tetrahydro-1H-furo[3,4- | |
| | c]pyrrol-5(3H)-yl)pyrimidin-4- | |
| | amine | |
| 98 | 2-(3-azabicyclo[3.1.0]hexan-3- | — |
| | yl)-N-(3-(3-fluoro-4- | |
| | methoxyphenyl)-1H-pyrazol-5- | |
| | yl)pyrimidin-4-amine | |
| 99 | N-(3-(3-fluoro-4- | — |
| | methoxyphenyl)-1H-pyrazol-5- | |
| | yl)-2-(7-azaspiro[3.5]nonan-7- | |
| | yl)pyrimidin-4-amine | |
| 100 | 2-(4,4-dimethylpiperidin-1-yl)- | — |
| | N-(3-(3-fluoro-4- | |
| | methoxyphenyl)-1H-pyrazol-5- | |
| | yl)pyrimidin-4-amine | |
| 101 | N-(3-(3-fluoro-4- | — |
| | methoxyphenyl)-1H-pyrazol-5- | |
| | yl)-2-(6-azaspiro[2.5]octan-6- | |
| | yl)pyrimidin-4-amine | |
| 102 | N-(3-(3-fluoro-4- | — |
| | methoxyphenyl)-1H-pyrazol-5- | |
| | yl)-2-(3-methyl-8- | |
| | azabicyclo[3.2.1]octan-8- | |
| | yl)pyrimidin-4-amine | |
| 103 | N-(3-(3-fluoro-4- | — |
| | methoxyphenyl)-1H-pyrazol-5- | |
| | yl)-2-(2-oxa-6- | |
| | azaspiro[3.3]heptan-6- | |
| | yl)pyrimidin-4-amine | |
| 104 | N-(3-(3-fluoro-4- | — |
| | methoxyphenyl)-1H-pyrazol-5- | |
| | yl)-2-(1-oxa-7- | |
| | azaspiro[3.5]nonan-7- | |
| | yl)pyrimidin-4-amine | |
| 105 | N-(3-(3-fluoro-4- | — |
| | methoxyphenyl)-1H-pyrazol-5- | |
| | yl)-2-(4-methylpiperazin-1- | |
| | yl)pyrimidin-4-amine | |
| 106 | 2-((2R,6R)-2,6- | ¹H NMR (400 MHz, CDCl₃): δ 1.23 (6H, |
| | dimethylmorpholino)-N-(3-(3- | d, J = 6.0 Hz); 3.48 (2H, dd, J = 12.8; 6.4 |
| | fluoro-4-methoxyphenyl)-1H- | Hz); 3.85-3.91 (5H, m); 4.07-4.11 (2H, m); |
| | pyrazol-5-yl)pyrimidin-4-amine | 6.09 (1H, dd, J = 4.0 Hz); 6.36 (1H, s); |
| | | 6.97 (1H, t, J = 8.4 Hz); 7.34-7.36 (2H, m); |
| | | 7.46 (1H, s); 8.01 (1H, d, J = 5.2 Hz). |
| 107 | 2-((2S,6S)-2,6- | — |
| | dimethylmorpholino)-N-(3-(3- | |
| | fluoro-4-methoxyphenyl)-1H- | |
| | pyrazol-5-yl)pyrimidin-4-amine | |
| 108 | N-(3-(3-fluoro-4- | — |
| | methoxyphenyl)-1H-pyrazol-5- | |
| | yl)-2-(pyrrolidin-1- | |
| | yl)pyrimidin-4-amine | |
| 109 | N-(3-(3-fluoro-4- | — |
| | methoxyphenyl)-1H-pyrazol-5- | |
| | yl)-2-(piperidin-1-yl)pyrimidin- | |
| | 4-amine | |
| 110 | 2-(azetidin-1-yl)-N-(3-(3- | — |
| | fluoro-4-methoxyphenyl)-1H- | |
| | pyrazol-5-yl)pyrimidin-4-amine | |
| 111 | N-(3-(3-fluoro-4- | — |
| | methoxyphenyl)-1H-pyrazol-5- | |
| | yl)-2-(3-methylazetidin-1- | |
| | yl)pyrimidin-4-amine | |
| 112 | N-(3-(3-fluoro-4- | — |
| | methoxyphenyl)-1H-pyrazol-5- | |
| | yl)-2-(3-fluoroazetidin-1- | |
| | yl)pyrimidin-4-amine | |
| 113 | 2-(3,3-dimethylazetidin-1-yl)- | — |
| | N-(3-(3-fluoro-4- | |
| | methoxyphenyl)-1H-pyrazol-5- | |
| | yl)pyrimidin-4-amine | |
| 114 | 2-(3-fluoro-3-methylazetidin-1- | — |
| | yl)-N-(3-(3-fluoro-4- | |
| | methoxyphenyl)-1H-pyrazol-5- | |
| | yl)pyrimidin-4-amine | |
| 115 | 2-(3,3-difluoroazetidin-1-yl)-N- | — |
| | (3-(3-fluoro-4-methoxyphenyl)- | |
| | 1H-pyrazol-5-yl)pyrimidin-4- | |
| | amine | |
| 116 | N-(3-(3-fluoro-4- | — |
| | methoxyphenyl)-1H-pyrazol-5- | |
| | yl)-2-(2-methylpyrrolidin-1- | |
| | yl)pyrimidin-4-amine | |
| 117 | N-(3-(3-fluoro-4- | — |
| | methoxyphenyl)-1H-pyrazol-5- | |
| | yl)-2-(3-methylpyrrolidin-1- | |
| | yl)pyrimidin-4-amine | |
| 118 | 2-(3,3-difluoropyrrolidin-1-yl)- | — |
| | N-(3-(3-fluoro-4- | |
| | methoxyphenyl)-1H-pyrazol-5- | |
| | yl)pyrimidin-4-amine | |
| 119 | 2-(4,4-difluoropiperidin-1-yl)- | — |
| | N-(3-(3-fluoro-4- | |
| | methoxyphenyl)-1H-pyrazol-5- | |
| | yl)pyrimidin-4-amine | |
| 120 | 2-(2-oxa-5- | — |
| | azabicyclo[4.1.0]heptan-5-yl)- | |
| | N-(3-(3-fluoro-4- | |
| | methoxyphenyl)-1H-pyrazol-5- | |
| | yl)pyrimidin-4-amine | |
| 121 | N-(3-(3-fluoro-4- | — |
| | methoxyphenyl)-1H-pyrazol-5- | |
| | yl)-2-(1,4-oxazepan-4- | |
| | yl)pyrimidin-4-amine | |
| 122 | (R)-N-(3-(3-fluoro-4- | — |
| | methoxyphenyl)-1H-pyrazol-5- | |
| | yl)-2-(2- | |
| | methylmorpholino)pyrimidin-4- | |
| | amine | |
| 123 | (S)-N-(3-(3-fluoro-4- | — |
| | methoxyphenyl)-1H-pyrazol-5- | |
| | yl)-2-(2- | |
| | methylmorpholino)pyrimidin-4- | |
| | amine | |
| 124 | 2-(azepan-1-yl)-N-(3-(3-fluoro- | — |
| | 4-methoxyphenyl)-1H-pyrazol- | |
| | 5-yl)pyrimidin-4-amine | |
| 125 | 2-(3-oxa-8- | — |
| | azabicyclo[3.2.1]octan-8-yl)-N- | |
| | (3-(3-fluoro-4-methoxyphenyl)- | |
| | 1H-pyrazol-5-yl)pyrimidin-4- | |
| | amine | |
| 126 | 2-(8-oxa-3- | — |
| | azabicyclo[3.2.1]octan-3-yl)-N- | |
| | (3-(3-fluoro-4-methoxyphenyl)- | |
| | 1H-pyrazol-5-yl)pyrimidin-4- | |
| | amine | |
| 127 | 2-((1S,4S)-2-oxa-5- | — |
| | azabicyclo[2.2.1]heptan-5-yl)- | |
| | N-(3-(3-fluoro-4- | |
| | methoxyphenyl)-1H-pyrazol-5- | |
| | yl)pyrimidin-4-amine | |
| 128 | 2-((1R,4R)-2-oxa-5- | — |
| | azabicyclo[2.2.1]heptan-5-yl)- | |
| | N-(3-(3-fluoro-4- | |
| | methoxyphenyl)-1H-pyrazol-5- | |
| | yl)pyrimidin-4-amine | |
| 129a | 2-(2,6-dimethylmorpholino)-N- | — |
| | (3-(2-fluoro-4-methoxyphenyl)- | |
| | 1H-pyrazol-5-yl)pyrimidin-4- | |
| | amine | |
| 129b | 2-((2R,6S)-2,6- | [M + H]⁺ 399.1956/¹H-NMR (400 MHz, |
| | dimethylmorpholino)-N-(3-(2- | DMSO-d6): δ 1.15 (6H, d, J = 6.4 Hz); |
| | fluoro-4-methoxyphenyl)-1H- | 3.53-3.57 (2H, m); 3.81 (3H, s); 2.45-2.48 |
| | pyrazol-5-yl)pyrimidin-4-amine | (2H, m); 4.48 (2H, m); 6.25 (1H, s); 6.88- |
| | | 6.98 (3H, m); 7.72 (1H, t, J = 9.0 Hz); 7.90 |
| | | (1H, d, J = 5.6 Hz); 9.70 (1H, s); 12.64 (1H, |
| | | s). ¹³C-NMR (100 MHz, DMSO-d6): δ |
| | | 19.20; 49.64; 56.27; 71.47; 96.31; 96.99; |
| | | 102.67 (d, ²J_{C-F} = 25.0 Hz); 110.30 (d, |
| | | ²J_{C-F} = 13.0 Hz); 111.66; 128.09; 136.05; |
| | | 149.62; 156.38; 159.57 (d, ¹J_{C-F} = 236.0 |
| | | Hz); 160.02; 160.86; 161.48. |
| 130 | N-(3-(2-fluoro-4- | [M + H]⁺ 383.1913/¹H-NMR (400 MHz, |
| | methoxyphenyl)-1H-pyrazol-5- | DMSO-d6): δ 0.92 (3H, d, J = 6.4 Hz); |
| | yl)-2-(4-methylpiperidin-1- | 1.01-2.00 (2H, m); 1.59-1.66 (3H, m); |
| | yl)pyrimidin-4-amine | 2.80-2.85 (2H, m); 3.81 (3H, s); 4.62-4.65 |
| | | (2H, m); 6.22 (1H, s); 6.90-7.00 (1H, t, J = |
| | | 8.4 Hz); 7.89 (1H, d, J = 5.6 Hz); 9.54 (1H, |
| | | s); 12.58 (1H, s). ¹³C-NMR (100 MHz, |
| | | DMSO-d6): δ 22.40; 31.29; 34.06; 44.25; |
| | | 56.26; 96.18; 96.35; 102.81 (d, ²J_{C-F} = 27.0 |
| | | Hz); 110.41 (d, ²J_{C-F} = 12.0 Hz); 111.58; |
| | | 128.16; 136.05; 149.76; 156.55; 159.57 |
| | | (¹J_{C-F} = 232.0 Hz); 160.10; 160.87; 161.65. |
| 131 | N-(3-(2-fluoro-4- | [M + H]⁺ 387.1773/¹H-NMR (400 MHz, |
| | methoxyphenyl)-1H-pyrazol-5- | DMSO-d6): δ 1.62-1.71 (2H, m); 1.84-1.95 |
| | yl)-2-(4-fluoropiperidin-1- | (2H, m); 3.63-3.69 (2H, m); 3.80 (3H, s); |
| | yl)pyrimidin-4-amine | 3.92-3.97 (2H, m); 4.81-4.97 (1H, m); 6.28 |
| | | (1H, s); 6.85-6.98 (3H, m); 7.71 (1H, t, J = |
| | | 8.8 Hz); 7.91 (1H, d, J = 5.6 Hz), 9.57 (1H, |
| | | s); 12.58 (1H, s). |
| 132 | N-(3-(2-fluoro-4- | [M + H]⁺ 437.1719/¹H-NMR (400 MHz, |
| | methoxyphenyl)-1H-pyrazol-5- | DMSO-d6): δ 1.32-1.42 (2H, m); 1.93-1.95 |
| | yl)-2-(4- | (2H, m); 2.61-2.63 (1H, m); 2.84-2.90 (2H, |
| | (trifluoromethyl)piperidin-1- | m); 3.80 (3H, s); 4.75 (2H, m); 6.27 (1H, |
| | yl)pyrimidin-4-amine | s); 6.89-7.00 (3H, m); 7.72 (1H, t, J = 8.6 |
| | | Hz); 7.92 (1H, d, J = 5.6 Hz); 9.67 (1H, s); |
| | | 12.64 (1H, s). ¹³C-NMR (100 MHz, |
| | | DMSO-d6): δ 24.24; 42.63; 52.26; 96.35; |
| | | 96.78; 102.80 (d, ²J_{C-F} = 26.0 Hz); 110.36 |
| | | (d, ²J_{C-F} = 10.0 Hz); 111.61; 128.18; 128.20 |
| | | (q, ¹J_{C-F} = 276.0 Hz); 136.12; 149.63; |
| | | 156.62; 159.59 (d, 1JC-F = 232.0 Hz); |
| | | 160.18; 160.87; 16151. |
| 133 | N-(3-(2-fluoro-4- | [M + H]⁺ 427.2208/¹H NMR (400 MHz, |
| | methoxyphenyl)-1H-pyrazol-5- | DMSO-d₆): δ 1.17 (12H, s); 3.61 (4H, s); |
| | yl)-2-(2,2,6,6- | 3.80 (3H, s); 6.18 (1H, bs); 6.88-6.98 (3H, |
| | tetramethylmorpholino)pyrimidin- | m); 7.73 (1H, t, J = 8.8 Hz); 7.88 (1H, d, |
| | 4-amine | J = 5.6 Hz); 9.71 (1H, s); 12.65 (1H, s). |
| 134 | 2-(3,3-dimethylmorpholino)-N- | — |
| | (3-(2-fluoro-4-methoxyphenyl)- | |
| | 1H-pyrazol-5-yl)pyrimidin-4- | |
| | amine | |
| 135 | 2-(2,2-dimethylmorpholino)-N- | [M + H]⁺ 399.1908/¹H NMR (400 MHz, |
| | (3-(2-fluoro-4-methoxyphenyl)- | DMSO-d₆): δ 1.17 (6H, s); 3.57 (2H, s); |
| | 1H-pyrazol-5-yl)pyrimidin-4- | 3.66 (4H, s); 3.80 (3H, s); 6.24 (1H, bs); |
| | amine | 6.88-6.98 (3H, m); 7.72 (1H, t, J = 8.8 Hz); |
| | | 7.89 (1H, d, J = 5.2 Hz); 9.65 (1H, s); |
| | | 12.63 (1H, s). |
| 136 | 2-(3,5-dimethylmorpholino)-N- | LCMS: 399.10 [M + 1]⁺ |
| | (3-(2-fluoro-4-methoxyphenyl)- | |
| | 1H-pyrazol-5-yl)pyrimidin-4- | |
| | amine | |
| 137 | N-(3-(2-fluoro-4- | [M + H]⁺ 385.1804/¹H-NMR (400 MHz, |
| | methoxyphenyl)-1H-pyrazol-5- | DMSO-d6): δ 1.15 (3H, d, J = 6.4 Hz); |
| | yl)-2-(2- | 2.55-2.61 (1H, m); 2.86-2.92 (1H, m); |
| | methylmorpholino)pyrimidin-4- | 3.45-3.53 (1H, m); 3.80 (3H, s); 3.84-3.88 |
| | amine | (1H, m); 4.36-4.39 (1H, m); 4.45-4.48 (1H, |
| | | m); 6.29 (1H, s); 6.88-6.98 (3H, m); 7.12 |
| | | (1H, t, J = 9.0 Hz); 7.91 (1H, d, J = 5.6 Hz); |
| | | 9.66 (1H, s); 12.62 (1H, s). ¹³C-NMR (100 |
| | | MHz, DMSO-d6): δ 19.19; 43.80; 50.39; |
| | | 56.27; 66.26; 71.53; 96.37 (d, ³J_{C-F} = 9.0 |
| | | Hz); 97.04; 102.73 (d, ²J_{C-F} = 26.0 Hz); |
| | | 110.34 (d, ²J_{C-F} = 13 Hz); 111.63; 128.20; |
| | | 136.09; 149.61; 156.46; 159.59 (d, ¹J_{C-F} = |
| | | 235.0 Hz); 160.10; 160.87; 161.69. |
| 138 | N-(3-(2-fluoro-4- | [M + H]⁺ 385.1773/¹H-NMR (400 MHz, |
| | methoxyphenyl)-1H-pyrazol-5- | DMSO-d6): δ 1.19 (3H, d, J = 6.8 Hz); |
| | yl)-2-(3- | 3.08-3.15 (1H, m); 3.38-3.45 (1H, m); |
| | methylmorpholino)pyrimidin-4- | 3.56-3.59 (1H, m); 3.67-3.70 (1H, m); 3.81 |
| | amine | (3H, s); 3.86-3.90 (1H, m); 4.22-4.24 (1H, |
| | | m); 4.58-4.61 (1H, m); 6.28 (1H, s); 6.88- |
| | | 6.98 (3H, m); 7.70 (1H, t, J = 8.6 Hz); 7.92 |
| | | (1H, d, J = 5.6 Hz); 9.58 (1H, s); 12.57 (1H, |
| | | s). ¹³C-NMR (100 MHz, DMSO-d6): δ |
| | | 13.84; 39.17; 46.53; 56.26; 66.80; 70.91; |
| | | 96.23 (d, ³J_{C-F} = 9.0 Hz); 96.89; 102.78 |
| | | (²J_{C-F} = 26.0 Hz); 110.38 (d, ²J_{C-F} = 14.0 |
| | | Hz); 111.60; 128.19; 136.11; 149.64; |
| | | 156.54; 159.64 (d, ¹J_{C-F} = 246.0 Hz); |
| | | 160.80 (d, ³J_{C-F} = 12.0 Hz); 161.27. |
| 139 | N-(3-(2-fluoro-4- | [M + H]⁺ 439.1563/¹H-NMR (400 MHz, |
| | methoxyphenyl)-1H-pyrazol-5- | DMSO-d6): δ 2.98-3.08 (2H, m); 3.62-3.67 |
| | yl)-2-(2- | (1H, m); 3.81 (3H, s); 4.02-4.04 (1H, m); |
| | (trifluoromethyl)morpholino)py | 4.23-4.26 (1H, m); 4.41-4.44 (1H, m); |
| | rimidin-4-amine | 4.66-4.69 (1H, m); 6.36 (1H, s); 6.80 (1H, |
| | | bs); 6.89 (1H, d, J = 8.4 Hz), 6.95 (1H, d, |
| | | J = 13.6 Hz); 7.69 (1H, t, J = 5.6 Hz); 9.77 |
| | | (1H, s); 12.65 (1H, s). ¹³C-NMR (100 |
| | | MHz, DMSO-d6): δ 42.31; 43.52; 56.28; |
| | | 66.26; 74.24 (q, ²J_{C-F} = 30.0 Hz); 96.20 (d, |
| | | ³J_{C-F} = 8.0 Hz); 97.70; 102.67 (d, ²J_{C-F} = |
| | | 26.0 Hz); 110.30 (d, ²J_{C-F} = 13.0 Hz); |
| | | 111.52; (124.16 (q, ¹J_{C-F} = 278.0 Hz); |
| | | 128.39 (d, ³J_{C-F} = 5.0 Hz); 130.29 (d, |
| | | ⁴J_{C-F} = 3.0 Hz); 149.39; 156.54; 159.66 (d, |
| | | ¹J_{C-F} = 247.0 Hz); 160.22; 160.88 (d, ³J_{C-F} = |
| | | 12.0 Hz), 161.52. |
| 149 | N-(3-(2-fluoro-4- | [M + H]⁺ 397.1707/¹H NMR (400 MHz, |
| | methoxyphenyl)-1H-pyrazol-5- | DMSO-d6): δ 2.97-2.98 (2H, m); 3.44-3.48 |
| | yl)-2-(tetrahydro-1H-furo[3,4- | (2H, m); 3.53-3.56 (2H, m); 3.65-3.70 (2H, |
| | c]pyrrol-5(3H)-yl)pyrimidin-4- | m); 3.80 (3H, s); 3.82-3.85 (2H, m); 6.25 |
| | amine | (1H, bs); 6.88-6.99 (3H, m); 7.70 (1H, t, |
| | | J = 8.8 Hz); 7.86 (1H, d, J = 5.6 Hz); 9.54 |
| | | (1H, s); 12.53 (1H, s). |
| 141 | 2-(3-azabicyclo[3.1.0]hexan-3- | [M + H]⁺ 367.1680/¹H-NMR (400 MHz, |
| | yl)-N-(3-(2-fluoro-4- | DMSO-d6): δ 0.13-0.16 (1H, m); 0.69-0.74 |
| | methoxyphenyl)-1H-pyrazol-5- | (1H, m); 1.61-1.63 (2H, m); 3.43-3.45 (2H, |
| | yl)pyrimidin-4-amine | m); 3.76-3.79 (2H, m); 3.81 (3H, s); 6.21 |
| | | (1H, s); 6.89-7.00 (3H, m); 7.71 (1H, t, J = |
| | | 8.2 Hz); 7.84 (1H, d, J = 5.6 Hz); 9.53 (1H, |
| | | s); 12.53 (1H, s). ¹³C-NMR (100 MHz, |
| | | DMSO-d6): δ 10.58; 16.05; 49.05; 56.25; |
| | | 96.39; 102.85 (d, ²J_{C-F} = 25.0 Hz); 110.51 |
| | | (d, ²J_{C-F} = 14.0 Hz); 111.54; 128.25; |
| | | 135.98; 149.86; 156.29; 159.61 (d, ¹J_{C-F} = |
| | | 246.0 Hz); 159.82; 160.75 (d, ³J_{C-F} = 12.0 |
| | | Hz); 161.21. |
| 142 | N-(3-(2-fluoro-4- | [M + H]⁺ 409.1991/¹H NMR (400 MHz, |
| | methoxyphenyl)-1H-pyrazol-5- | DMSO-d₆): δ 1.53 (4H, t, J = 5.2 Hz); |
| | yl)-2-(7-azaspiro[3.5]nonan-7- | 1.75-1.90 (6H, m); 3.65 (4H, t, J = 5.2 Hz); |
| | yl)pyrimidin-4-amine | 3.81 (3H, s); 6.22 (1H, bs); 6.88-7.00 (3H, |
| | | m); 7.71 (1H, t, J = 8.8 Hz); 7.87 (1H, d, |
| | | J = 5.6 Hz); 9.50 (1H, s); 12.55 (1H, s). |
| 143 | 2-(4,4-dimethylpiperidin-1-yl)- | [M + H]⁺ 397.2127/¹H NMR (400 MHz, |
| | N-(3-(2-fluoro-4- | DMSO-d₆): δ 0.96 (6H, s); 1.31 (4H, t, J = |
| | methoxyphenyl)-1H-pyrazol-5- | 5.6 Hz); 3.73 (4H, t, J = 5.6 Hz); 3.80 (3H, |
| | yl)pyrimidin-4-amine | s); 6.21 (1H, bs); 6.88-6.99 (3H, m); 7.70 |
| | | (1H, t, J = 8.8 Hz); 7.88 (1H, d, J = 5.6 |
| | | Hz); 9.51 (1H, s); 12.56 (1H, s). |
| 144 | N-(3-(2-fluoro-4- | [M + H]⁺ 395.1862/¹H-NMR (400 MHz, |
| | methoxyphenyl)-1H-pyrazol-5- | DMSO-d6): δ 0.34 (4H, bs); 1.32-1.35 |
| | yl)-2-(6-azaspiro[2.5]octan-6- | (4H, m); 3.78-3.81 (4H, m); 3.80 (3H, s); |
| | yl)pyrimidin-4-amine | 6.23 (1H, s); 6.87-6.97 (3H, m); 7.70 (1H, |
| | | t, J = 8.6 Hz); 7.89 (1H, d, J = 5.2 Hz); 9.51 |
| | | (1H, s); 12.55 (1H, s). ¹³C-NMR (100 |
| | | MHz, DMSO-d6): δ 11.68; 18.56; 34.90; |
| | | 44.07; 56.24; 96.21; 102.77 (d, ²J_{C-F} = 26.0 |
| | | Hz); 110.40 (d, ²J_{C-F} = 12.0 Hz); 111.57; |
| | | 128.15; 136.05; 149.76; 156.57; 159.64 (d, |
| | | ¹J_{C-F} = 245.0 Hz), 160.12; 160.76 (d, |
| | | ³J_{C-F} = 11.0 Hz); 161.69. |
| 145 | N-(3-(2-fluoro-4- | [M + H]⁺ 409.1933/¹H NMR (400 MHz, |
| | methoxyphenyl)-1H-pyrazol-5- | DMSO-d₆): δ 0.76-2.25 (12H, m), 3.80 |
| | yl)-2-(3-methyl-8- | (3H, s), 4.58 (2H, bs); 6.19 (1H, bs); 6.89 |
| | azabicyclo[3.2.1]octan-8- | (1H, d, J = 8.6 Hz); 6.96-6.99 (1H, m); |
| | yl)pyrimidin-4-amine | 7.71 (1H, t, J = 8.6 Hz); 7.86-7.88 (1H, m); |
| | | 9.53 (1H, s); 12.54 (1H, s). |
| 146 | N-(3-(2-fluoro-4- | [M + H]⁺/¹H NMR (500 MHz, DMSO-d₆): |
| | methoxyphenyl)-1H-pyrazol-5- | δ ¹³C NMR (100 MHz, DMSO-d₆): δ 3.82 |
| | yl)-2-(2-oxa-6- | (3H, s); 4.72-4.73 (4H, m); 6.34 (1H, s); |
| | azaspiro[3.3]heptan-6- | 6.91-7.04 (3H, m); 7.73 (1H, t, J = 7.5 Hz); |
| | yl)pyrimidin-4-amine | 7.89 (1H, d, J = 5.3 Hz); 9.72 (1H, s); 12.60 |
| | | (1H, s). |
| 147 | N-(3-(2-fluoro-4- | — |
| | methoxyphenyl)-1H-pyrazol-5- | |
| | yl)-2-(1-oxa-7- | |
| | azaspiro[3.5]nonan-7- | |
| | yl)pyrimidin-4-amine | |
| 148 | N-(3-(2-fluoro-4- | ¹H NMR (400 MHz, DMSO-d₆): δ 2.19 |
| | methoxyphenyl)-1H-pyrazol-5- | (3H, s); 2.32 (4H, t, J = 4.8 Hz); 3.69 (4H, |
| | yl)-2-(4-methylpiperazin-1- | t, J = 4.8 Hz); 3.79 (3H, s); 6.25 (1H, s); |
| | yl)pyrimidin-4-amine | 6.87-6.98 (3H, m); 7.71 (1H, t, J = 8.2 Hz); |
| | | 7.89 (1H, d, J = 5.2 Hz); 9.60 (1H, s); |
| | | 12.59 (1H, s). ¹³C NMR (100 MHz, |
| | | DMSO-d₆): δ 43.91; 46.37; 54.97; 56.25; |
| | | 96.35; 96.70; 102.78 (d, ²J_{C-F} = 25.3 Hz); |
| | | 110.40; 111.60; 128.20; 136.08; 149.65; |
| | | 156.54; 159.66 (d, ¹J_{C-F} = 246.0 Hz); |
| | | 160.72; 161.77. |
| 149 | 2-((2R,6R)-2,6- | ¹H NMR (400 MHz, CDCl₃): δ 1.25 (6H, |
| | dimethylmorpholino)-N-(3-(2- | d, J = 6.4 Hz); 3.51 (2H, dd, J = 12.8; 6.0 |
| | fluoro-4-methoxyphenyl)-1H- | Hz); 3.84 (3H, s); 3.90 (2H, dd, J = 12.8; |
| | pyrazol-5-yl)pyrimidin-4-amine | 3.2 Hz); 4.08-4.12 (2H, m); 6.18 (1H, d, |
| | | J = 4.8 Hz); 6.68 (1H, s); 6.72 (1H, dd, J = |
| | | 13.2; 2.4 Hz); 6.78 (1H, dd, J = 8.8; 2.4 |
| | | Hz); 7.36 (1H, s); 7.52-7.57 (1H, m); 8.02 |
| | | (1H, d, J = 5.6 Hz). |
| 150 | 2-((2S,6S)-2,6- | [M + H]⁺ 399.1933/¹H NMR (400 MHz, |
| | dimethylmorpholino)-N-(3-(2- | CDCl₃): δ 1.25 (6H, d, J = 6.0 Hz); 3.51 |
| | fluoro-4-methoxyphenyl)-1H- | (2H, dd, J = 12.8; 6.0 Hz); 3.84 (3H, s); |
| | pyrazol-5-yl)pyrimidin-4-amine | 3.90 (2H, dd, J = 12.8; 3.6 Hz); 4.08-4.12 |
| | | (2H, m); 6.18 (1H, d, J = 5.2 Hz); 6.67 |
| | | (1H, s); 6.72 (1H, dd, J = 13.2; 2.8 Hz); |
| | | 6.77 (1H, dd, J = 8.4; 2.8 Hz); 7.37 (1H, s); |
| | | 7.52-7.57 (1H, m); 8.02 (1H, d, J = 6.0 Hz). |
| 151 | N-(3-(2-fluoro-4- | LCMS: 355.30 [M + 1]⁺ |
| | methoxyphenyl)-1H-pyrazol-5- | |
| | yl)-2-(pyrrolidin-1- | |
| | yl)pyrimidin-4-amine | |
| 152 | N-(3-(2-fluoro-4- | LCMS: 369.30 [M + 1]⁺ |
| | methoxyphenyl)-1H-pyrazol-5- | |
| | yl)-2-(piperidin-1-yl)pyrimidin- | |
| | 4-amine | |
| 153 | 2-(azetidin-1-yl)-N-(3-(2- | LCMS: 341.30 [M + 1]⁺ |
| | fluoro-4-methoxyphenyl)-1H- | |
| | pyrazol-5-yl)pyrimidin-4-amine | |
| 154 | N-(3-(2-fluoro-4- | — |
| | methoxyphenyl)-1H-pyrazol-5- | |
| | yl)-2-(3-methylazetidin-1- | |
| | yl)pyrimidin-4-amine | |
| 155 | N-(3-(2-fluoro-4- | — |
| | methoxyphenyl)-1H-pyrazol-5- | |
| | yl)-2-(3-fluoroazetidin-1- | |
| | yl)pyrimidin-4-amine | |
| 156 | 2-(3,3-dimethylazetidin-1-yl)- | — |
| | N-(3-(2-fluoro-4- | |
| | methoxyphenyl)-1H-pyrazol-5- | |
| | yl)pyrimidin-4-amine | |
| 157 | 2-(3-fluoro-3-methylazetidin-1- | — |
| | yl)-N-(3-(2-fluoro-4- | |
| | methoxyphenyl)-1H-pyrazol-5- | |
| | yl)pyrimidin-4-amine | |
| 158 | 2-(3,3-difluoroazetidin-1-yl)-N- | — |
| | (3-(2-fluoro-4-methoxyphenyl)- | |
| | 1H-pyrazol-5-yl)pyrimidin-4- | |
| | amine | |
| 159 | N-(3-(2-fluoro-4- | LCMS: 369.30 [M + 1]⁺ |
| | methoxyphenyl)-1H-pyrazol-5- | |
| | yl)-2-(2-methylpyrrolidin-1- | |
| | yl)pyrimidin-4-amine | |
| 160 | N-(3-(2-fluoro-4- | LCMS: 369.30 [M + 1]⁺ |
| | methoxyphenyl)-1H-pyrazol-5- | |
| | yl)-2-(3-methylpyrrolidin-1- | |
| | yl)pyrimidin-4-amine | |
| 161 | 2-(3,3-difluoropyrrolidin-1-yl)- | — |
| | N-(3-(2-fluoro-4- | |
| | methoxyphenyl)-1H-pyrazol-5- | |
| | yl)pyrimidin-4-amine | |
| 162 | 2-(4,4-difluoropiperidin-1-yl)- | — |
| | N-(3-(2-fluoro-4- | |
| | methoxyphenyl)-1H-pyrazol-5- | |
| | yl)pyrimidin-4-amine | |
| 163 | 2-(2-oxa-5- | — |
| | azabicyclo[4.1.0]heptan-5-yl)- | |
| | N-(3-(2-fluoro-4- | |
| | methoxyphenyl)-1H-pyrazol-5- | |
| | yl)pyrimidin-4-amine | |
| 164 | N-(3-(2-fluoro-4- | — |
| | methoxyphenyl)-1H-pyrazol-5- | |
| | yl)-2-(1,4-oxazepan-4- | |
| | yl)pyrimidin-4-amine | |
| 165 | (R)-N-(3-(2-fluoro-4- | — |
| | methoxyphenyl)-1H-pyrazol-5- | |
| | yl)-2-(2- | |
| | methylmorpholino)pyrimidin-4- | |
| | amine | |
| 166 | (S)-N-(3-(2-fluoro-4- | — |
| | methoxyphenyl)-1H-pyrazol-5- | |
| | yl)-2-(2- | |
| | methylmorpholino)pyrimidin-4- | |
| | amine | |
| 167 | 2-(azepan-1-yl)-N-(3-(2-fluoro- | LCMS: 383.30 [M + 1]⁺ |
| | 4-methoxyphenyl)-1H-pyrazol- | |
| | 5-yl)pyrimidin-4-amine | |
| 168 | 2-(3-oxa-8- | — |
| | azabicyclo[3.2.1]octan-8-yl)-N- | |
| | (3-(2-fluoro-4-methoxyphenyl)- | |
| | 1H-pyrazol-5-yl)pyrimidin-4- | |
| | amine | |
| 169 | 2-(8-oxa-3- | — |
| | azabicyclo[3.2.1]octan-3-yl)-N- | |
| | (3-(2-fluoro-4-methoxyphenyl)- | |
| | 1H-pyrazol-5-yl)pyrimidin-4- | |
| | amine | |
| 170 | 2-((15,4S)-2-oxa-5- | — |
| | azabicyclo[2.2.1]heptan-5-yl)- | |
| | N-(3-(2-fluoro-4- | |
| | methoxyphenyl)-1H-pyrazol-5- | |
| | yl)pyrimidin-4-amine | |
| 171 | 2-((1R,4R)-2-oxa-5- | — |
| | azabicyclo[2.2.1]heptan-5-yl)- | |
| | N-(3-(2-fluoro-4- | |
| | methoxyphenyl)-1H-pyrazol-5- | |
| | yl)pyrimidin-4-amine | |
| 174 | 4-(4-methoxyphenyl)-N-(2- | [M + H]⁺ 370.1331/¹H-NMR (400 MHz, |
| | morpholinopyrimidin-4- | DMSO-d6): δ 3.68-3.70 (4H, m); 3.78-3.80 |
| | yl)thiazol-2-amine | (7H, m); 6.30 (1H, d, J = 9.0 Hz); 6.98 (2H, |
| | | d, J = 5.4 Hz); 7.38 (1H, s); 7.83 (2H, d, J = |
| | | 5.4 Hz); 7.38 (1H, s); 7.83 (2H, d, J = 9.0 |
| | | Hz); 8.08 (1H, d, J = 5.4 Hz); 11.57 (1H, s). |
| | | ¹³C-NMR (100 MHz, DMSO-d6): δ 44.42; |
| | | 55.13; 66.00; 96.93; 105.49; 114.04; |
| | | 126.98; 127.32; 148.84; 156.85; 157.36; |
| | | 157.90; 158.90; 160.90. |
| 177 | 4-(4-methoxyphenyl)-N-(2- | [M + H]⁺ 357.1529/¹H-NMR (400 MHz, |
| | morpholinopyrimidin-4- | DMSO-d6): δ 3.64-3.70 (8H, m); 3.78 (3H, |
| | yl)oxazol-2-amine | s); 6.99 (2H, d, J = 8.8 Hz); 7.34 (1H, d, J = |
| | | 5.8 Hz); 7.69 (2H, d, J = 8.8 Hz); 8.14 (1H, |
| | | s); 8.25 (1H, d, J = 5.8 Hz); 10.84 (1H, s). |
| | | ¹³C-NMR (100 MHz, DMSO-d6): δ 43.83; |
| | | 55.13; 65.98; 96.62; 114.12; 123.55; |
| | | 126.31; 128.14; 138.88; 154.92; 158.12; |
| | | 158.54; 158.94; 161.03. |
| 181 | N-(3-(4-methoxyphenyl)-4- | [M + H]⁺ 367.1745/¹H-NMR (400 MHz, |
| | methyl-1H-pyrazol-5-yl)-2- | DMSO-d6): δ 2.00 (3H, s); 3.62 (8H, bs); |
| | morpholinopyrimidin-4-amine | 3.81 (3H, s); 6.16 (1H, d, J = 5.4 Hz); 7.06 |
| | | (2H, d, J = 8.6 Hz); 7.33 (2H, d, J = 8.6 Hz); |
| | | 7.91 (1H, d, J = 5.4 Hz); 8.65 (1H, s); 12.51 |
| | | (1H, s); ¹³C-NMR (100 MHz, DMSO-d6): |
| | | δ 9.20; 44.38; 55.68; 66.57; 95.61; 105.45; |
| | | 114.80; 123.26; 128.48; 139.74; 147.49; |
| | | 156.93; 159.36; 161.76; 162.26. |
| 184 | N-(3-(4-methoxyphenyl)-1- | [M + H]⁺ 367.1782/¹H NMR (400 MHz, |
| | methyl-1H-pyrazol-5-yl)-2- | DMSO-d6): δ 3.63 (8H, s); 3.70 (3H, s); |
| | morpholinopyrimidin-4-amine | 3.77 (8H, s); 6.12 (1H, d, J = 5.6 Hz); 6.59 |
| | | (1H, s); 6.95 (2H, d, J = 8.8 Hz); 7.68 (2H, |
| | | d, J = 8.8 Hz); 7.99 (1H, d, J = 5.6 Hz); |
| | | 9.35 (1H, s). ¹³C NMR (100 MHz, DMSO- |
| | | d₆): δ 36.02; 44.53; 55.61; 66.40; 96.11; |
| | | 96.31; 114.53; 126.52; 126.80; 138.90; |
| | | 148.65; 159.27; 161.39. |
| 187 | 2-((2R,6S)-2,6- | LCMS: 413.35 [M + 1]⁺ |
| | dimethylmorpholino)-N-(3-(2- | |
| | fluoro-4-methoxyphenyl)-1- | |
| | methyl-1H-pyrazol-5- | |
| | yl)pyrimidin-4-amine | |
| 190 | 2-((2R,6S)-2,6- | LCMS: 413.35 [M + 1]⁺ |
| | dimethylmorpholino)-N-(5-(2- | |
| | fluoro-4-methoxyphenyl)-1- | |
| | methyl-1H-pyrazol-3- | |
| | yl)pyrimidin-4-amine | |
| 191a | 4-(5-((2-(2,6- | — |
| | dimethylmorpholino)pyrimidin- | |
| | 4-yl)amino)-1H-pyrazol-3- | |
| | yl)benzonitrile | |
| 191b | 4-(5-((2-((2R,6S)-2,6- | [M + H]⁺ 376.1772/¹H NMR (400 MHz, |
| | dimethylmorpholino)pyrimidin- | DMSO-d₆): δ 1.13 (6H, d, J = 6.4 Hz); |
| | 4-yl)amino)-1H-pyrazol-3- | 2.45-2.50 (2H, m); 3.53-3.57 (2H, m); 4.47 |
| | yl)benzonitrile | (2H, d, J = 12.8 Hz); 6.06 (1H, bs); 6.34 |
| | | (1H, s); 6.63 (1H, bs); 6.97 (1H, s); 7.85- |
| | | 7.94 (5H, m); 9.63 (1H, s); 10.15 (1H, s); |
| | | 13.00 (1H, s). ¹³C NMR (100 MHz, |
| | | DMSO-d₆): δ 19.28; 49.68; 71.46, 95.77; |
| | | 96.82; 110.73; 119.15; 125.81; 133.54; |
| | | 134.03; 140.48; 149.98; 156.78; 160.17; |
| | | 161.50. |
| 192a | N-(3-(3-chloro-4- | — |
| | (trifluoromethoxy)phenyl)-1H- | |
| | pyrazol-5-yl)-2-(2,6- | |
| | dimethylmorpholino)pyrimidin- | |
| | 4-amine | |
| 192b | N-(3-(3-chloro-4- | ¹H NMR (400 MHz, DMSO-d₆): δ 1.14 |
| | (trifluoromethoxy)phenyl)-1H- | (6H, d, J = 6.4 Hz); 2.44-2.52 (2H, m); |
| | pyrazol-5-yl)-2-((2R,6S)-2,6- | 3.53-3.57 (2H, m); 4.45-4.48 (2H, m); 6.31 |
| | dimethylmorpholino)pyrimidin- | (1H, s); 6.94 (1H, s); 7.58-7.99 (4H, m); |
| | 4-amine | 9.67 (1H, s); 12.92 (1H, s). |
| 193 | 2-(2,6-dimethylmorpholino)-N- | — |
| | (3-(4-(fluoromethoxy)phenyl)- | |
| | 1H-pyrazol-5-yl)pyrimidin-4- | |
| | amine | |
| 194 | 2-(2,6-dimethylmorpholino)-N- | — |
| | (3-(4-(fluoromethyl)phenyl)- | |
| | 1H-pyrazol-5-yl)pyrimidin-4- | |
| | amine | |
| 195 | N-(3-(4- | — |
| | (difluoromethyl)phenyl)-1H- | |
| | pyrazol-5-yl)-2-(2,6- | |
| | dimethylmorpholino)pyrimidin- | |
| | 4-amine | |
| 196 | 2-((2R,6S)-2,6- | ¹H NMR (400 MHz, DMSO-d₆): δ 1.13 |
| | dimethylmorpholino)-N-(5-(4- | (6H, d, J = 6.0 Hz); 2.45-2.51 (2H, m); |
| | fluorophenyl)-1H-pyrazol-3- | 3.53-3.57 (2H, m); 4.46-4.49 (2H, m); 6.34 |
| | yl)pyrimidin-4-amine | (1H, s); 6.80 (1H, s); 7.30 (2H, t, J = 8.8 |
| | | Hz); 7.72 (2H, dd, J = 8.0; 5.6 Hz); 7.92 |
| | | (1H, d, J = 5.6 Hz); 9.62 (1H, s); 12.75 |
| | | (1H, s). |
| 197 | N-(3-(4-chlorophenyl)-1H- | [M + H]⁺ 385.1548/¹H NMR (500 MHz, |
| | pyrazol-5-yl)-2-((2R,6S)-2,6- | DMSO-d₆): δ 1.15 (6H, d, J = 6.2 Hz); |
| | dimethylmorpholino)pyrimidin- | 2.47-2.52 (2H, m); 3.55-3.59 (2H, m); |
| | 4-amine | 4.48-4.50 (2H, m); 6.36 (1H, s); 6.86 (1H, |
| | | s); 7.53 (2H, d, J = 8.0 Hz); 7.83 (2H, d, |
| | | J = 8.0 Hz); 7.94 (1H, d, J = 5.3 Hz); 9.64 |
| | | (1H, s); 12.83 (1H, s). ¹³C NMR (100 |
| | | MHz, DMSO-d₆): δ 19.27; 49.68; 71.46; |
| | | 94.67; 96.81; 126.98; 128.80; 129.53; |
| | | 133.04; 141.06; 149.77; 156.70; 160.18; |
| | | 161.49. |
| 198 | 2-((2R,6S)-2,6- | [M + H]⁺ 365.2089/¹H NMR (400 MHz, |
| | dimethylmorpholino)-N-(3-(p- | DMSO-d₆): δ 1.14 (6H, d, J = 6.0 Hz); 2.31 |
| | tolyl)-1H-pyrazol-5- | (3H, s); 2.45-2.51 (2H, m); 3.53-3.57 (2H, |
| | yl)pyrimidin-4-amine | m); 4.46-4.48 (2H, m); 6.31(1H, s); 6.80 |
| | | (1H, s); 7.24 (2H, d, J = 7.8 Hz); 7.56 (2H, |
| | | d, J = 7.8 Hz); 7.91 (1H, d, J = 6.0 Hz); |
| | | 9.62 (1H, s); 12.67 (1H, s). ¹³C NMR (100 |
| | | MHz, DMSO-d₆): δ 19.26; 21.26; 49.68; |
| | | 71.46; 93.92; 96.85; 125.17; 127.20; |
| | | 130.02; 138.06; 142.21; 149.58, 156.58; |
| | | 160.15; 161.49. |
| 199 | 2-((2R,6S)-2,6- | [M + H]⁺ 379.2253/¹H NMR (400 MHz, |
| | dimethylmorpholino)-N-(3-(4- | DMSO-d₆): δ 1.13-1.19 (9H, m); 2.45-2.51 |
| | ethylphenyl)-1H-pyrazol-5- | (2H, m); 2.61 (2H, q, J = 7.6 Hz); 3.54- |
| | yl)pyrimidin-4-amine | 3.57 (2H, m); 4.46-4.49 (2H, m); 6.32 (1H, |
| | | s); 6.80 (1H, s); 7.27 (2H, d, J = 7.8 Hz); |
| | | 7.59 (2H, d, J = 7.8 Hz); 7.91 (2H, d, J = |
| | | 6.0 Hz); 9.62 (1H, s); 12.68 (1H, s). ¹³C |
| | | NMR (100 MHz, DMSO-d₆): δ 15.95; |
| | | 19.26; 28.36; 49.68; 71.46; 93.91; 96.85; |
| | | 125.27; 127.46; 128.84; 142.24; 144.24; |
| | | 144.36; 149.59; 156.60; 160.15; 161.49. |
| 200 | N-(3-(4-(1,1- | [M + H]⁺ 415.2047/¹H NMR (400 MHz, |
| | difluoroethyl)phenyl)-1H- | DMSO-d₆): δ 1.15 (6H, d, J = 6.4 Hz); 1.98 |
| | pyrazol-5-yl)-2-((2R,6S)-2,6- | (3H, t, J = 18.8 Hz); 2.46-2.53 (2H, m); |
| | dimethylmorpholino)pyrimidin- | 3.54-3.58 (2H, m); 4.47-4.50 (2H, m); 6.34 |
| | 4-amine | (1H, s), 6.91 (1H, s); 7.64 (2H, d, J = 8.2 |
| | | Hz); 7.79 (2H, d, J = 8.2 Hz); 7.92 (1H, d, |
| | | J = 5.2 Hz); 9.67 (1H, s); 12.89 (1H, s). |
| 201 | N-(3-(4- | [M + H]⁺ 417.1845/¹H NMR (400 MHz, |
| | (difluoromethoxy)phenyl)-1H- | DMSO-d₆): δ 1.13 (6H, d, J = 6.0 Hz); |
| | pyrazol-5-yl)-2-((2R,6S)-2,6- | 2.44-2.51 (2H, m); 3.52-3.57 (2H, m); |
| | dimethylmorpholino)pyrimidin- | 4.45-4.48 (2H, m); 6.33 (1H, s); 6.80 (1H, |
| | 4-amine | s); 7.25 (2H, d, J = 8.6 Hz); 7.27 (1H, t, J = |
| | | 74.0 Hz); 7.72 (2H, d, J = 8.6 Hz); 7.91 |
| | | (1H, d, J = 5.6 Hz); 9.60 (1H, s), 12.74 |
| | | (1H, s). ¹³C NMR (100 MHz, DMSO-d₆): δ |
| | | 19.27; 49.66; 71.46; 94.34; 96.82; 116.75 |
| | | (t, ¹J_{C-F} = 256.0 Hz); 119.65; 126.96; |
| | | 127.03; 141.28; 149.71; 151.10; 156.66; |
| | | 160.16; 161.48. |
| 202 | N-(3-(4- | [M + H]⁺ 401.1895/¹H NMR (400 MHz, |
| | (difluoromethyl)phenyl)-1H- | DMSO-d₆): δ 1.16 (6H, d, J = 6.0 Hz); |
| | pyrazol-5-yl)-2-((2R,6S)-2,6- | 2.47-2.54 (2H, m); 3.55-3.60 (2H, m); |
| | dimethylmorpholino)pyrimidin- | 4.47-4.51 (2H, m); 6.35 (1H, s); 6.92 (1H, |
| | 4-amine | s); 7.05 (1H, t, J = 56.0 Hz); 7.65 (2H, d, |
| | | J = 7.4 Hz); 7.83 (2H, d, J = 7.4 Hz); 7.94 |
| | | (1H, d, J = 5.6 Hz); 9.62 (1H, s); 12.88 |
| | | (1H, s). ¹³C NMR (100 MHz, DMSO-d₆): δ |
| | | 19.26; 49.70; 71.47; 115.18 (t, ¹J_{C-F} = |
| | | 234.0 Hz); 95.04; 96.84; 125.56; 126.93 (t, |
| | | ³J_{C-F} = 6.0 Hz); 132.27; 133.91 (t, ²J_{C-F} = |
| | | 22.0 Hz); 141.24; 149.80; 156.70; 160.18; |
| | | 161.51. |
| 203 | 2-((2R,6S)-2,6- | [M + H]⁺ 441.2266/¹H NMR (400 MHz, |
| | dimethylmorpholino)-N-(3- | DMSO-d₆): δ 1.14 (6H, d J = 6.0 Hz); |
| | (3,4,5-trimethoxyphenyl)-1H- | 2.47-2.53 (2H, m); 3.55-3.59 (2H, m); 3.69 |
| | pyrazol-5-yl)pyrimidin-4-amine | (3H, s); 3.84 (6H, s); 4.49-4.52 (2H, m); |
| | | 6.30 (1H, s); 6.83 (1H, s); 7.02 (2H, s); |
| | | 7.92 (1H, d, J = 5.6 Hz); 9.57 (1H, s); |
| | | 12.65 (1H, s). ¹³C NMR (100 MHz, |
| | | DMSO-d₆): δ 19.25; 49.62; 56.46; 60.59; |
| | | 71.47; 94.06; 96.87; 102.97; 125.55; |
| | | 137.91; 142.36; 149.53; 153.77; 156.47; |
| | | 160.22; 161.52. |
| 204 | N-(3-(2,3-dihydrobenzofuran-5- | [M + H]⁺ 393.2043/¹H NMR (500 MHz, |
| | yl)-1H-pyrazol-5-yl)-2- | DMSO-d₆): δ 1.16 (6H, d, J = 6.2 Hz); |
| | ((2R,6S)-2,6- | 2.48-2.51 (2H, m); 3.21 (2H, t, J = 8.7 Hz); |
| | dimethylmorpholino)pyrimidin- | 3.56-3.59 (2H, m); 4.48-4.51 (2H, m); 4.57 |
| | 4-amine | (2H, t, J = 8.7 Hz); 6.35 (1H, s); 6.73 (1H, |
| | | s); 6.84 (1H, d, J = 8.2 Hz); 7.44 (1H, d, |
| | | J = 8.2 Hz); 7.93 (1H, d, J = 5.5 Hz); 9.58 |
| | | (1H, s); 12.53 (1H, s). ¹³C NMR (100 |
| | | MHz, DMSO-d₆): δ 19.23; 29.33; 49.68; |
| | | 71.47; 71.67; 93.40; 96.34; 109.72; 122.35; |
| | | 122.58; 125.22; 128.71; 142.53; 149.51; |
| | | 156.53; 160.15; 161.49. |
| 205 | N-(3-(benzofuran-5-yl)-1H- | [M + H]⁺ 391.1896/¹H NMR (400 MHz, |
| | pyrazol-5-yl)-2-((2R,6S)-2,6- | DMSO-d₆): δ 1.14 (6H, d, J = 6.0 Hz); |
| | dimethylmorpholino)pyrimidin- | 2.46-2.52 (2H, m); 3.54-3.56 (2H, m); |
| | 4-amine | 4.47-4.50 (2H, m); 6.36 (1H, s); 6.83 (1H, |
| | | s); 6.97 (1H, s); 7.61-7.68 (2H, m); 7.91- |
| | | 8.03 (3H, m); 9.59 (1H, s); 12.71 (1H, |
| | | s). ¹³C NMR (100 MHz, DMSO-d₆): δ |
| | | 19.25; 49.69; 71.48; 94.24; 96.85; 107.29; |
| | | 112.34; 118.09; 122.16; 125.26; 128.27; |
| | | 142.56; 147.59; 149.65; 154.47; 156.63; |
| | | 160.19; 161.50. |
| 206 | 2-((2R,6S)-2,6- | [M + H]⁺ 429.1725/¹H NMR (400 MHz, |
| | dimethylmorpholino)-N-(3-(4- | DMSO-d₆): δ 1.14 (6H, d, J = 6.4 Hz); |
| | (methylsulfonyl)phenyl)-1H- | 2.46-2.52 (2H, m); 3.24 (3H, s); 3.54-3.57 |
| | pyrazol-5-yl)pyrimidin-4-amine | (2H, m); 4.46-4.49 (2H, m); 6.33 (1H, s); |
| | | 6.99 (1H, s); 7.92-7.99 (5H, m); 9.72 (1H, |
| | | s); 13.05 (1H, s). ¹³C NMR (100 MHz, |
| | | DMSO-d6): δ 19.30; 43.92; 49.68; 71.47; |
| | | 95.69; 96.84; 125.81; 128.31; 134.45; |
| | | 140.27; 140.54; 149.95; 156.75; 160.18; |
| | | 161.51. |
| 207 | 2-((2R,6S)-2,6- | [M + H]⁺ 449.1921/¹H NMR (400 MHz, |
| | dimethylmorpholino)-N-(3-(4- | DMSO-d₆): δ 1.13 (6H, d, J = 6.0 Hz); |
| | methoxy-3- | 2.45-2.50 (2H, m); 3.53-3.57 (2H, m); |
| | (trifluoromethyl)phenyl)-1H- | 4.45-4.48 (2H, m); 6.28 (1H, s); 6.84 (1H, |
| | pyrazol-5-yl)pyrimidin-4-amine | s); 7.36 (1H, d, J = 8.4 Hz); 7.89-7.94 (3H, |
| | | m); 9.63 (1H, s); 12.77 (1H, s). ¹³C NMR |
| | | (100 MHz, DMSO-d₆): δ 19.18; 49.66; |
| | | 56.86; 71.45; 93.98; 96.89; 114.14; 117.87 |
| | | (q, ²J_{C-F} = 31.0 Hz); 122.39; 123.63; 123.99 |
| | | (q, ¹J_{C-F} = 271.0 Hz); 130.85; 140.81; |
| | | 149.72; 156.57; 157.13; 160.13; 161.50. |
| 208 | N-(3-(4-bromophenyl)-1H- | [M + H]⁺ 429.1050/¹H NMR (400 MHz, |
| | pyrazol-5-yl)-2-((2R,6S)-2,6- | DMSO-d₆): δ 1.15 (6H, d, J = 8.2 Hz); |
| | dimethylmorpholino)pyrimidin- | 2.45-2.53 (2H, m); 3.51-3.59 (2H, m); |
| | 4-amine | 4.45-4.50 (2H, m); 6.35 (1H, s); 6.85 (1H, |
| | | s); 7.65-7.70 (4H, m); 7.93 (1H, d, J = 7.6 |
| | | Hz); 9.63 (1H, s); 12.83 (1H, s). ¹³C NMR |
| | | (100 MHz, DMSO-d₆): δ 19.27; 49.69; |
| | | 71.45; 94.67; 96.80; 121.59; 127.26; |
| | | 129.15; 132.43; 141.10; 149.78; 156.72; |
| | | 160.19; 161.50. |
| 209 | 2-((2R,6S)-2,6- | ¹H NMR (400 MHz, DMSO-d₆): δ 1.14 |
| | dimethylmorpholino)-N-(3-(4- | (6H, d, J = 6.4 Hz); 1.92-1.95 (4H, m); |
| | (pyrrolidin-1-yl)phenyl)-1H- | 2.45-2.51 (2H, m); 3.22-3.25 (4H, m); |
| | pyrazol-5-yl)pyrimidin-4-amine | 3.53-3.57 (2H, m); 4.47-4.50 (2H, m); 6.32 |
| | | (1H, s); 6.55 (2H, d, J = 8.6 Hz); 6.62 (1H, |
| | | s); 7.47 (2H, d, J = 8.6 Hz); 7.89 (1H, d, |
| | | J = 6.0 Hz); 9.54 (1H, s); 12.38 (1H, s). ¹³C |
| | | NMR (100 MHz, DMSO-d₆): δ 19.27; |
| | | 25.42; 47.74; 49.72; 71.46; 92.27; 96.82; |
| | | 112.16; 116.97; 126.29; 143.15; 147.90; |
| | | 149.44; 156.51; 160.21; 161.50. |
| 210 | 2-((2R,6S)-2,6- | LCMS: 399.30 [M + 1]⁺ |
| | dimethylmorpholino)-N-(3-(4- | |
| | (fluoromethoxy)phenyl)-1H- | |
| | pyrazol-5-yl)pyrimidin-4-amine | |
| 211 | N-(5-(2,3-dihydro-1H-inden-5- | LCMS: 391.35 [M + 1]⁺ |
| | yl)-1H-pyrazol-3-yl)-2- | |
| | ((2R,6S)-2,6- | |
| | dimethylmorpholino)pyrimidin- | |
| | 4-amine | |
| 212 | 2-((2R,6S)-2,6- | — |
| | dimethylmorpholino)-N-(3-(4- | |
| | (fluoromethyl)phenyl)-1H- | |
| | pyrazol-5-yl)pyrimidin-4-amine | |
| 213 | N-(3-(2,6-difluoro-4- | — |
| | methoxyphenyl)-1H-pyrazol-5- | |
| | yl)-2-((2R,6S)-2,6- | |
| | dimethylmorpholino)pyrimidin- | |
| | 4-amine | |
| 214 | 2-((2R,6S)-2,6- | — |
| | dimethylmorpholino)-N-(3-(4- | |
| | methoxy-2-methylphenyl)-1H- | |
| | pyrazol-5-yl)pyrimidin-4-amine | |
| 215 | N-(3-(2,4-dimethoxyphenyl)- | — |
| | 1H-pyrazol-5-yl)-2-((2R,6S)- | |
| | 2,6- | |
| | dimethylmorpholino)pyrimidin- | |
| | 4-amine | |
| 216 | N-(3-(3,5-dimethoxyphenyl)- | — |
| | 1H-pyrazol-5-yl)-2-((2R,6S)- | |
| | 2,6- | |
| | dimethylmorpholino)pyrimidin- | |
| | 4-amine | |
| 217 | 2-((2R,6S)-2,6- | LCMS: 404.30 [M + 1]⁺ |
| | dimethylmorpholino)-N-(3-(1- | |
| | methyl-1H-indol-5-yl)-1H- | |
| | pyrazol-5-yl)pyrimidin-4-amine | |
| 218 | N-(3-(2,5-difluoro-4- | [M + H]⁺ 417.1869/¹H NMR (400 MHz, |
| | methoxyphenyl)-1H-pyrazol-5- | DMSO-d₆): δ 1.17 (6H, d, J = 6.0 Hz); |
| | yl)-2-((2R,6S)-2,6- | 2.46-2.52 (2H, m); 3.54-3.59 (2H, m); 3.90 |
| | dimethylmorpholino)pyrimidin- | (3H, s); 4.48-4.51 (2H, m); 6.26 (1H, s); |
| | 4-amine | 6.97 (1H, s); 7.27 (1H, dd, J = 12.4; 7.2 |
| | | Hz); 7.72 (1H, dd, J = 12.4; 7.2 Hz); 7.92 |
| | | (1H, d, J = 4.8 Hz); 9.68 (1H, s); 12.65 |
| | | (1H, s). ¹³C NMR (100 MHz, DMSO-d₆): δ |
| | | 19.21; 49.66; 57.14; 71.47; 96.81; 97.02; |
| | | 103.56 (t, ²J_{C-F} = 28.5 Hz); 109.44 (t, |
| | | ²J_{C-F} = 21.5 Hz); 113.45 (t, ²J_{C-F} = 22.1 Hz); |
| | | 135.15; 148.11; 148.37 (t, ¹J_{C-F} = 238.0 |
| | | Hz); 149.78; 155.07 (t, ¹J_{C-F} = 242.0 Hz); |
| | | 156.41; 160.02; 161.50. |
| 219 | N-(3-(4-(difluoromethoxy)-2- | LCMS: 435.20 [M + 1]⁺; ¹H NMR (400 |
| | fluorophenyl)-1H-pyrazol-5- | MHz, CD₃CN-d₃) δ = 8.04-7.93 (m, 2H). |
| | yl)-2-((2R,6S)-2,6- | 7.87-7.78 (m, 1H), 7.15-7.08 (m, 2H). |
| | dimethylmorpholino)pyrimidin- | 7.06-6.67 (m, 2H), 6.23 (d, J = 5.6 Hz, |
| | 4-amine | 1H), 4.53 (dd, J = 1.6, 13.1 Hz, 2H), 3.71- |
| | | 3.57 (m, 2H), 2.56 (dd, J = 10.7, 13.1 Hz, |
| | | 2H), 1.22 (d, J = 6.1 Hz, 6H) |
| 220a | N-(3-(4- | — |
| | (difluoromethoxy)phenyl)-1H- | |
| | pyrazol-5-yl)-2-(2,6- | |
| | dimethylmorpholino)pyrimidin- | |
| | 4-amine | |
| 220b | N-(3-(4- | — |
| | (difluoromethoxy)phenyl)-1H- | |
| | pyrazol-5-yl)-2-((2R,6S)-2,6- | |
| | dimethylmorpholino)pyrimidin- | |
| | 4-amine | |
| 221 | N-(3-(4- | — |
| | (difluoromethoxy)phenyl)-1H- | |
| | pyrazol-5-yl)-2-(4- | |
| | methylpiperidin-1-yl)pyrimidin- | |
| | 4-amine | |
| 222 | N-(3-(4- | — |
| | (difluoromethoxy)phenyl)-1H- | |
| | pyrazol-5-yl)-2-(4- | |
| | fluoropiperidin-1-yl)pyrimidin- | |
| | 4-amine | |
| 223 | N-(3-(4- | — |
| | (difluoromethoxy)phenyl)-1H- | |
| | pyrazol-5-yl)-2-(4- | |
| | (trifluoromethyl)piperidin-1- | |
| | yl)pyrimidin-4-amine | |
| 224 | N-(3-(4- | — |
| | (difluoromethoxy)phenyl)-1H- | |
| | pyrazol-5-yl)-2-(2,2,6,6- | |
| | tetramethylmorpholino)pyrimidin- | |
| | 4-amine | |
| 225 | N-(3-(4- | — |
| | (difluoromethoxy)phenyl)-1H- | |
| | pyrazol-5-yl)-2-(3,3- | |
| | dimethylmorpholino)pyrimidin- | |
| | 4-amine | |
| 226 | N-(3-(4- | — |
| | (difluoromethoxy)phenyl)-1H- | |
| | pyrazol-5-yl)-2-(2,2- | |
| | dimethylmorpholino)pyrimidin- | |
| | 4-amine | |
| 227 | N-(3-(4- | LCMS: 417.10 [M + 1]⁺ |
| | (difluoromethoxy)phenyl)-1H- | |
| | pyrazol-5-yl)-2-(3,5- | |
| | dimethylmorpholino)pyrimidin- | |
| | 4-amine | |
| 228 | N-(3-(4- | — |
| | (difluoromethoxy)phenyl)-1H- | |
| | pyrazol-5-yl)-2-(2- | |
| | methylmorpholino)pyrimidin-4- | |
| | amine | |
| 229 | N-(3-(4- | [M + H]⁺ 403.1703/¹H NMR (400 MHz, |
| | (difluoromethoxy)phenyl)-1H- | DMSO-d₆): δ 1.35 (3H, d, J = 6.4 Hz); |
| | pyrazol-5-yl)-2-(3- | 3.44-3.56 (2H, m); 3.65-3.82 (2H, m); |
| | methylmorpholino)pyrimidin-4- | 4.00-4.04 (2H, m); 4.42-4.44 (1H, m); 6.46 |
| | amine | (1H, s); 6.93 (1H, s); 7.25 (1H, t, J = 73.8 |
| | | Hz); 7.28 (2H, d, J = 8.6 Hz); 7.77 (2H, d, |
| | | J = 8.6 Hz); 7.89-7.91 (1H, m). |
| 230 | N-(3-(4- | — |
| | (difluoromethoxy)phenyl)-1H- | |
| | pyrazol-5-yl)-2-(2-(trifluoro- | |
| | methyl)morpholino)pyrimidin- | |
| | 4-amine | |
| 231 | N-(3-(4- | — |
| | (difluoromethoxy)phenyl)-1H- | |
| | pyrazol-5-yl)-2-(tetrahydro-1H- | |
| | furo[3,4-c]pyrrol-5(3H)- | |
| | yl)pyrimidin-4-amine | |
| 232 | 2-(3-azabicyclo[3.1.0]hexan-3- | — |
| | yl)-N-(3-(4- | |
| | (difluoromethoxy)phenyl)-1H- | |
| | pyrazol-5-yl)pyrimidin-4-amine | |
| 233 | N-(3-(4- | — |
| | (difluoromethoxy)phenyl)-1H- | |
| | pyrazol-5-yl)-2-(7- | |
| | azaspiro[3.5]nonan-7- | |
| | yl)pyrimidin-4-amine | |
| 234 | N-(3-(4- | — |
| | (difluoromethoxy)phenyl)-1H- | |
| | pyrazol-5-yl)-2-(4,4- | |
| | dimethylpiperidin-1- | |
| | yl)pyrimidin-4-amine | |
| 235 | N-(3-(4- | — |
| | (difluoromethoxy)phenyl)-1H- | |
| | pyrazol-5-yl)-2-(6- | |
| | azaspiro[2.5]octan-6- | |
| | yl)pyrimidin-4-amine | |
| 236 | N-(3-(4- | — |
| | (difluoromethoxy)phenyl)-1H- | |
| | pyrazol-5-yl)-2-(3-methyl-8- | |
| | azabicyclo[3.2.1]octan-8- | |
| | yl)pyrimidin-4-amine | |
| 237 | N-(3-(4- | — |
| | (difluoromethoxy)phenyl)-1H- | |
| | pyrazol-5-yl)-2-(2-oxa-6- | |
| | azaspiro[3.3]heptan-6- | |
| | yl)pyrimidin-4-amine | |
| 238 | N-(3-(4- | — |
| | (difluoromethoxy)phenyl)-1H- | |
| | pyrazol-5-yl)-2-(3-methyl-8- | |
| | azabicyclo[3.2.1]octan-8- | |
| | yl)pyrimidin-4-amine | |
| 239 | N-(3-(4- | — |
| | (difluoromethoxy)phenyl)-1H- | |
| | pyrazol-5-yl)-2-(4- | |
| | methylpiperazin-1- | |
| | yl)pyrimidin-4-amine | |
| 240 | N-(3-(4- | [M + H]⁺ 417.1865/¹H NMR (500 MHz, |
| | (difluoromethoxy)phenyl)-1H- | DMSO-d₆): δ 1.16 (6H, d, J = 6.4 Hz); |
| | pyrazol-5-yl)-2-((2R,6R)-2,6- | 3.47-3.48 (2H, m); 3.82-3.85 (2H, m); |
| | dimethylmorpholino)pyrimidin- | 4.11-4.12 (2H, m); 6.43 (1H, d, J = 6.5 |
| | 4-amine | Hz); 6.92 (1H, s); 7.15 (1H, t, J = 73.9 Hz); |
| | | 7.24 (2H, d, J = 7.7 Hz); 7.74 (2H, d, J = |
| | | 7.7 Hz); 7.81-7.83 (1H, m). |
| 241 | N-(3-(4- | [M + H]⁺ 417.1842/¹H NMR (500 MHz, |
| | (difluoromethoxy)phenyl)-1H- | DMSO-d₆): δ 1.17 (6H, d, J = 6.4 Hz); |
| | pyrazol-5-yl)-2-((25,6S)-2,6- | 3.48-3.49 (2H, m); 3.83-3.85 (2H, m); |
| | dimethylmorpholino)pyrimidin- | 4.12-4.14 (2H, m); 6.44 (1H, d, J = 5.9 |
| | 4-amine | Hz); 6.94 (1H, s); 7.21 (1H, t, J = 73.8 Hz); |
| | | 7.27 (2H, d, J = 8.4 Hz); 7.75-7.86 (3H, m). |
| 242 | N-(3-(4- | LCMS: 373.10 [M + 1]⁺ |
| | (difluoromethoxy)phenyl)-1H- | |
| | pyrazol-5-yl)-2-(pyrrolidin-1- | |
| | yl)pyrimidin-4-amine | |
| 243 | N-(3-(4- | LCMS: 387.20 [M + 1]⁺ |
| | (difluoromethoxy)phenyl)-1H- | |
| | pyrazol-5-yl)-2-(piperidin-1- | |
| | yl)pyrimidin-4-amine | |
| 244 | 2-(azetidin-1-yl)-N-(3-(4- | LCMS: 359.10 [M + 1]⁺ |
| | (difluoromethoxy)phenyl)-1H- | |
| | pyrazol-5-yl)pyrimidin-4-amine | |
| 245 | N-(3-(4- | LCMS: 373.05 [M + 1]⁺ |
| | (difluoromethoxy)phenyl)-1H- | |
| | pyrazol-5-yl)-2-(3- | |
| | methylazetidin-1-yl)pyrimidin- | |
| | 4-amine | |
| 246 | N-(3-(4- | LCMS: 377.00 [M + 1]⁺ |
| | (difluoromethoxy)phenyl)-1H- | |
| | pyrazol-5-yl)-2-(3- | |
| | fluoroazetidin-1-yl)pyrimidin- | |
| | 4-amine | |
| 247 | N-(3-(4- | LCMS: 387.05 [M + 1]⁺ |
| | (difluoromethoxy)phenyl)-1H- | |
| | pyrazol-5-yl)-2-(3,3- | |
| | dimethylazetidin-1- | |
| | yl)pyrimidin-4-amine | |
| 248 | N-(3-(4- | LCMS: 391.20 [M + 1]⁺ |
| | (difluoromethoxy)phenyl)-1H- | |
| | pyrazol-5-yl)-2-(3-fluoro-3- | |
| | methylazetidin-1-yl)pyrimidin- | |
| | 4-amine | |
| 249 | 2-(3,3-difluoroazetidin-1-yl)-N- | LCMS: 395.20 [M + 1]⁺ |
| | (3-(4- | |
| | (difluoromethoxy)phenyl)-1H- | |
| | pyrazol-5-yl)pyrimidin-4-amine | |
| 250 | N-(3-(4- | — |
| | (difluoromethoxy)phenyl)-1H- | |
| | pyrazol-5-yl)-2-(2- | |
| | methylpyrrolidin-1- | |
| | yl)pyrimidin-4-amine | |
| 251 | N-(3-(4- | — |
| | (difluoromethoxy)phenyl)-1H- | |
| | pyrazol-5-yl)-2-(3- | |
| | methylpyrrolidin-1- | |
| | yl)pyrimidin-4-amine | |
| 252 | N-(3-(4- | LCMS: 409.00 [M + 1]⁺ |
| | (difluoromethoxy)phenyl)-1H- | |
| | pyrazol-5-yl)-2-(3,3- | |
| | difluoropyrrolidin-1- | |
| | yl)pyrimidin-4-amine | |
| 253 | N-(3-(4- | LCMS: 423.10 [M + 1]⁺ |
| | (difluoromethoxy)phenyl)-1H- | |
| | pyrazol-5-yl)-2-(4,4- | |
| | difluoropiperidin-1- | |
| | yl)pyrimidin-4-amine | |
| 254 | 2-(2-oxa-5- | — |
| | azabicyclo[4.1.0]heptan-5-yl)- | |
| | N-(3-(4- | |
| | (difluoromethoxy)phenyl)-1H- | |
| | pyrazol-5-yl)pyrimidin-4-amine | |
| 255 | N-(3-(4- | [M + H]⁺ 403.1683/¹H NMR (400 MHz, |
| | (difluoromethoxy)phenyl)-1H- | DMSO-d₆): δ 1.95-1.96 (2H, m); 3.60-3.84 |
| | pyrazol-5-yl)-2-(1,4-oxazepan- | (10H, m); 6.45 (1H, d, J = 6.8 Hz); 6.92 |
| | 4-yl)pyrimidin-4-amine | (1H, s); 7.21 (1H, t, J = 74.0 Hz); 7.27 |
| | | (2H, d, J = 7.8 Hz); 7.74 (2H, d, J = 7.8 |
| | | Hz); 7.84 (1H, d, J = 6.8 Hz); 11.27 (1H, s). |
| 256 | (R)-N-(3-(4- | — |
| | (difluoromethoxy)phenyl)-1H- | |
| | pyrazol-5-yl)-2-(2- | |
| | methylmorpholino)pyrimidin-4- | |
| | amine | |
| 257 | (S)-N-(3-(4- | — |
| | (difluoromethoxy)phenyl)-1H- | |
| | pyrazol-5-yl)-2-(2- | |
| | methylmorpholino)pyrimidin-4- | |
| | amine | |
| 258 | 2-(azepan-1-yl)-N-(3-(4- | — |
| | (difluoromethoxy)phenyl)-1H- | |
| | pyrazol-5-yl)pyrimidin-4-amine | |
| 259 | 2-(3-oxa-8- | [M + H]⁺/¹H NMR (400 MHz, DMSO-d₆): |
| | azabicyclo[3.2.1]octan-8-yl)-N- | δ 1.99-2.10 (4H, m); 3.68-3.75 (4H, m); |
| | (3-(4- | 4.64-4.66 (2H, m); 6.42 (1H, d, J = 7.1 |
| | (difluoromethoxy)phenyl)-1H- | Hz); 6.88 (1H, s); 7.20 (1H, t, J = 73.8 Hz); |
| | pyrazol-5-yl)pyrimidin-4-amine | 7.26 (2H, d, J = 8.4 Hz); 7.75 (2H, d, J = |
| | | 8.4 Hz); 7.87 (1H, d, J = 7.1 Hz). |
| 260 | 2-(8-oxa-3- | [M + H]⁺ 415.1708/¹H NMR (400 MHz, |
| | azabicyclo[3.2.1]octan-3-yl)-N- | CDCl₃): δ 1.82-1.84 (2H, m); 1.95-1.98 |
| | (3-(4- | (2H, m); 3.25-3.39 (2H, m); 4.14-4.17 (2H, |
| | (difluoromethoxy)phenyl)-1H- | m); 4.48-4.50 (2H, m); 6.11 (1H, d, J = 5.8 |
| | pyrazol-5-yl)pyrimidin-4-amine | Hz); 6.28 (1H, s); 6.55 (1H, t, J = 73.6 Hz); |
| | | 7.02 (1H, s); 7.19 (2H, d, J = 8.4 Hz); 7.69 |
| | | (2H, d, J = 8.4 Hz); 8.06 (1H, d, J = 5.8 |
| | | Hz); |
| 261 | 2-((1S,4S)-2-oxa-5- | [M + H]⁺ 401.1550/¹H NMR (500 MHz, |
| | azabicyclo[2.2.1]heptan-5-yl)- | DMSO-d₆): δ 1.99-2.04 (2H, m); 3.46-3.88 |
| | N-(3-(4- | (4H, m); 4.79-5.02 (2H, m); 6.42 (1H, d, |
| | (difluoromethoxy)phenyl)-1H- | J = 7.0 Hz); 6.97-7.01 (1H, m); 7.22 (1H, t, |
| | pyrazol-5-yl)pyrimidin-4-amine | J = 73.8 Hz); 7.27 (2H, d, J = 8.6 Hz); 7.79 |
| | | (2H, d, J = 8.6 Hz); 7.84-7.86 (1H, m). |
| 262 | 2-((1R,4R)-2-oxa-5- | [M + H]⁺ 401.1542/¹H NMR (500 MHz, |
| | azabicyclo[2.2.1]heptan-5-yl)- | DMSO-d₆): δ 2.00-2.02 (2H, m); 3.46-3.87 |
| | N-(3-(4- | (4H, m); 4.78-5.02 (2H, m); 6.42 (1H, d, |
| | (difluoromethoxy)phenyl)-1H- | J = 7.0 Hz); 7.02-7.35 (4H, m); 7.78-7.83 |
| | pyrazol-5-yl)pyrimidin-4-amine | (3H, m). |
| 263 | (S)-N-(3-(4- | LCMS: 403.30 [M + 1]⁺ |
| | (difluoromethoxy)phenyl)-1H- | |
| | pyrazol-5-yl)-2-(3- | |
| | methylmorpholino)pyrimidin-4- | |
| | amine | |
| 264 | (R)-N-(3-(4- | LCMS: 403.25 [M + 1]⁺ |
| | (difluoromethoxy)phenyl)-1H- | |
| | pyrazol-5-yl)-2-(3- | |
| | methylmorpholino)pyrimidin-4- | |
| | amine | |
| 265 | N-(3-(4- | LCMS: 373.15 [M + 1]⁺ |
| | (difluoromethoxy)phenyl)-1H- | |
| | pyrazol-5-yl)-2-(2,2- | |
| | dimethylaziridin-1- | |
| | yl)pyrimidin-4-amine | |
| 274 | 2-((2R,6S)-2,6- | — |
| | dimethylmorpholino)-N-(3-(3- | |
| | fluoro-4-methoxyphenyl)-1H- | |
| | pyrazol-5-yl)-N- | |
| | methylpyrimidin-4-amine | |
| 275 | 2-((2R,6S)-2,6- | LCMS: 413.35 [M + 1]⁺ |
| | dimethylmorpholino)-N-(3-(2- | |
| | fluoro-4-methoxyphenyl)-1H- | |
| | pyrazol-5-yl)-N- | |
| | methylpyrimidin-4-amine | |
| 276 | N-(3-(4- | — |
| | (difluoromethoxy)phenyl)-1H- | |
| | pyrazol-5-yl)-2-((2R,6S)-2,6- | |
| | dimethylmorpholino)-N- | |
| | methylpyrimidin-4-amine | |
| 277 | 2-((2R,6S)-2,6- | LCMS: 413.35 [M + 1]⁺ |
| | dimethylmorpholino)-N-(3-(4- | |
| | (fluoromethoxy)phenyl)-1H- | |
| | pyrazol-5-yl)-N- | |
| | methylpyrimidin-4-amine | |
| 281 | 5-chloro-2-((2R,6S)-2,6- | [M + H]⁺ 433.1535/¹H NMR (500 MHz, |
| | dimethylmorpholino)-N-(3-(2- | DMSO-d₆): δ 1.14 (6H, d, J = 6.2 Hz); |
| | fluoro-4-methoxyphenyl)-1H- | 2.50-2.52 (2H, m); 3.54-3.58 (2H, m); 3.82 |
| | pyrazol-5-yl)pyrimidin-4-amine | (3H, s); 4.40-4.42 (2H, m); 6.78-6.99 (3H, |
| | | m); 7.73-8.04 (2H,m); 8.92 (1H, s), 12.85 |
| | | (1H, s). ¹³C NMR (100 MHz, DMSO-d₆): δ |
| | | 19.11; 49.80; 56.31; 71.36; 97.86 (d, |
| | | ³J_{C-F} = 10.1 Hz); 102.68 (d, ²J_{C-F} = 25.8 Hz); |
| | | 110.20 (d, ²J_{C-F} = 12.5 Hz); 111.67; |
| | | 128.17; 136.20; 148.13; 154.78; 155.23; |
| | | 159.65 (d, ¹J_{C-F} = 245.6 Hz); 159.67; |
| | | 160.97. |
| 282 | 2-((2R,6S)-2,6- | [M + H]⁺ 417.1850/¹H NMR (500 MHz, |
| | dimethylmorpholino)-5-fluoro- | DMSO-d₆): δ 1.15 (6H, d, J = 6.2 Hz); |
| | N-(3-(2-fluoro-4- | 2.47-2.50 (2H, m); 3.54-3.59 (2H, m); 3.82 |
| | methoxyphenyl)-1H-pyrazol-5- | (3H, s); 4.36-4.39 (2H, m); 6.71-7.03 (3H, |
| | yl)pyrimidin-4-amine | m); 7.73-7.77 (1H, m); 7.97 (1H, d, J = 2.5 |
| | | Hz); 9.79 (1H, s); 12.78 (1H, s). ¹³C NMR |
| | | (100 MHz, DMSO-d₆): δ 19.16; 50.20; |
| | | 56.31; 71.38; 97.28; 102.68 (d, ²J_{C-F} = 25.5 |
| | | Hz); 110.25 (d, ²J_{C-F} = 13.2 Hz); 111.69; |
| | | 128.12; 136.07; 140.03 (d, ¹J_{C-F} = 244.4 |
| | | Hz); 140.80 (d, ²J_{C-F} = 19.3 Hz); 148.40; |
| | | 149.29 (d, ³J_{C-F} = 11.0 Hz); 157.77; 159.62 |
| | | (d, ¹J_{C-F} = 241.0 Hz). |
| 283 | 2-((2R,6S)-2,6- | ¹H NMR (500 MHz, DMSO-d₆): δ 1.15 |
| | dimethylmorpholino)-N-(3-(2- | (3H, d, J = 6.2 Hz); 1.18 (3H, d, J = 7.1 |
| | fluoro-4-methoxyphenyl)-1H- | Hz); 2.05 (3H, s); 2.43-2.48 (2H, m); 3.54- |
| | pyrazol-5-yl)-5- | 3.58 (2H, m); 3.82 (3H, s); 4.41-4.43 (2H, |
| | methylpyrimidin-4-amine | m); 6.90-7.05 (3H, m); 7.76-7.77 (2H, m); |
| | | 8.73 (1H, s); 12.70 (1H, s). ¹³C NMR (100 |
| | | MHz, DMSO-d₆): δ 13.73; 19.20; 49.98; |
| | | 56.28; 71.43; 97.66 (d, ³J_{C-F} = 9.9 Hz); |
| | | 102.65 (d, ²J_{C-F} = 26.3 Hz); 104.12; 110.41 |
| | | (d, ²J_{C-F} = 12.6 Hz); 111.64; 128.03; |
| | | 135.83; 149.47; 155.87; 158.62; 159.61 (d, |
| | | ¹J_{C-F} = 247.0 Hz); 160.64; 160.71. |
| 286 | N-(3-(4- | LCMS: 435.10 [M + 1]⁺; |
| | (difluoromethoxy)phenyl)-1H- | |
| | pyrazol-5-yl)-2-(3,5- | |
| | dimethylmorpholino)-5- | |
| | fluoropyrimidin-4-amine | |
| 287 | N-(3-(4- | LCMS: 435.10 [M + 1]⁺; |
| | (difluoromethoxy)phenyl)-1H- | |
| | pyrazol-5-yl)-2-((2R,6S)-2,6- | |
| | dimethylmorpholino)-5- | |
| | fluoropyrimidin-4-amine | |
| 288 | 5-chloro-N-(3-(4- | LCMS: 451.10 [M + 1]⁺; |
| | (difluoromethoxy)phenyl)-1H- | |
| | pyrazol-5-yl)-2-((2R,6S)-2,6- | |
| | dimethylmorpholino)pyrimidin- | |
| | 4-amine | |
| 289 | 2-(azetidin-1-yl)-N-(3-(4- | LCMS: 377.00 [M + 1]⁺ |
| | (difluoromethoxy)phenyl)-1H- | |
| | pyrazol-5-yl)-5- | |
| | fluoropyrimidin-4-amine | |
| 290 | N-(3-(4- | LCMS: 405.20 [M + 1]⁺ |
| | (difluoromethoxy)phenyl)-1H- | |
| | pyrazol-5-yl)-5-fluoro-2-(2- | |
| | methylpyrrolidin-1- | |
| | yl)pyrimidin-4-amine | |
| 291 | N-(3-(4- | LCMS: 421.10 [M + 1]⁺ |
| | (difluoromethoxy)phenyl)-1H- | |
| | pyrazol-5-yl)-5-fluoro-2-(3- | |
| | methylmorpholino)pyrimidin-4- | |
| | amine | |
| 292 | N-(3-(4- | LCMS: 435.20 [M + 1]⁺ |
| | (difluoromethoxy)phenyl)-1H- | |
| | pyrazol-5-yl)-2-((2R,6R)-2,6- | |
| | dimethylmorpholino)-5- | |
| | fluoropyrimidin-4-amine | |
| 293 | N-(3-(4- | LCMS: 435.20 [M + 1]⁺ |
| | (difluoromethoxy)phenyl)-1H- | |
| | pyrazol-5-yl)-2-((2S,6S)-2,6- | |
| | dimethylmorpholino)-5- | |
| | fluoropyrimidin-4-amine | |
| 294 | (S)-N-(3-(4- | LCMS: 421.10 [M + 1]⁺ |
| | (difluoromethoxy)phenyl)-1H- | |
| | pyrazol-5-yl)-5-fluoro-2-(3- | |
| | methylmorpholino)pyrimidin-4- | |
| | amine | |
| 295 | (R)-N-(3-(4- | LCMS: 421.25 [M + 1]⁺ |
| | (difluoromethoxy)phenyl)-1H- | |
| | pyrazol-5-yl)-5-fluoro-2-(3- | |
| | methylmorpholino)pyrimidin-4- | |
| | amine | |
| 298 | N-(3-(2-fluoro-4- | ¹H NMR (500 MHz, DMSO-d₆): δ 3.41- |
| | methoxyphenyl)-1H-pyrazol-5- | 3.43 (4H, m); 3.70-3.72 (4H, m); 3.82 (3H, |
| | yl)-6-morpholinopyridin-2- | s); 6.11 (1H, d, J = 7.8 Hz); 6.47 (1H, d, |
| | amine | J = 6.0 Hz); 6.81-6.99 (3H, m); 7.34 (1H, t, |
| | | J = 7.8 Hz); 7.70-7.73 (1H, m); 9.01 (1H, s); |
| | | 12.41 (1H, s). |
| 299 | 6-(2,6-dimethylmorpholino)-N- | — |
| | (3-(2-fluoro-4-methoxyphenyl)- | |
| | 1H-pyrazol-5-yl)pyrazin-2- | |
| | amine | |
| 300 | 6-((2R,6S)-2,6- | — |
| | dimethylmorpholino)-N-(3-(2- | |
| | fluoro-4-methoxyphenyl)-1H- | |
| | pyrazol-5-yl)pyrazin-2-amine | |
| 301 | 6-((2R,6S)-2,6- | — |
| | dimethylmorpholino)-N-(3-(2- | |
| | fluoro-4-methoxyphenyl)-1H- | |
| | pyrazol-5-yl)pyridin-2-amine | |
| 302 | 4-((2R,6S)-2,6- | — |
| | dimethylmorpholino)-N-(3-(2- | |
| | fluoro-4-methoxyphenyl)-1H- | |
| | pyrazol-5-yl)-1,3,5-triazin-2- | |
| | amine | |
| 303 | 6-((2R,6S)-2,6- | — |
| | dimethylmorpholino)-5-fluoro- | |
| | N-(3-(2-fluoro-4- | |
| | methoxyphenyl)-1H-pyrazol-5- | |
| | yl)pyridin-2-amine | |
| 304 | 2-((2R,6S)-2,6- | — |
| | dimethylmorpholino)-3-fluoro- | |
| | N-(3-(2-fluoro-4- | |
| | methoxyphenyl)-1H-pyrazol-5- | |
| | yl)pyridin-4-amine | |
| 308 | N-(3-(4- | ¹H NMR (500 MHz, DMSO-d₆): δ 1.16 |
| | (difluoromethoxy)phenyl)-1H- | (6H, d, J = 6.2 Hz); 2.31-2.36 (2H, m); |
| | pyrazol-5-yl)-2-((2R,6S)-2,6- | 3.61-3.64 (2H, m); 3.98-4.00 (2H, m); 6.29 |
| | dimethylmorpholino)pyridin-4- | (1H, s); 6.57 (1H, s); 6.88 (1H, s); 7.26 |
| | amine | (2H, d, J = 8.4 Hz); 7.30 (1H, t, J = 74.0 |
| | | Hz); 7.80-7.82 (2H, m); 8.90 (1H, s); 12.72 |
| | | (1H, s). |
| 309 | N-(3-(4- | LCMS: 434.15 [M + 1]⁺ |
| | (difluoromethoxy)phenyl)-1H- | |
| | pyrazol-5-yl)-2-((2R,6S)-2,6- | |
| | dimethylmorpholino)-3- | |
| | fluoropyridin-4-amine | |
| 310 | N-(3-(4- | [M + H]⁺ 418.1802/¹H NMR (500 MHz, |
| | (difluoromethoxy)phenyl)-1H- | CDCl₃): δ 1.31-1.33 (6H, m); 2.84-2.89 |
| | pyrazol-5-yl)-4-((2R,6S)-2,6- | (2H, m); 3.67-3.75 (2H, m); 4.71-4.87 (2H, |
| | dimethylmorpholino)-1,3,5- | m); 6.60 (1H, t, J = 73.2 Hz); 6.63 (1H, s); |
| | triazin-2-amine | 7.27 (2H, d, J = 8.6 Hz); 7.75 (2H, d, J = |
| | | 8.6 Hz); 8.43 (1H, s); 10.45 (1H, s). |
| 311 | N-(3-(4- | — |
| | (difluoromethoxy)phenyl)-1H- | |
| | pyrazol-5-yl)-6-((2R,6S)-2,6- | |
| | dimethylmorpholino)pyrazin-2- | |
| | amine | |
| 315 | N-(3-(4- | LCMS: 416.20 [M + 1]⁺ |
| | (difluoromethoxy)phenyl)-1H- | |
| | pyrazol-5-yl)-6-((2R,6S)-2,6- | |
| | dimethylmorpholino)pyridin-2- | |
| | amine | |
| 316 | N-(3-(4- | LCMS: 434.20 [M + 1]⁺ |
| | (difluoromethoxy)phenyl)-1H- | |
| | pyrazol-5-yl)-6-((2R,6S)-2,6- | |
| | dimethylmorpholino)-5- | |
| | fluoropyridin-2-amine | |
| 317 | 2-(3,6-dihydro-2H-pyran-4-yl)- | [M + H]⁺ 368, 1513/¹H NMR (400 MHz, |
| | N-(3-(2-fluoro-4- | DMSO-d₆): δ 2.56-2.58 (2H, m); 3.79-3.82 |
| | methoxyphenyl)-1H-pyrazol-5- | (5H, m); 4.28-4.29 (2H, m); 6.89-7.01 (3H, |
| | yl)pyrimidin-4-amine | m); 7.11 (1H, s); 7.73 (1H, t, J = 8.8 Hz); |
| | | 8.26 (1H, d, J = 5.6 Hz), 9.96 (1H, s); |
| | | 12.68 (1H, s). |
| 318 | N-(3-(2-fluoro-4- | [M + H]⁺ 370.1672/¹H NMR (400 MHz, |
| | methoxyphenyl)-1H-pyrazol-5- | DMSO-d₆): δ 1.80-1.90 (5H, m); 2.86-2.91 |
| | yl)-2-(tetrahydro-2H-pyran-4- | (1H, m); 3.32-3.48 (3H, m); 3.83 (3H, s), |
| | yl)pyrimidin-4-amine | 3.93-3.95 (2H, m); 6.91-7.02 (4H, m); 7.73 |
| | | (1H, t, J = 8.8 Hz); 8.23 (1H, d, J = 5.2 |
| | | Hz); 9.93 (1H, s); 12.68 (1H, s). |

### Example 2: Exemplary Biological Activity of Compounds of the Disclosure

### Cell Culture and Reagents

Human colon carcinoma cell line HCT-116 was a kind gift from Igor Ronninson from University of South Caroli, Columbia, USA. Human breast cancer cell line, JIMT-1 was kindly provided by Ali Osmay Güre from Bilkent University, Ankara, Turkey. Cells were cultured in Dulbecco's modified Eagle's medium (Lonza, NJ, USA), supplemented with 10% fetal bovine serum (FBS, Lonza), 1% non-essential amino acid (NEAA), 2 mM L-glutamine (Sigma Aldrich, MO, USA) and 50 U/ml penicillin/streptomycin (P/S). HCT-116 cells were cultured in McCoy's 5A (modified) (Gibco) medium supplemented with FBS, NEAA, L-glutamine and P/S. All cell lines were tested regularly using MycoAlert Mycoplasma Detection Kit (Lonza). The cumulative length of the cells between thawing and use in the experiments was less than 20 passages.

### Cell Viability Assays

Newly synthesized molecules were dissolved in 100% DMSO to yield a stock concentration of 10 mM. For cell viability assay, JIMT-1 (3×10³ cells/well) and HCT-116 (4×10³), cells were seeded into 96-well plates, and 24 hours after cell seeding inhibitor treatments were performed at different concentrations. Cell viability was measured 72 hours after treatment with Sulforhodamine B (SRB, Sigma Aldrich) assay as recommended by the manufacturer.

TABLE 3
Cell viability data in JIMT1 and HCT116

| Example | JIMT1 IC₅₀ | HCT116 IC₅₀ |
|---|---|---|
| 5 | 0.005 | 0.047 |
| 27 | 0.367 | 0.321 |
| 56 | 0.023 | 0.036 |
| 59b | 0.017 | 0.003 |
| 69 | 0.025 | 0.005 |
| 70 | 0.026 | 0.028 |
| 86b | 0.007 | 0.005 |
| 87 | 0.025 | 0.025 |
| 89 | 0.031 | 0.009 |
| 90 | 0.365 | 0.733 |
| 92 | 0.245 | 0.488 |
| 94 | 0.029 | 0.046 |
| 96 | 0.047 | 0.089 |
| 106 | 0.062 | 0.091 |
| 129b | 0.003 | 0.034 |
| 130 | 0.018 | 0.007 |
| 131 | 0.053 | 0.047 |
| 132 | 0.018 | 0.038 |
| 133 | 0.606 | 6.56 |
| 135 | 0.23 | 0.88 |
| 136 | 0.895 | 1.299 |
| 137 | 0.024 | 0.059 |
| 138 | 0.015 | 0.052 |
| 139 | 0.091 | 0.146 |
| 140 | 0.107 | 0.457 |
| 141 | 0.041 | 0.163 |
| 142 | 0.266 | 1.21 |
| 143 | 0.426 | 1.01 |
| 144 | 0.119 | 0.33 |
| 145 | 0.101 | 0.279 |
| 146 | 0.479 | 0.534 |
| 148 | 0.213 | 0.986 |
| 149 | 0.065 | 0.115 |
| 150 | 0.016 | 0.023 |
| 151 | 0.018 | 0.052 |
| 152 | 0.005 | 0.011 |
| 153 | 0.006 | 0.01 |
| 159 | 0.047 | 0.076 |
| 160 | 0.039 | 0.147 |
| 167 | 0.041 | 0.089 |
| 174 | 10 | NA |
| 177 | 10 | NA |
| 181 | 2.63 | 4.48 |
| 184 | 8.79 | 20.03 |
| 187 | 0.158 | 0.228 |
| 190 | 0.251 | 0.484 |
| 191b | 1.57 | 1.52 |
| 192b | 4.64 | 8.97 |
| 196 | 0.383 | 0.619 |
| 197 | NA | 0.255 |
| 198 | NA | 0.08 |
| 199 | NA | 0.09 |
| 200 | 0.143 | 0.386 |
| 201 | 0.076 | 0.091 |
| 202 | 0.187 | 0.722 |
| 203 | 1.7 | 2.49 |
| 205 | 0.027 | 0.096 |
| 206 | NA | 4.64 |
| 207 | 0.974 | 5.53 |
| 208 | 0.212 | 0.448 |
| 209 | NA | 0.108 |
| 210 | 0.03 | 0.031 |
| 211 | 0.043 | 0.039 |
| 217 | 0.006 | 0.005 |
| 218 | 0.013 | 0.020 |
| 227 | 1.433 | 2.257 |
| 229 | 0.096 | 0.062 |
| 240 | 0.103 | 1.058 |
| 241 | 0.046 | 1.81 |
| 242 | 0.11 | 1.487 |
| 243 | 0.017 | 0.053 |
| 244 | 0.046 | 1.12 |
| 245 | 0.047 | 10 |
| 246 | 0.071 | 10 |
| 247 | 0.142 | 10 |
| 248 | 1.558 | 10 |
| 249 | 0.305 | 10 |
| 252 | 0.122 | 1.26 |
| 253 | 0.148 | 1.56 |
| 255 | 0.279 | 0.312 |
| 259 | 0.065 | 10 |
| 260 | 0.648 | 8.98 |
| 261 | 0.4 | 7.2 |
| 262 | 0.329 | 10 |
| 263 | 0.03 | 0.079 |
| 264 | 0.127 | 0.302 |
| 275 | 0.013 | 0.046 |
| 277 | 0.204 | 0.269 |
| 281 | 0.131 | 0.436 |
| 282 | 0.045 | 0.046 |
| 283 | 0.212 | NA |
| 286 | 1.685 | 5.217 |
| 287 | 0.049 | 0.091 |
| 288 | 0.443 | 0.456 |
| 289 | 0.18 | 0.371 |
| 290 | 0.278 | 0.337 |
| 291 | 0.777 | 1.713 |
| 292 | 0.659 | 0.996 |
| 293 | 0.255 | 0.332 |
| 294 | 0.636 | 1.139 |
| 295 | 10 | 7.508 |
| 298 | 0.034 | NA |
| 308 | 0.053 | NA |
| 310 | 2.35 | 2.96 |
| 317 | 0.012 | 0.024 |
| 318 | 0.319 | 0.293 |

### Example 3: Further Exemplary Biological Activity of Compounds of the Disclosure

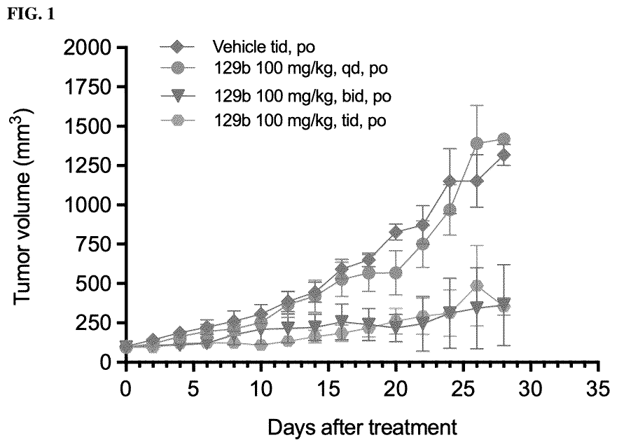

Six-to-eight-week-old female athymic nude mice were housed with a temperature-controlled and 12-hour light/12-hour dark cycle environment. For in vivo colon cancer tumor growth, 1×10⁶ RKO cells were prepared in 100 μL of DMEM and injected into right flank of female nude mice. Mouse weight and tumor volume were measured twice a week. Tumor volumes were calculated as length×width²×0.5. Once the tumor volume had reached about 100 mm³, xenografts were randomized into groups (5 mice per group). Animals were treated with vehicle and Compound 129b. The formulation for vehicle and Compound 129b was 50% PEG400 and 20% Cremophor RH40 final (50% of 40% Cremophor) in Acetate buffer, PH=4. The mouse were sacrificed after 4 weeks or if the tumors reached a predefined tumor volume cut-off of 2500 mm³. Compound 129b showed strong tumor growth inhibition in the RKO xenograft. For instance, at the highest does, a TGI of at least 80% was observed (FIG. 1). PGP-71

TABLE 4
Dosing Schedule for Exemplary in vivo Study

| Group | | Mice/ | Dose | Dose Vol | | | Of |
|---|---|---|---|---|---|---|---|
| # | Compound | group | (mg/kg) | (μl/g) | Route | Regimen | doses |
| 1 | Vehicle | 5 | — | 5 | PO | TID | 28 Days |
| 2 | 129b | 5 | 100 | 5 | PO | QD | 28 Days |
| 3 | 129b | 5 | 100 | 5 | PO | BID | 28 Days |
| 4 | 129b | 5 | 100 | 5 | PO | TID | 28 Days |

Compound 129b was also tested in CT-26 mouse colon xenograft models. For CT-26 xenografts 5×10⁵ CT-26 cells in 100 μL was injected in the right flank of six-to-eight-week-old female Balb/c mice. When the tumor volume reached mean of ˜100 mm³, mice were randomized in a group of 5 and treatment was initiated vehicle, 15 mpk QD, 25 mpk QD, 50 mpk QD, 5 mpk IV BIW, and 5 mpk IP QD of Compound 129b. Strong and statistically significant tumor growth inhibition or regression were seen in this model.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A compound represented by formula I or a pharmaceutically acceptable salt thereof: 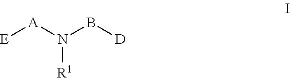
wherein,
E and B are each independently aryl, heteroaryl, or heterocyclyl;
D is heterocyclyl;
A is heteroaryl; and
R¹ is H, alkyl, or benzyl.

2. The compound of claim 1, wherein A is not isoxazole.

3. The compound of claim 1 or 2, wherein A is pyrrole, furan, selenophene, thiophene, imidazole, pyrazole, oxazole, oxathiole, isoxathiole, thiazole, isothiazole, triazole, furazan, oxadiazole, thiadiazole, dioxazole, or dithiazole.

4. The compound of any one of claims 1-3, wherein A is pyrazole.

5. The compound of any one of claims 1-4, wherein the compound is represented by formula Ia or Ib: 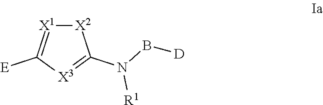 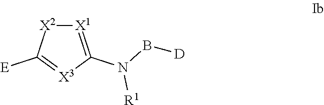
wherein,
E and B are each independently aryl, heteroaryl, or heterocyclyl;
D is heterocyclyl;
X¹ is selected from CH₂, NR², O, and S;
X² is selected from CH or N;
X³ is CR³ or N;
R¹ and R² are each independently H, alkyl, or benzyl; and
R³ is H, alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, or sulfonamide.

6. The compound of any one of claims 1-5, wherein the compound is represented by formula Ia: 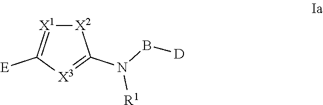
wherein,
E and B are each independently aryl, heteroaryl, or heterocyclyl;
D is heterocyclyl;
X¹ is C or N;
X² is CH₂, NR², O, or S;
X³ is CR³ or N;
R¹ and R² are each independently H, alkyl, or benzyl; and
R³ is alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, or sulfonamide.

7. The compound of claim 5 or 6, wherein X¹ is N.

8. The compound of claim 5 or 6, wherein X¹ is CH.

9. The compound of any one of claims 5-8, wherein X² is NR².

10. The compound of any one of claims 5-9, wherein R² is H.

11. The compound of any one of claims 5-9, wherein R² is alkyl (e.g., methyl).

12. The compound of any one of claims 5-11, wherein X² is S.

13. The compound of any one of claims 5-11, wherein X² is O.

14. The compound of any one of claims 5-13, wherein X³ is CR³.

15. The compound of any one of claims 5-14, wherein R³ is H.

16. The compound of any one of claims 5-14, wherein R³ is alkyl (e.g., methyl).

17. The compound of any one of claims 5-16, wherein X³ is N.

18. The compound of any one of claims 1-17, wherein R¹ is H.

19. The compound of any one of claims 1-17, wherein R¹ is alkyl (e.g., methyl or ethyl).

20. The compound any one of claims 1-19, wherein B is heteroaryl (e.g., pyridinyl, pyrimidinyl, pyrazinyl, or triazinyl).

21. The compound of any one of claims 1-20, wherein B is pyrimidinyl.

22. The compound of any one of claims 1-21, wherein B is substituted with at least one R⁴ and each R⁴ is independently selected from alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide.

23. The compound of any one of claims 1-22, wherein B is substituted with at least one R⁴ and each R⁴ is independently selected from alkyl (e.g., methyl) and halo (e.g., chloro or fluoro).

24. The compound of claim 22 or 23, wherein B is substituted with 1 or 2 R⁴.

25. The compound of any one of claims 1-24, wherein D is N-linked heterocyclyl (e.g., aziridinyl, azetidinyl, pyrrolidinyl, piperidinyl, azepanyl, piperazinyl, pyranyl, dihydropyranyl, morpholinyl, thiomorpholinyl, dioxidethiomorpholinyl, oxaazabicycloheptanyl, azabicyclooctanyl, oxaazabicyclooctanyl, hexahydrofuropyrrolyl, or azabicyclohexanyl).

26. The compound of any one of claims 1-25, wherein D is substituted with at least one R⁵ and each R⁵ is independently selected from H, deuterium, alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide; or D is substituted with at least two R⁵s and two of the R⁵s combine to complete a bicyclic heterocyclyl.

27. The compound of any one of claims 1-26, wherein D is substituted with at least one R⁵ and each R⁵ is independently selected from alkyl (e.g., methyl, fluoromethyl, difluoromethyl, or trifluoromethyl), halo (e.g., fluoro), cycloalkyl (e.g., cyclopropyl or cyclobutyl), or heterocyclyl (e.g., oxetanyl).

28. The compound of claim 26 or 27, wherein D is substituted with 1 or 2 R⁵.

29. The compound of claim 26 or 27, wherein D is substituted with 1 R⁵.

30. The compound of claim 26 or 27, wherein D is substituted with 2 R⁵s.

31. The compound of any one of claims 1-30, wherein E is aryl (e.g., phenyl, dihydrobenzofuranyl, benzodioxolyl, or dihydroindenyl).

32. The compound of any one of claims 1-31, wherein E is phenyl.

33. The compound of any one of claims 1-30, wherein E is heteroaryl (e.g., pyridinyl, pyrazinyl, indolyl, such as N-methyl indolyl, or benzofuranyl).

34. The compound of any one of claims 1-30, wherein E is heterocyclyl (e.g., pyrrolidinyl).

35. The compound of any one of claims 1-34, wherein E is substituted with at least one R⁶ and each R⁶ is independently selected from alkyl with alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide.

36. The compound of any one of claims 1-35, wherein E is substituted with at least one R⁶ and each R⁶ is independently selected from alkyl (e.g., deuteroalkyl, methyl, ethyl, butyl, isopropyl, difluoromethyl, trifluoromethyl, or difluoroethyl), alkyloxy (e.g., deuteroalkyloxy, methoxy, ethoxy, difluoromethyoxy, or trifluoromethyoxy), alkylthio (e.g., methylthio), amino (e.g., dimethylamino), halo (e.g., fluoro or chloro), cyano, heterocyclyl (e.g., azetidinyl), and hydroxyl.

37. The compound of claim 35 or 36, wherein E is substituted with 1 R⁶.

38. The compound of claim 35 or 36, wherein E is substituted with 2 R⁶.

39. The compound of claim 35 or 36, wherein E is substituted with 3 R⁶.

40. The compound of any one of claims 1-39, wherein the compound is represented by formula Ic or a pharmaceutically acceptable salt thereof: 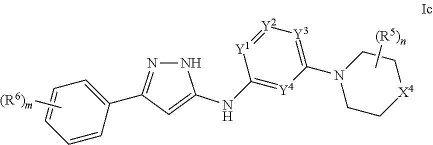
wherein,
Y¹ is N or CR^{8a};
Y² is N or CR^{8b};
Y³ is N or CR^{8c};
Y⁴ is N or CR^{8d};
X⁴ is CR^{5c}R^{5d}, O, or NR⁷;
R^{5c} and R^{5d} are each independently selected from deuterium, alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide; or R^{5c} and R^{5d} combine to form cycloalkyl;
R^{8a}, R^{8b}, R^{8c}, and R^{8d} are each independently selected from H, deuterium, alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide;
R⁷ is H, alkyl, acyl, acetyl, hydroxyl, alkoxy, cycloalkyl;
m is 1-5; and
n is 1-8.

41. The compound of claim 40, wherein Y¹ is N.

42. The compound of claim 40, wherein Y¹ is CR^{8a}.

43. The compound of claim 42, wherein R^{8a} is H, alkyl (e.g., methyl) or halo (e.g., fluoro or chloro).

44. The compound of any one of claims 40-43, wherein Y² is N.

45. The compound of any one of claims 40-43, wherein Y² is CR^{8b}.

46. The compound of claim 45, wherein R^{8b} is H, alkyl (e.g., methyl) or halo (e.g., fluoro or chloro).

47. The compound of any one of claims 40-46, wherein Y³ is N.

48. The compound of any one of claims 40-46, wherein Y³ is CR^{8c}.

49. The compound of claim 48, wherein R^{8c} is H, alkyl (e.g., methyl) or halo (e.g., fluoro or chloro).

50. The compound of any one of claims 40-49, wherein Y⁴ is N.

51. The compound of any one of claims 40-49, wherein Y⁴ is CR^{8d}.

52. The compound of claim 51, wherein R^{8d} is H, alkyl (e.g., methyl) or halo (e.g., fluoro or chloro).

53. The compound of any one of claims 1-40, wherein the compound is represented by formula II or a pharmaceutically acceptable salt thereof: 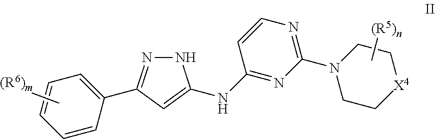
wherein,
X⁴ is CR^{5c}R^{5d}, O, or NR⁷;
R^{5c} and R^{5d} are each independently selected from deuterium, alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide; or R^{5c} and R^{5d} combine to form cycloalkyl;
R⁷ is H, alkyl, acyl, acetyl, hydroxyl, alkoxy, cycloalkyl;
m is 1-5; and
n is 1-8.

54. The compound of any one of claims 1-40, wherein the compound is represented by formula IIIa or a pharmaceutically acceptable salt thereof: 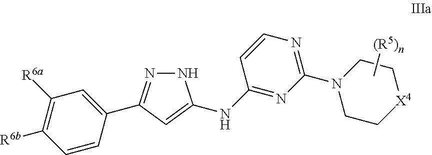
wherein,
R^{6a} and R^{6b} are each independently selected from H, deuterium, alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide.

55. The compound of any one of claims 1-40, wherein the compound is represented by formula IIIb or a pharmaceutically acceptable salt thereof: 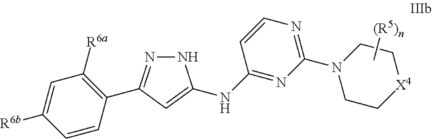
wherein,
R^{6a} and R^{6b} are each independently selected from H, deuterium, alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide.

56. The compound of any one of claims 53-56, wherein n is at least 2 and two or more instances of R⁵ combine to complete a bicyclic heterocyclyl (e.g., a oxaazabicycloheptanyl, azabicyclooctanyl, or oxaazabicyclooctanyl).

57. The compound of any one of claims 1-40, wherein the compound is represented by formula IVa or a pharmaceutically acceptable salt thereof: 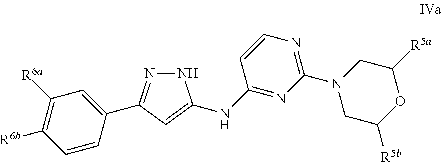
wherein,
R^{5a}, R^{5b}, R^{6a}, and R^{6b} are each independently selected from hydrogen, deuterium, alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide.

58. The compound of any one of claims 1-40, wherein the compound is represented by formula IVb or a pharmaceutically acceptable salt thereof: 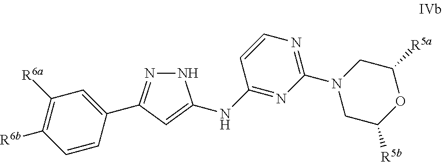
wherein,
R^{5a}, R^{5b}, R^{6a}, and R^{6b} are each independently selected from hydrogen, deuterium, alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide.

59. The compound of any one of claims 1-40, wherein the compound is represented by formula IVc or a pharmaceutically acceptable salt thereof: 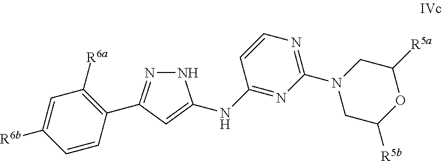
wherein,
R^{5a}, R^{5b}, R^{6a}, and R^{6b} are each independently selected from hydrogen, alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide.

60. The compound of any one of claims 1-40, wherein the compound is represented by formula IVd or a pharmaceutically acceptable salt thereof: 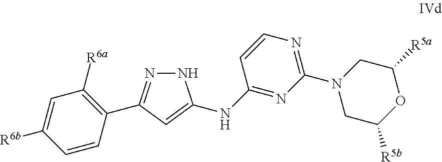
wherein,
R^{5a}, R^{5b}, R^{6a}, and R^{6b} are each independently selected from hydrogen, alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide.

61. The compound of any one of claims 1-40, wherein the compound is represented by formula IVe or a pharmaceutically acceptable salt thereof: 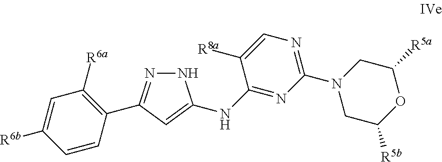
wherein,
R^{5a}, R^{5b}, R^{6a}, and R^{6b} are each independently selected from hydrogen, alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide; and
R^{8a} is selected from hydrogen, deuterium, alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide.

62. The compound of claim 61, wherein R^{8a} is halo (e.g., fluoro or chloro).

63. The compound of claim 61, wherein R^{8a} is alkyl (e.g., methyl).

64. The compound of any one of claims 1-40, wherein the compound is represented by formula IVf or a pharmaceutically acceptable salt thereof: 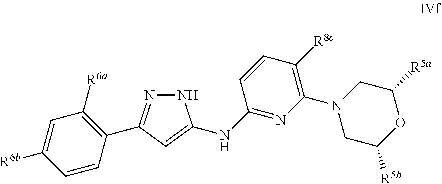
wherein,
R^{5a}, R^{5b}, R^{6a}, and R^{6b} are each independently selected from hydrogen, alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide; and
R^{8c} is selected from hydrogen, deuterium, alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide.

65. The compound of claim 64, wherein R^{8c} is halo (e.g., fluoro or chloro).

66. The compound of claim 64, wherein R^{8c} is alkyl (e.g., methyl).

67. The compound of any one of claims 1-40, wherein the compound is represented by formula IVg or a pharmaceutically acceptable salt thereof: 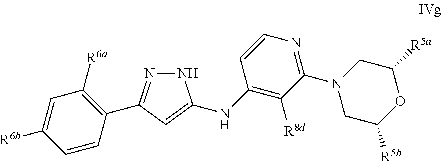
wherein,
R^{5a}, R^{5b}, R^{6a}, and R^{6b} are each independently selected from hydrogen, alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide; and
R^{8d} is selected from H, deuterium, alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide.

68. The compound of claim 67, wherein R^{8d} is halo (e.g., fluoro or chloro).

69. The compound of claim 67, wherein R^{8d} is alkyl (e.g., methyl).

70. The compound of any one of claims 1-40, wherein the compound is represented by formula IVh or a pharmaceutically acceptable salt thereof: 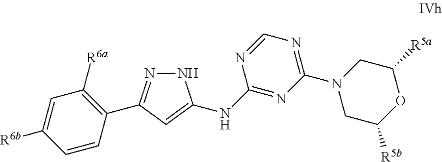
wherein,
R^{5a}, R^{5b}, R^{6a}, and R^{6b} are each independently selected from hydrogen, alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide.

71. The compound of any one of claims 57-70, wherein R^{5a} is alkyl (e.g., methyl).

72. The compound of any one of claims 57-71, wherein R^{5b} is alkyl (e.g., methyl).

73. The compound of any one of claims 1-40, wherein the compound is represented by formula Va or a pharmaceutically acceptable salt thereof: 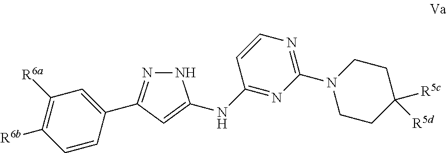
wherein,
R^{5c} and R^{5d} are each independently selected from alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide; or R^{5c} and R^{5d} combine to form cycloalkyl; and
R^{6a} and R^{6b} are each independently selected from alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide.

74. The compound of any one of claims 1-40, wherein the compound is represented by formula Vb or a pharmaceutically acceptable salt thereof: 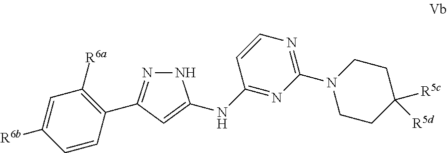
wherein,
R^{5c} and R^{5d} are each independently selected from alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide; or R^{5c} and R^{5d} combine to form cycloalkyl; and
R^{6a} and R^{6b} are each independently selected from alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide.

75. The compound of any one of claims 1-39, wherein the compound is represented by formula VIa or a pharmaceutically acceptable salt thereof: 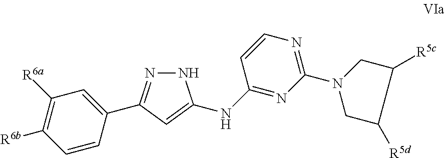
wherein,
R^{5c} and R^{5d} are each independently selected from alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide; or R^{5c} and R^{5d} combine to form cycloalkyl; and
R^{6a} and R^{6b} are each independently selected from alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide.

76. The compound of any one of claims 1-39, wherein the compound is represented by formula VIb or a pharmaceutically acceptable salt thereof: 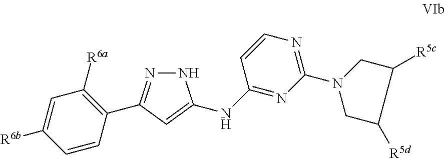
wherein,
R^{5c} and R^{5d} are each independently selected from alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide; or R^{5c} and R^{5d} combine to form cycloaklyl; and
R^{6a} and R^{6b} are each independently selected from alkyl, alkenyl, alkynyl, halo, hydroxyl, carboxyl, acyl, acetyl, ester, thioester, alkoxy, phosphoryl, amino, amide, cyano, nitro, azido, alkylthio, alkenyl, alkynyl, cycloalkyl, alkylsulfonyl, and sulfonamide.

77. The compound of any one of claims 54-76, wherein R^{6a} is halo (e.g., fluoro, chloro, or bromo).

78. The compound of any one of claims 54-77, wherein R^{6b} is alkoxy (e.g., deuteroalkoxy, methoxy, or fluoromethyoxy, such as monofluoromethyoxy or difluoromethyoxy).

79. The compound of any one of claims 54-78, wherein R^{6b} is alkyl (e.g., methyl, ethyl, fluoroalkyl, such as monofluoromethyl, difluoromethyl, or difluoroethyl).

80. The compound of any one of claims 54-77, wherein R^{6b} is alkylsulfonyl (e.g., methylsulfonyl).

81. The compound of any one of claims 72-80, wherein R^{5c} is alkyl (e.g., methyl or trifluoromethyl).

82. The compound of any one of claims 73-80, wherein R^{5c} is halo (e.g., fluoro).

83. The compound of any one of claims 73-82, wherein R^{5d} is hydrogen.

84. The compound of any one of claims 73-80, wherein R^{5c} and R^{5d} combine to form cycloalkyl (e.g., cyclopropyl or cyclobutyl).

85. The compound of claim 1, wherein the compound is selected from: 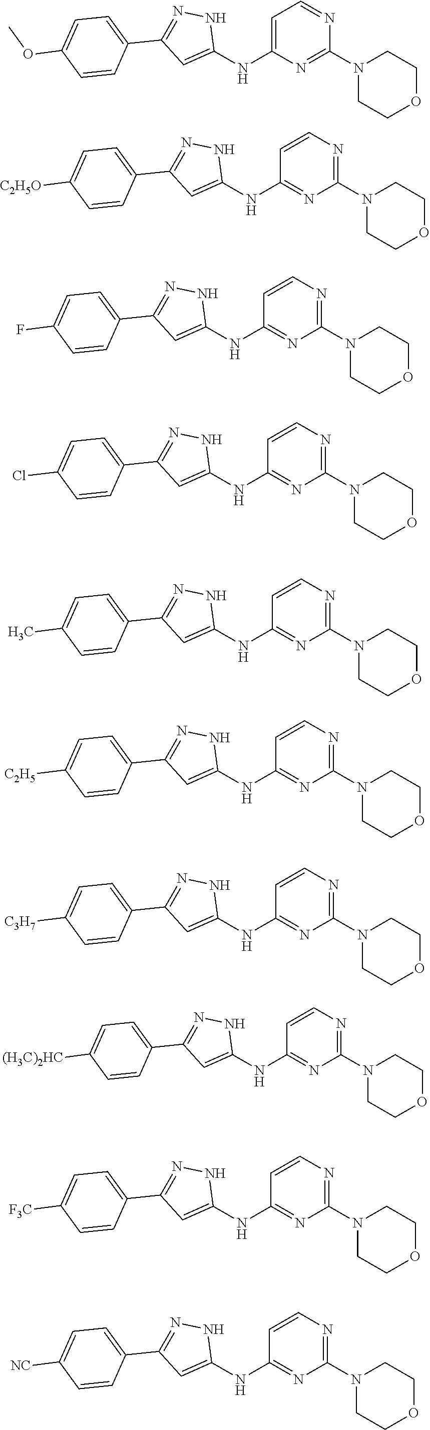 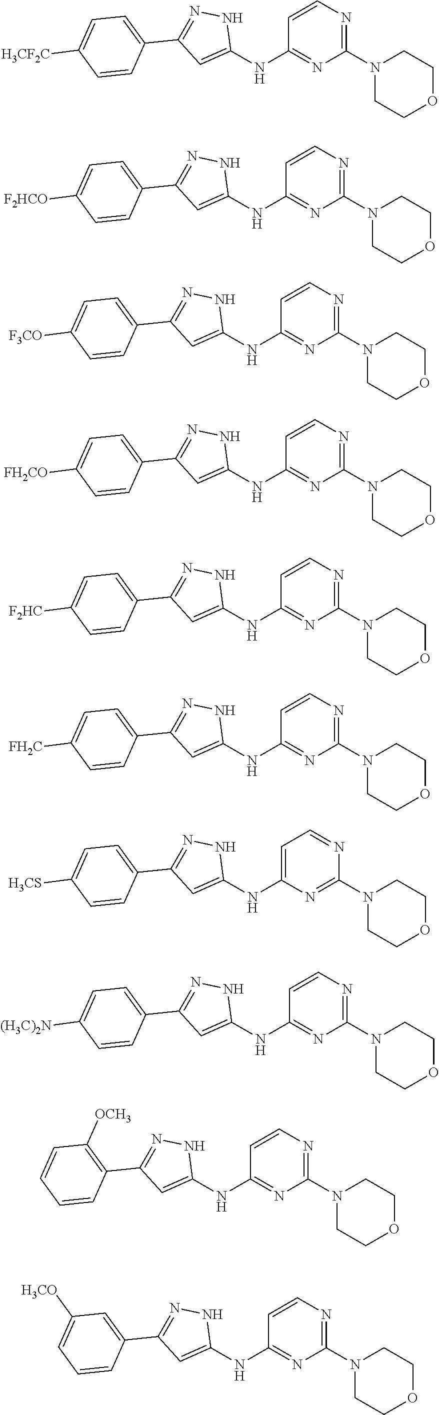 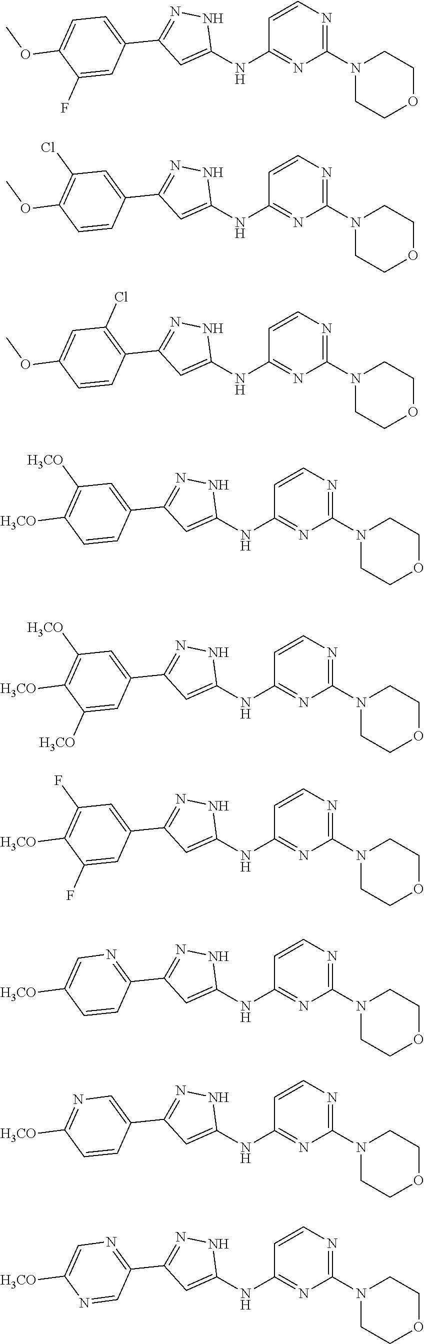 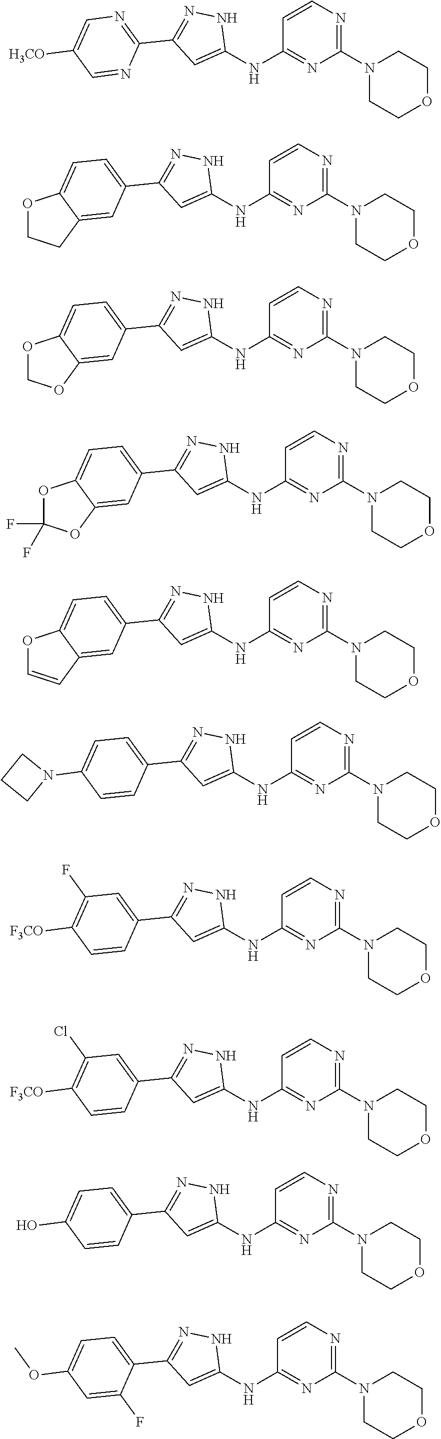 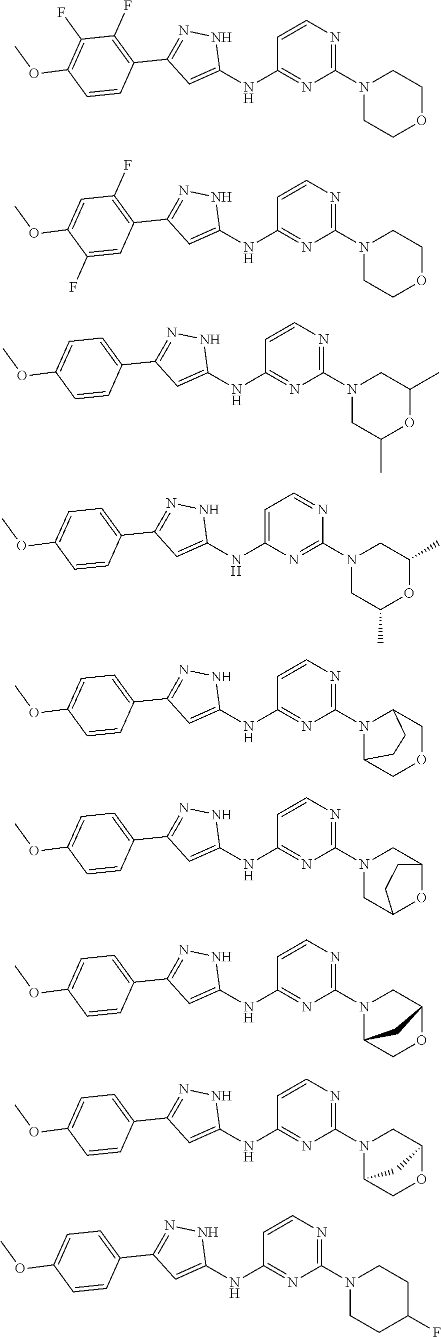 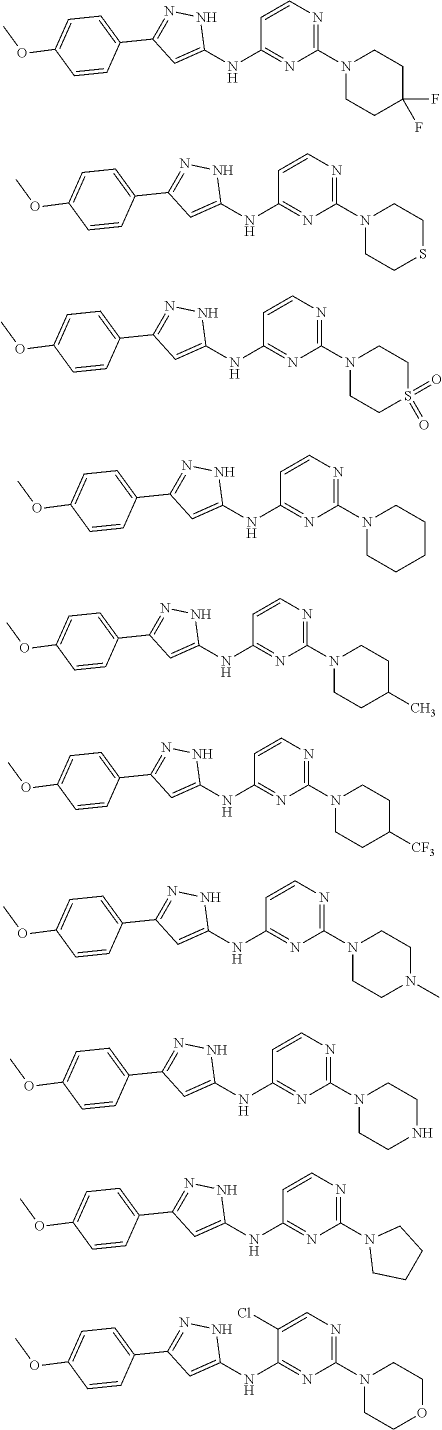 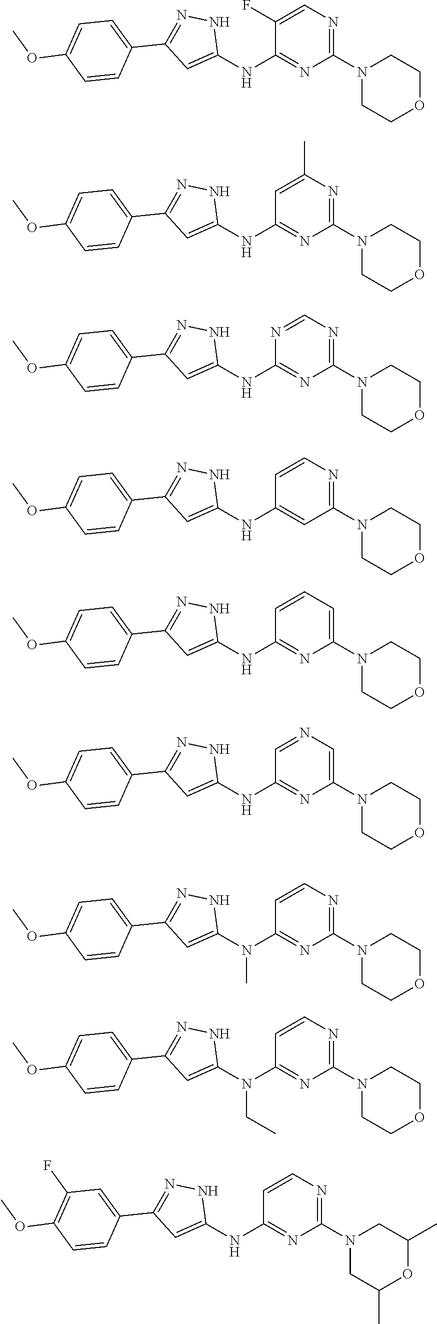 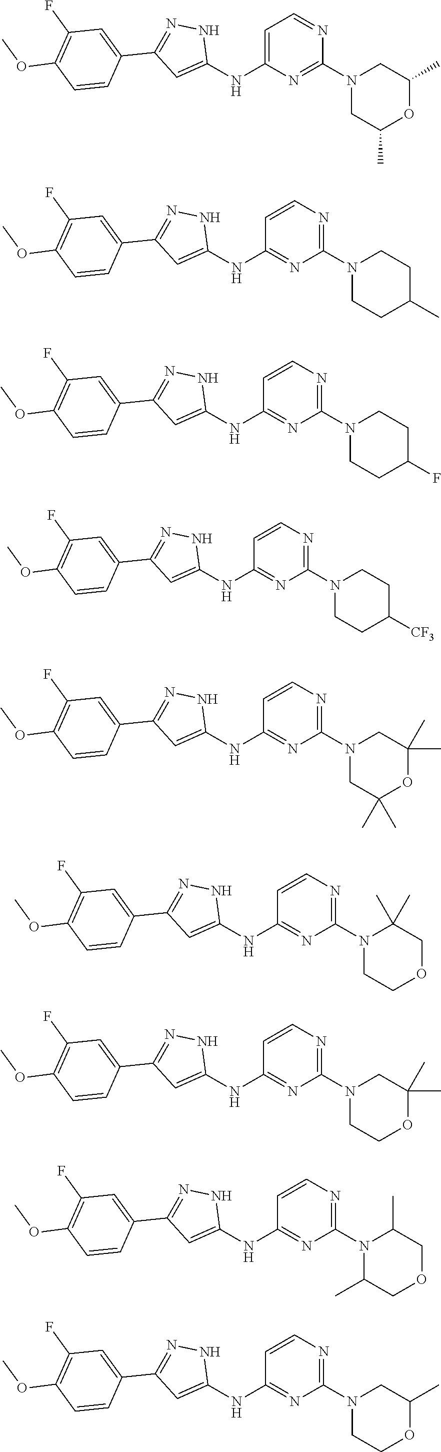 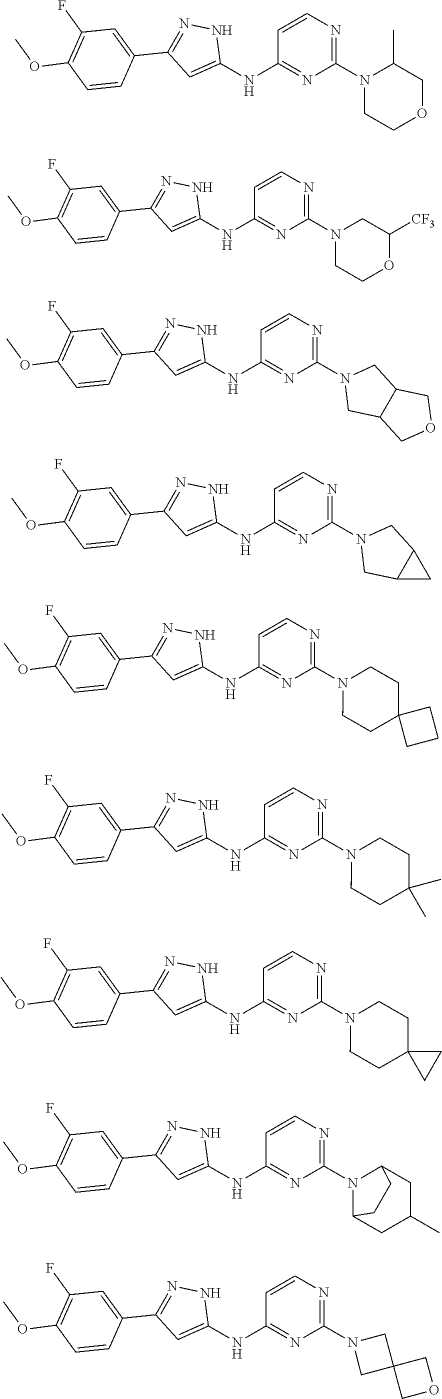 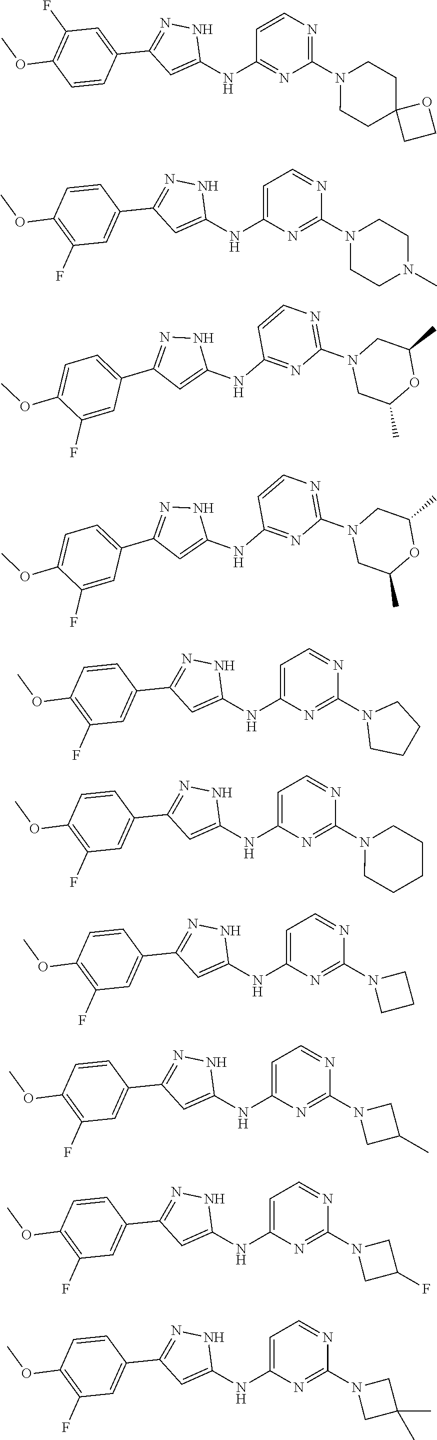 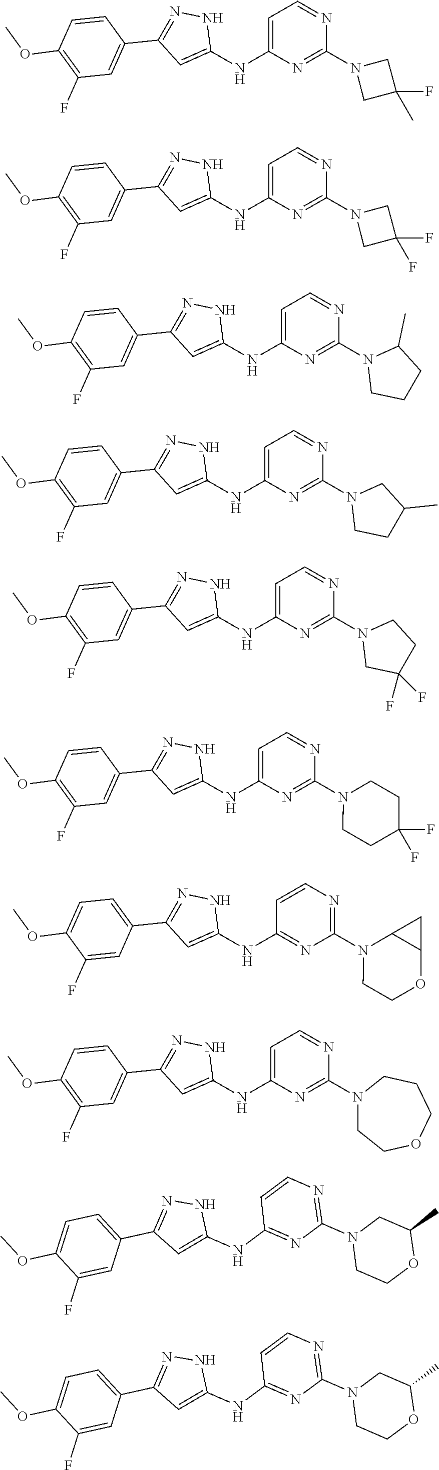 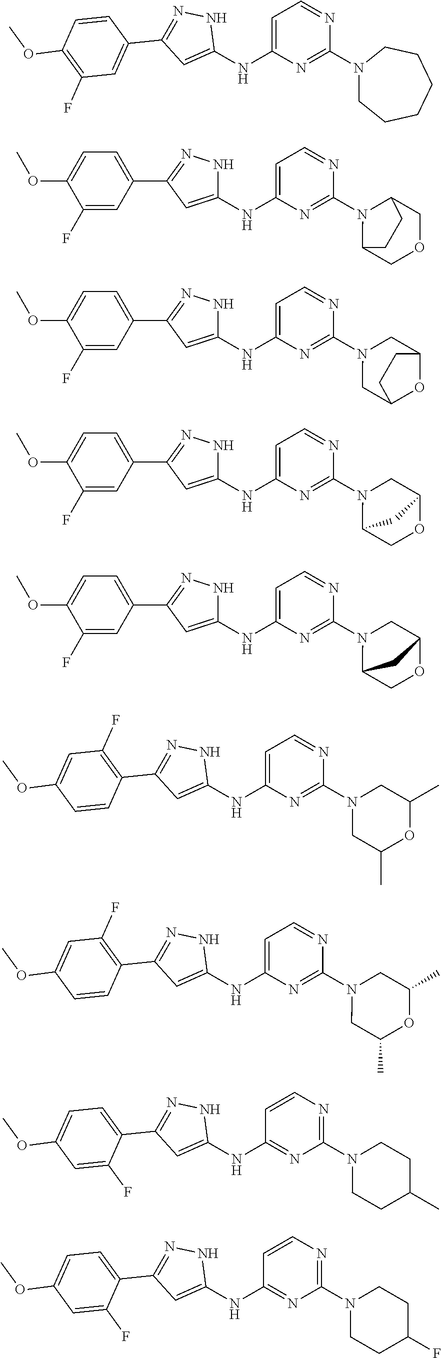 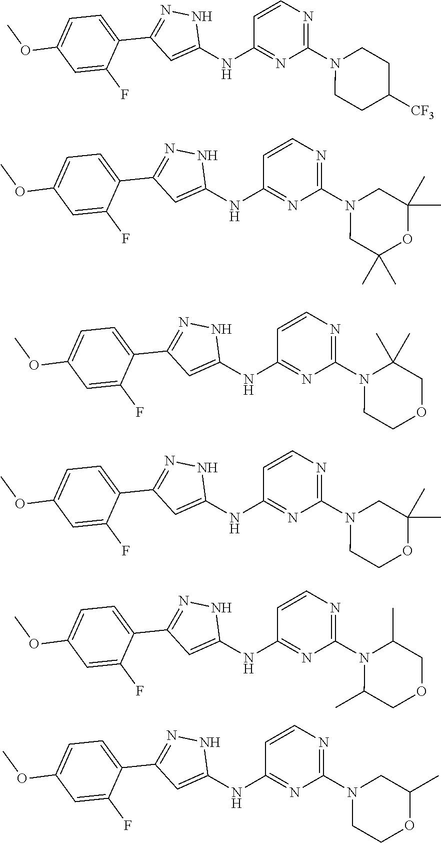 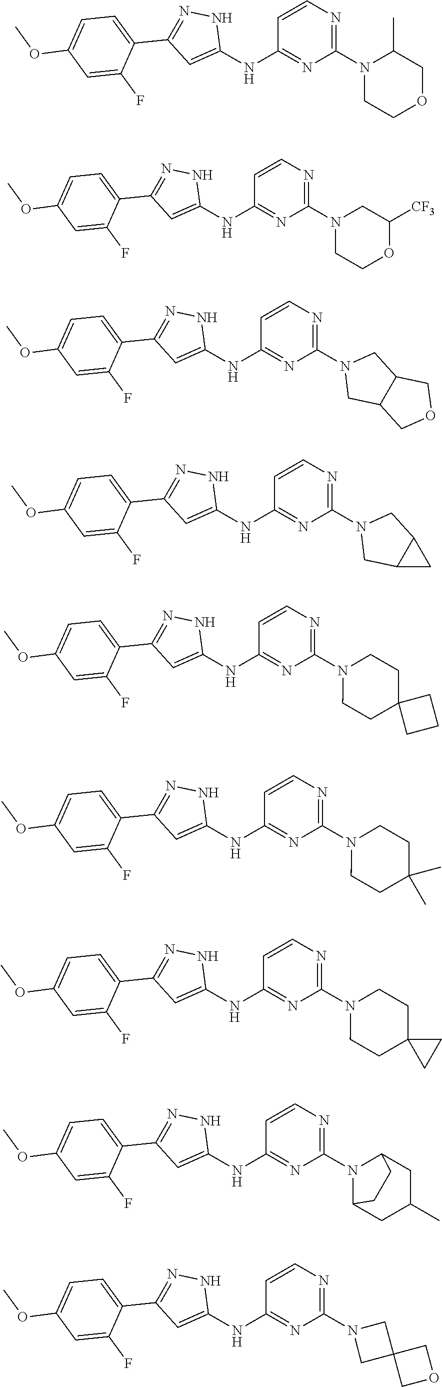 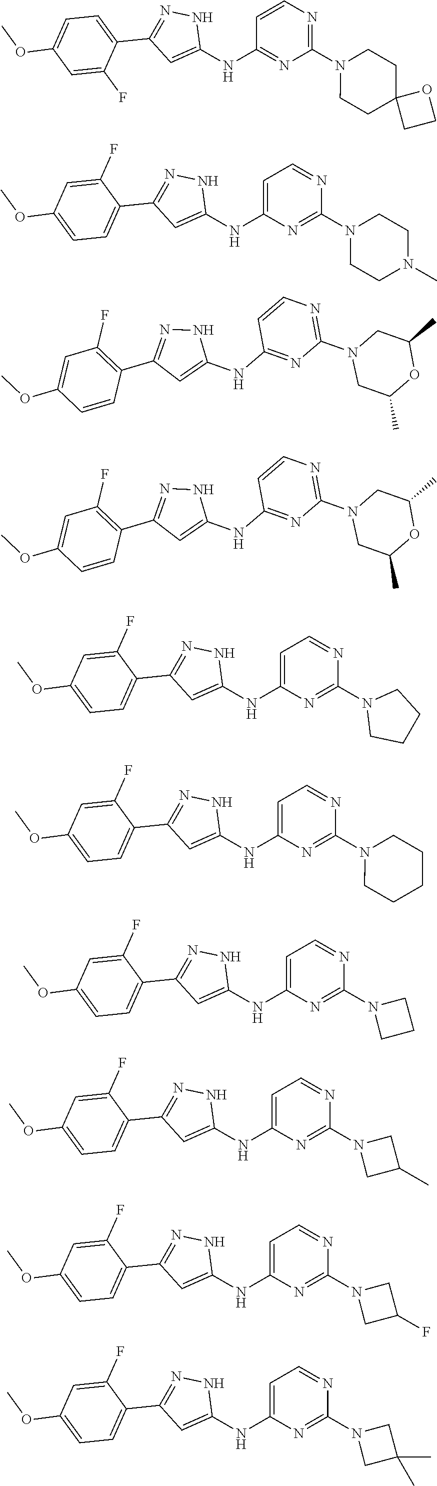 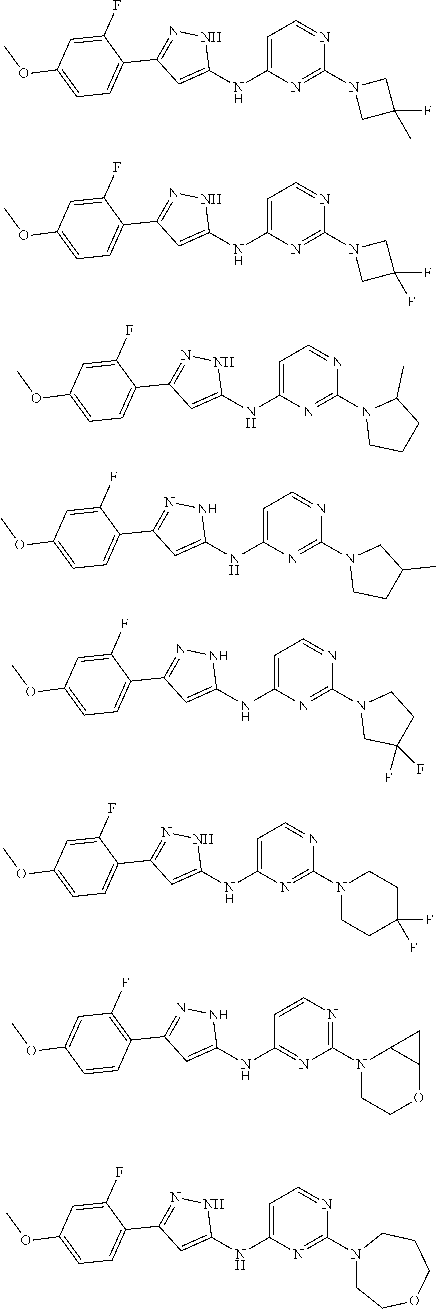 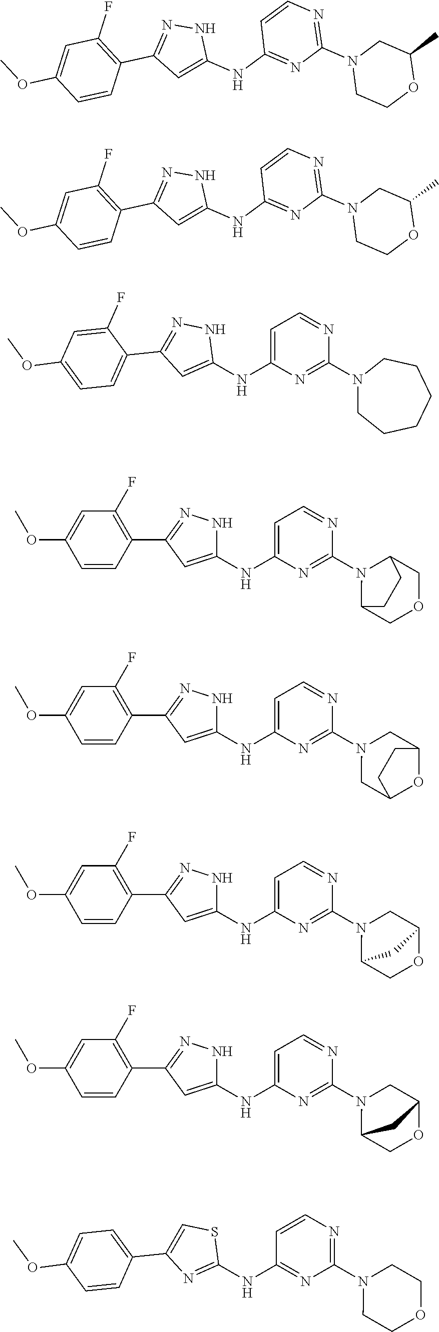 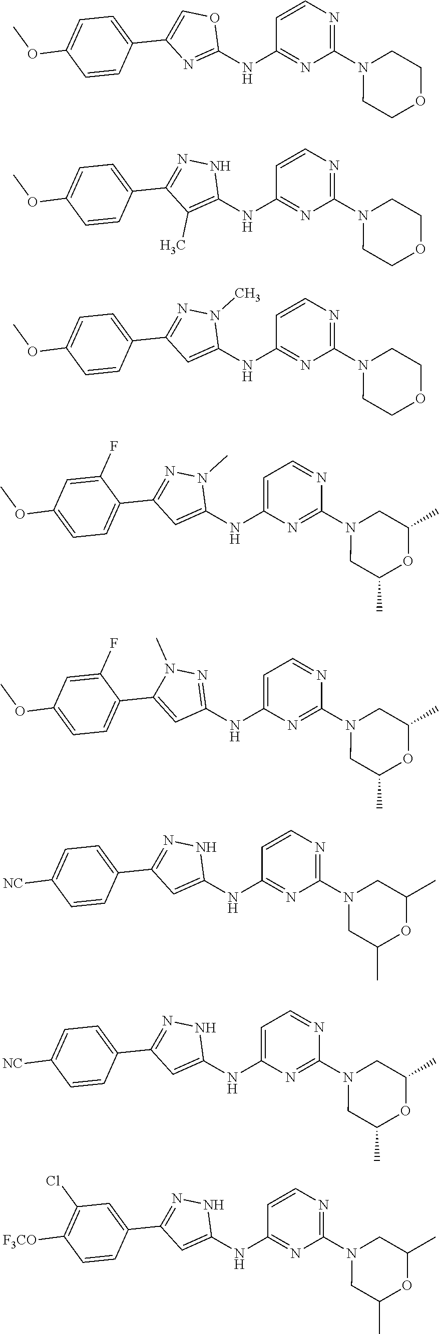 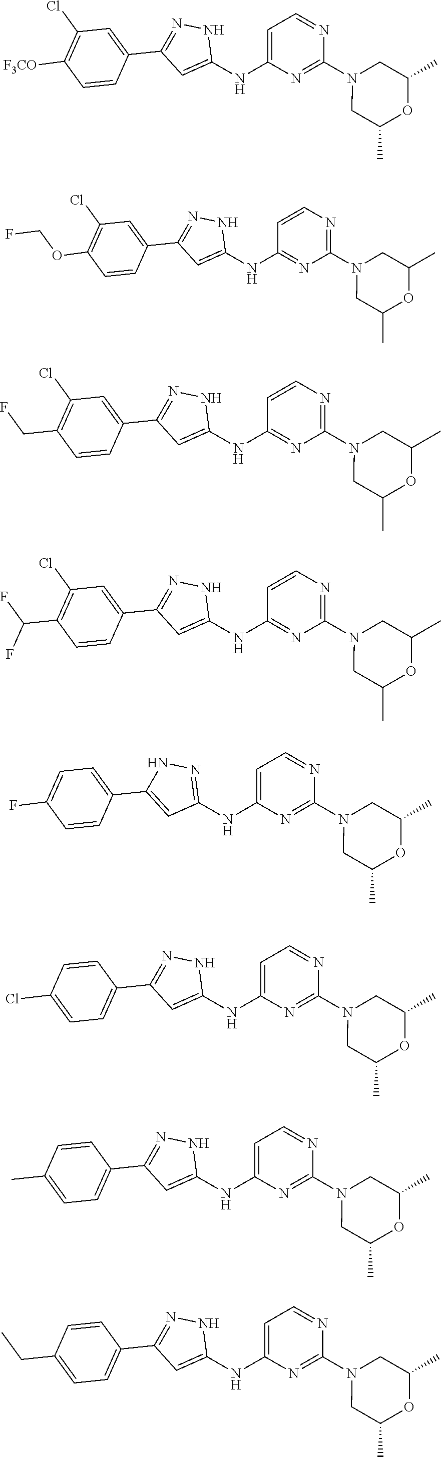 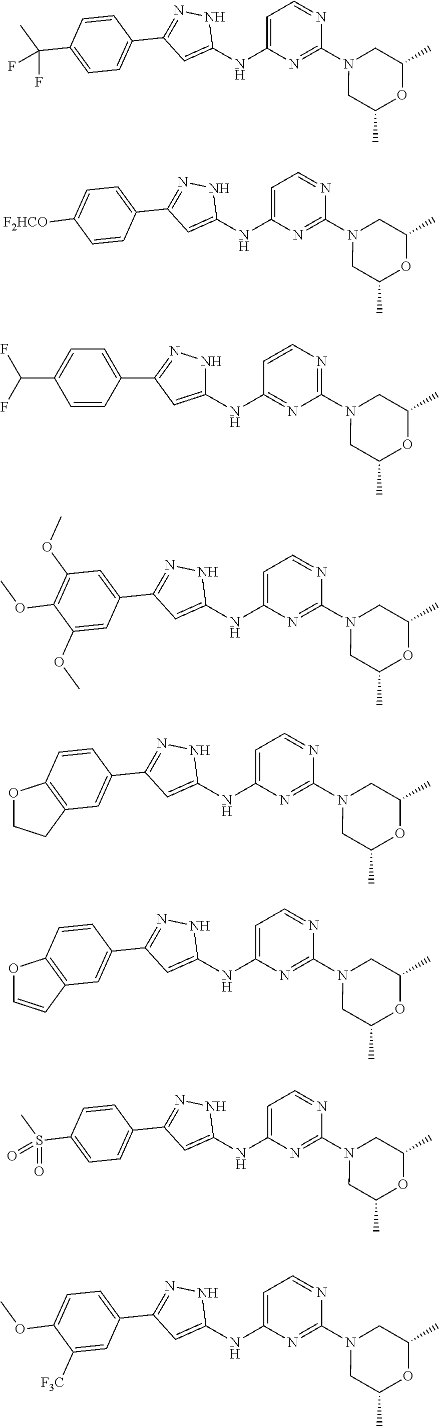 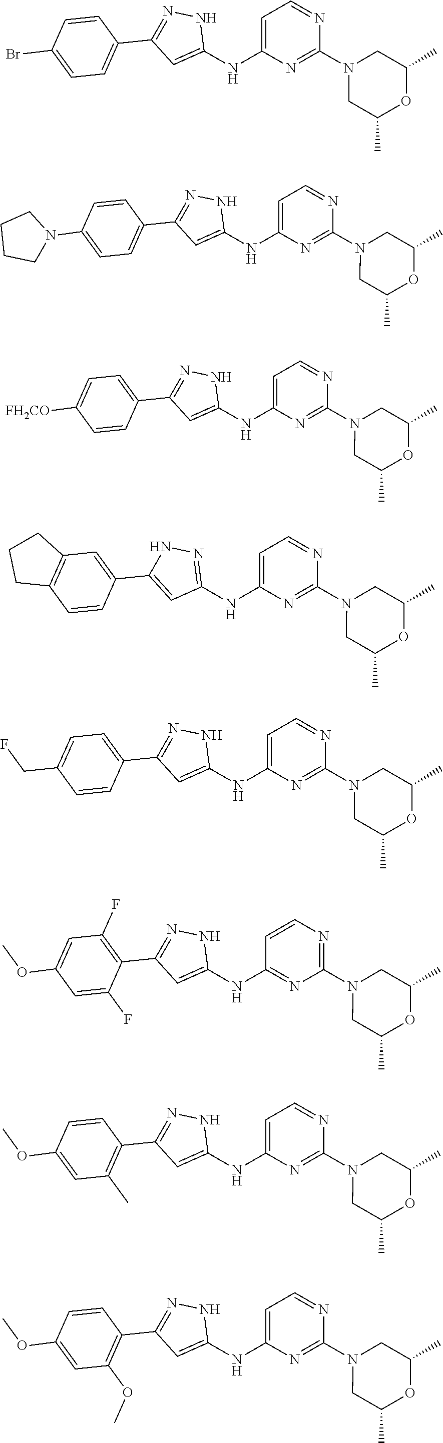 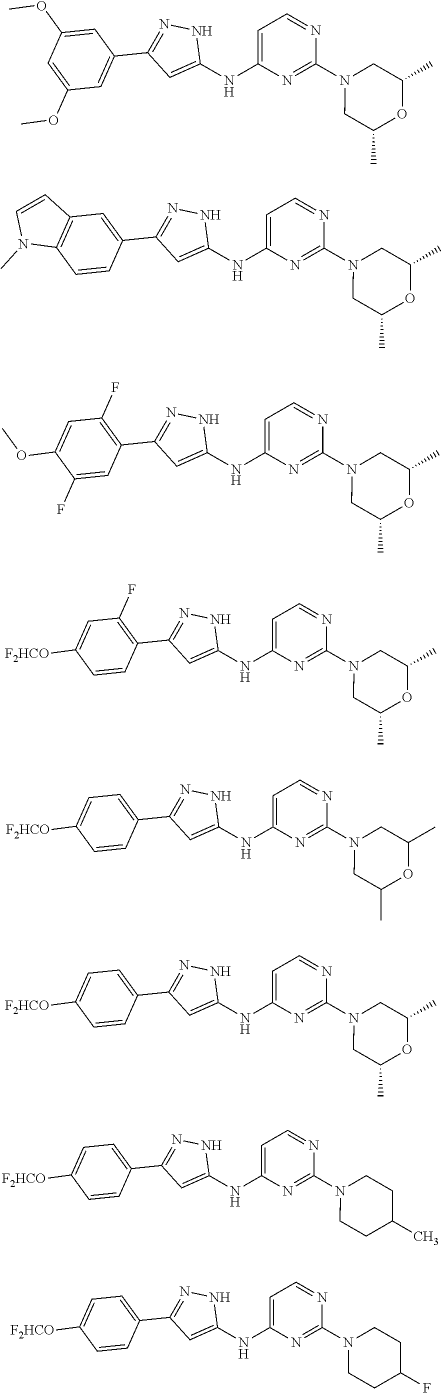 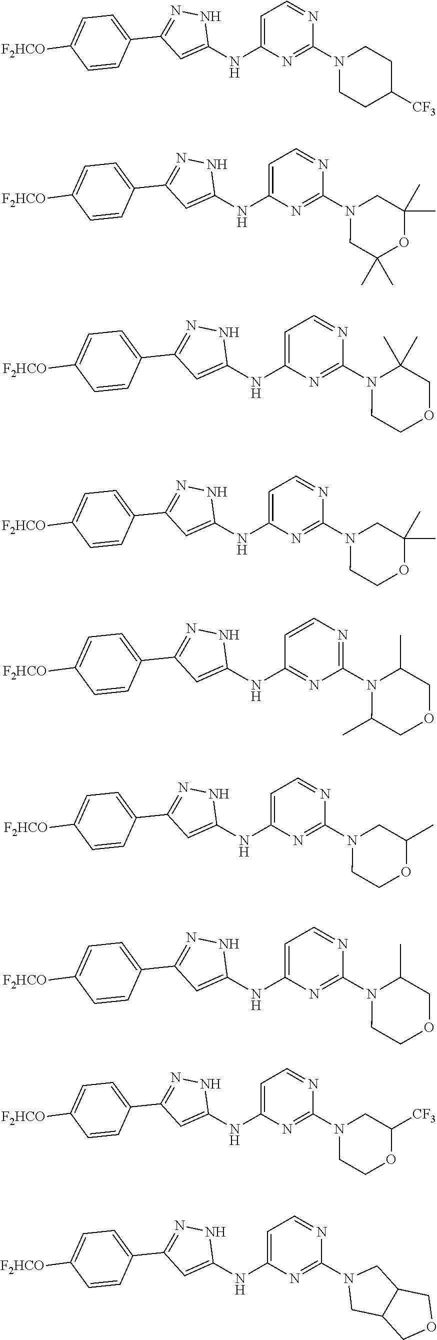 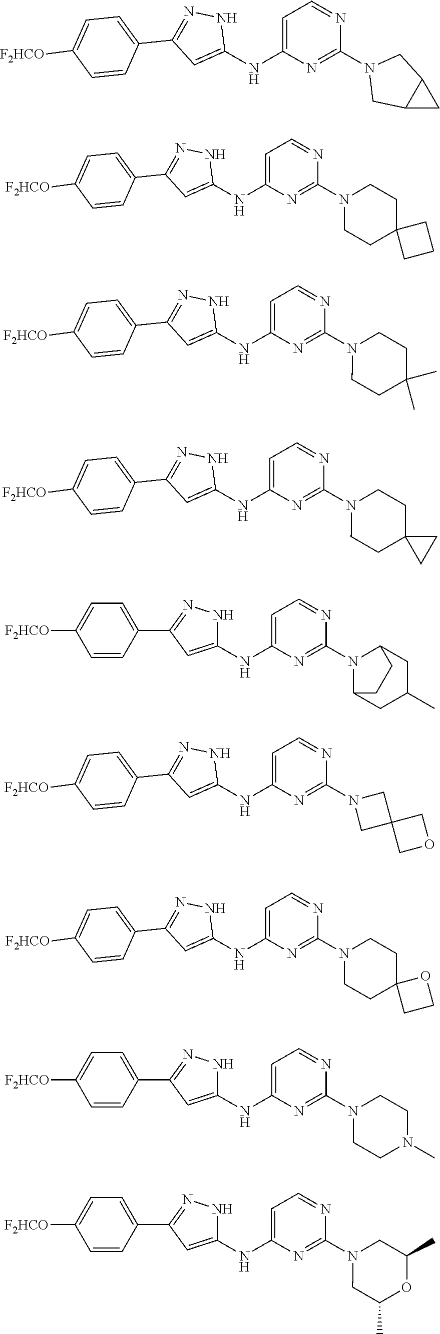 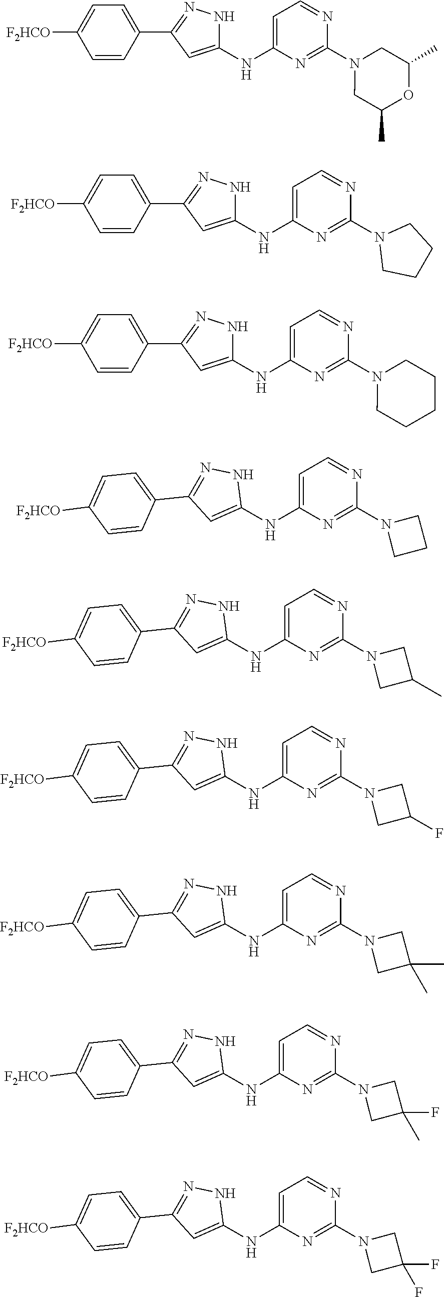 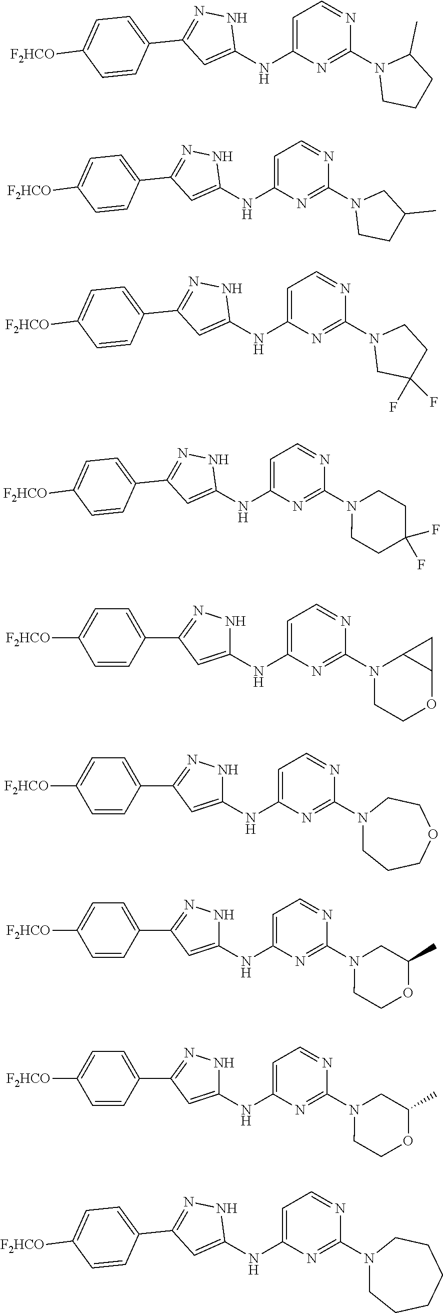 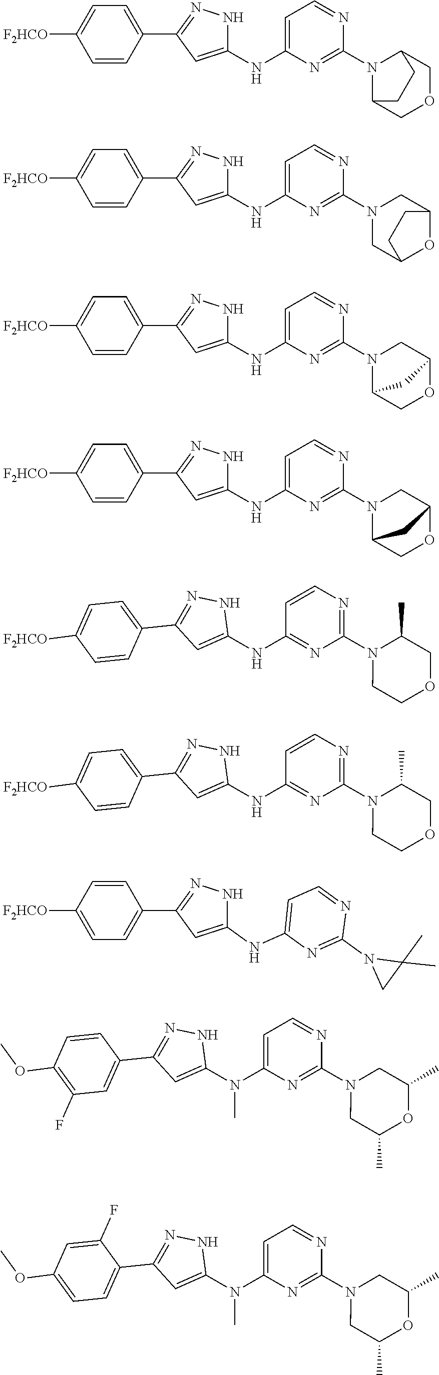 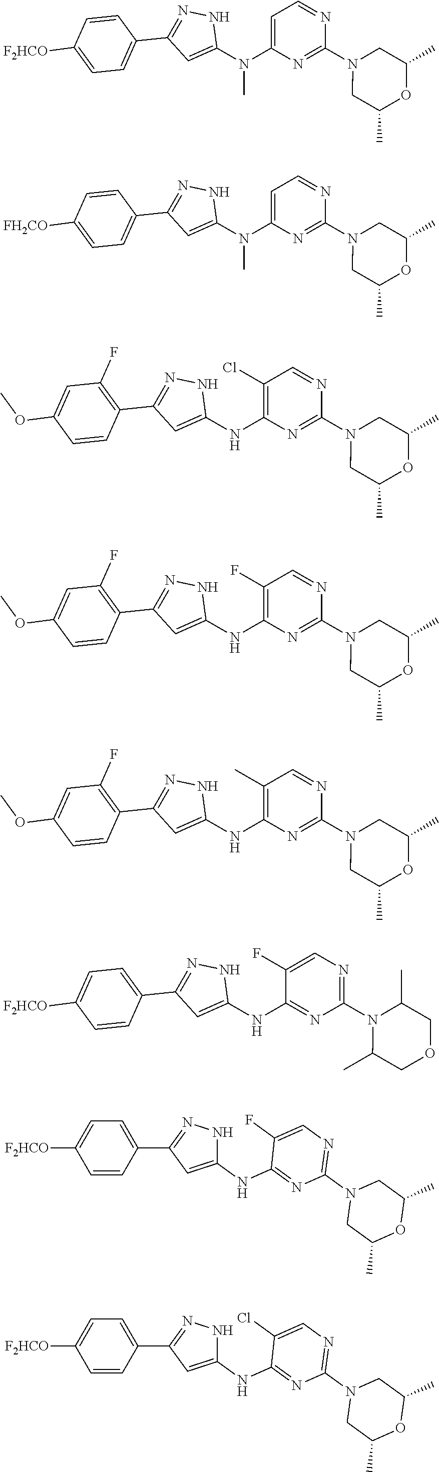 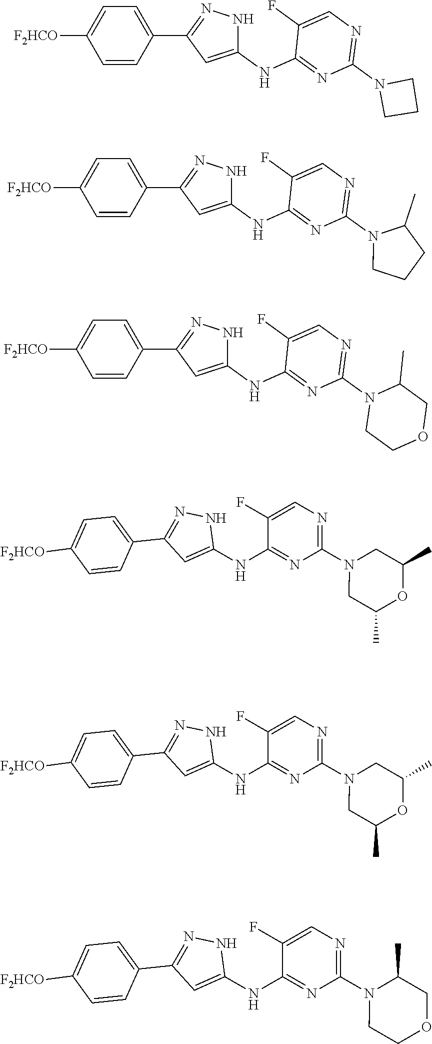 [SMILES: Fc1cnc(N2CCC2)nc1Nc1cc(-c2ccc(OC(F)F)cc2)n[nH]1 ; CC1CCCN1c1ncc(F)c(Nc2cc(-c3ccc(OC(F)F)cc3)n[nH]2)n1 ; CC1COCCN1c1ncc(F)c(Nc2cc(-c3ccc(OC(F)F)cc3)n[nH]2)n1 ; C[C@@H]1CN(c2ncc(F)c(Nc3cc(-c4ccc(OC(F)F)cc4)n[nH]3)n2)C[C@@H](C)O1 ; C[C@H]1CN(c2ncc(F)c(Nc3cc(-c4ccc(OC(F)F)cc4)n[nH]3)n2)C[C@H](C)O1 ; C[C@H]1COCCN1c1ncc(F)c(Nc2cc(-c3ccc(OC(F)F)cc3)n[nH]2)n1] 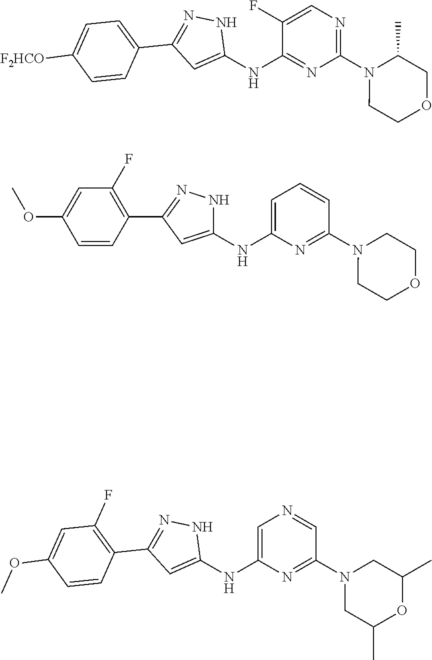 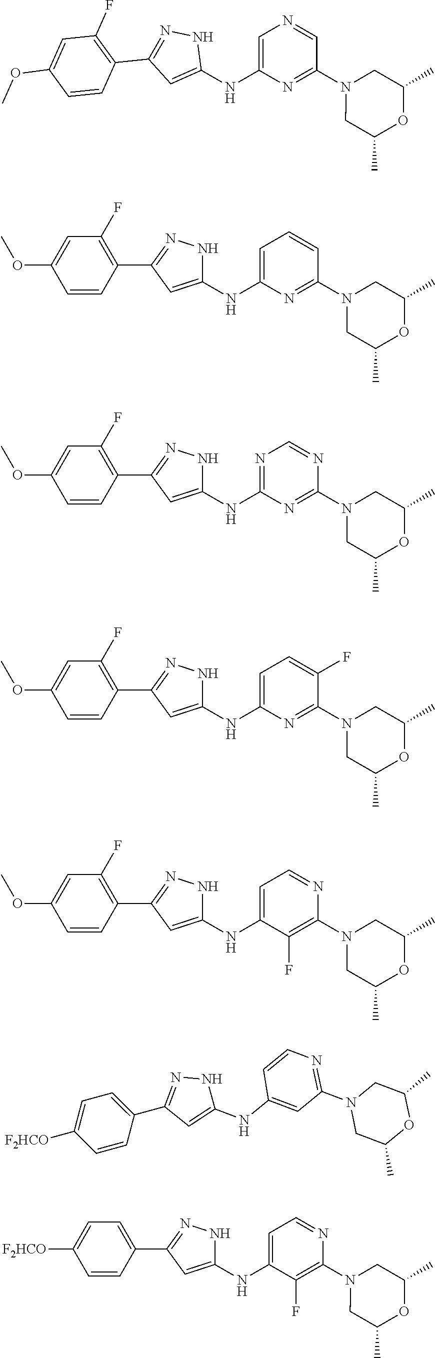 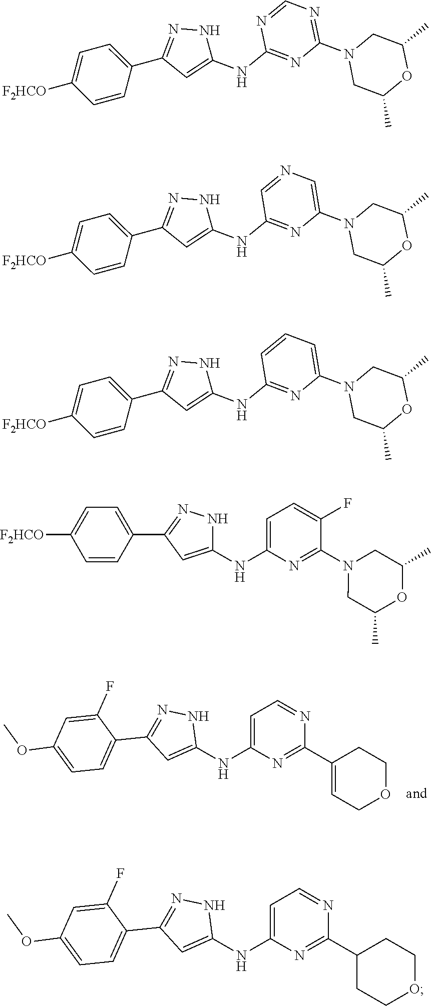
or a pharmaceutically acceptable salt thereof.

86. A pharmaceutical composition comprising a compound of any one of claims 1-85 and a pharmaceutically acceptable excipient.

87. A method of treating a disease or disorder characterized by TACC dysregulation in a subject comprising administering a compound of any one of claims 1-85 or a pharmaceutically acceptable salt thereof to the subject.

88. A method of treating a TACC mediated disease or disorder in a subject comprising administering a compound of any one of claims 1-85 or a pharmaceutically acceptable salt thereof to the subject.

89. The method of claim 87 or 88, wherein the TACC is TACC1.

90. The method of claim 87 or 88, wherein the TACC is TACC2.

91. The method of claim 87 or 88, wherein the TACC is TACC3.

92. The method of any one of claims 87-91, wherein the TACC mediated disease or disorder is cancer.

93. The method of any one of claims 87-91, wherein the disease or disorder is cancer.

94. A method of treating cancer in a subject comprising administering a compound of any one of claims 1-85 or a pharmaceutically acceptable salt thereof to the subject.

95. The method of any one of claims 92-94, wherein the cancer is breast cancer, colon cancer, melanoma cancer, lung cancer, central nervous system cancer, ovarian cancer, leukemia cancer, renal cancer or prostate cancer.
